(19) United States
(12) Patent Application Publication
(10) Pub. No.: US 2025/0340843 A1
(43) Pub. Date: Nov. 6, 2025

(54) METHODS AND COMPOSITIONS FOR SUPPORT OF MYOGENICITY USING CO-CULTURE

(71) Applicant: Upside Foods, Inc., Berkeley, CA (US)

(72) Inventor: Daphne DAMBOURNET, Berkeley, CA (US)

(21) Appl. No.: 18/870,204

(22) PCT Filed: Jun. 5, 2023
(86) PCT No.: PCT/US2023/024485

§ 371 Date: Nov. 27, 2024

Related U.S. Application Data
(60) Provisional application No. 63/349,443, filed on Jun. 6, 2022.

(51) Int. Cl. C12N 5/077; A23L 13/00; C12N 15/85

(52) U.S. Cl. CPC C12N 5/0658; A23L 13/00; C12N 5/0656; C12N 15/85; C12N 2800/106

(57) ABSTRACT

Provided herein are methods of co-culturing a myogenic cell with a support cell to increase myotube formation from the myogenic cells. For example, provided herein is a method comprising co-culturing a myogenic cell with a support cell wherein the support cell comprises a polynucleotide comprising a coding sequence of a gene of interest; and culturing the myogenic cell and the support cell in a cultivation infrastructure under conditions sufficient to induce proliferation of the myoblast cell.

### 1. CROSS-REFERENCE TO RELATED APPLICATIONS

This application claims the benefit of U.S. Provisional Application No. 63/349,443, filed Jun. 6, 2022; which is hereby incorporated in its entirety by reference.

### 2. SEQUENCE LISTING

The instant application contains a Sequence Listing.

### 3. BACKGROUND

The mass production of cells remains limited by several factors, thus limiting final yields. Mass production of cells finds several downstream applications. For example, foods formulated from metazoan cells, cultured in vitro, have prospective advantages over their corporal-derived animal counterparts, including improved nutrition and safety. Production of these products have been projected to require fewer resources, convert biomass at a higher caloric efficiency and result in reduced environmental impacts relative to conventional in vivo methods. Together, metazoan cells, and their extracellular products, constitute a biomass that can potentially be harvested from a cultivation infrastructure for formulation of cell-based food products, such as cultured meat.

However, mass production of cells originating from cultured metazoan cells remains limited by several factors, for example, by the maximum culture density that can be achieved and the requirements for achieving such a density during the cultivation process, thus limiting final yields. Provided herein are compositions and methods that address this and other related needs.

### 4. SUMMARY

This disclosure is based in part on the finding that co-culturing myogenic cells with support cells that include a polynucleotide comprising a coding sequence of a gene of interest (e.g., FAK, FAP, IGF2, SDC4, or SPHK1) improved the myogenic cells ability to form myotubes. This disclosure is also based in part on the finding that co-culturing myogenic cells that include a polynucleotide comprising a coding sequence of a gene of interest (e.g., IGF2) with support cells that comprise a polynucleotide comprising a coding sequence of a gene of interest (e.g., FAK, FAP, IGF2, SDC4, or SPHK1) improved the myogenic cells ability to form myotubes.

Overall, this work demonstrates the ability to enhance myotube formation and subsequent generation of cell based meat products suitable for consumption by co-culturing myogenic cells with support cells engineered to express a gene of interest. These findings are important because manufacturing cells for cell based meat products includes tailoring the methods so that the cells that make up the cell based meat products have appropriate texture profiles. In such cases, these texture profiles can depend, at least in part, on the cell's ability to form myotubes and/or contain muscle proteins such as MyHC. The methods described herein provide a means for increasing the efficiency (e.g., bioconversion efficiency) with which myogenic cells form myotubes expressing MyHC, thereby increasing the efficiency with which cell based meat products that include the desired texture profiles can be produced.

In one aspect, this disclosure features a method for increasing cell density of a culture comprising a myogenic cell line, comprising: (a) co-culturing a myogenic cell with a support cell, wherein the support cell line comprises a polynucleotide comprising a coding sequence of a gene of interest; and (b) culturing the myogenic cell and the support cell in a cultivation infrastructure under conditions sufficient to induce proliferation of the myogenic cell, thereby increasing cell density of the culture.

In some embodiments, the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1 or 1:10 number of myogenic cells to number of support cells.

In some embodiments, the myogenic cell is selected from: a myoblast, a myocyte, a satellite cell, a side population cell, a myogenic pericyte, a mesangioblast, a multinucleated myotube, a skeletal muscle fiber, or a combination thereof.

In some embodiments, the myogenic cells are natively myogenic or are non-natively myogenic.

In some embodiments, the support cell is selected from: a fibroblast, a myofibroblast, a mesenchymal cell, an epithelial cell, and a stromal cell.

In some embodiments, the gene of interest is selected from: FAP, IGF2, SDC4, SPHK1, and FAK, or a combination thereof.

In some embodiments, the myogenic cell comprises a polynucleotide comprising a coding sequence of a gene of interest.

In some embodiments, the gene of interest is IGF2 or genetic variant thereof.

In some embodiments, a myogenic cell co-cultured with the support cell comprising a polynucleotide comprising a coding sequence of a gene of interest comprises a higher proliferation rates as compared to a myogenic cell not cultured with a support cell comprising a polynucleotide comprising a coding sequence of a gene of interest.

In some embodiments, the myogenic cells, the support cells, or both, are immortalized.

In some embodiments, the method further comprises further comprising an immortalizing step, wherein the myogenic cells, the support cells, or both are immortalized.

In some embodiments, the immortalization is selected from a method comprising: transducing with a polynucleotide encoding TERT, transducing with a polynucleotide encoding CDK4/6, transducing with a polynucleotide Cyclin D1, inactivating a gene encoding an inhibitor of cyclin-dependent kinase 4/6 (CDK4/6), inactivating a gene encoding an inhibitor of Cyclin D1, or a combination thereof.

In some embodiments, the co-culturing, culturing steps, or both, comprises contacting the myogenic cell, support cell, or both with a growth medium.

In some embodiments, the growth media comprises one or more of: DMEM/F12, fetal bovine serum, chicken serum, fibroblast growth factor 2, a TGF-beta inhibitor, an activin A inhibitor, and a WNT activator.

In some embodiments, the co-culturing and/or culturing steps comprises contacting the myogenic cell, support cell, or both with a differentiation medium comprising bovine serum, chicken serum, horse serum, or a combination thereof.

In some embodiments, the myogenic cells are from a chicken, a duck, turkey, porcine, or bovine.

In some embodiments, the support cells are from a chicken, a duck, or turkey, porcine, or bovine.

In some embodiments, the method further comprising: inducing myogenic specific differentiation, wherein the differentiated cells form myocytes and multinucleated myotubes, wherein the myocytes and multinucleated myotubes form a skeletal muscle fiber, and isolating the skeletal muscle fiber and producing the cell based meat product suitable for consumption.

In another aspect, this disclosure features a cell-based meat product suitable for consumption produced using any of the methods described herein.

In some embodiments, the cell-based meat product suitable for consumption is a raw, uncooked food product or a cooked food product.

In one aspect, this disclosure features methods for increasing bioconversion efficiency of a myogenic cell, comprising: (a) co-culturing a myogenic cell with a support cell, wherein the support cell line comprises a polynucleotide comprising a coding sequence of a gene of interest; and (b) culturing the myogenic cell and the support cell in a cultivation infrastructure under conditions sufficient to induce formation of the myocytes and multinucleated myotubes from the myogenic cell.

In another aspect, this disclosure features methods for increasing cell density of a culture comprising a myogenic cell line, comprising: (a) co-culturing a myogenic cell with a support cell, wherein the support cell line comprises a polynucleotide comprising a coding sequence of a gene of interest; and (b) culturing the myogenic cell and the support cell in a cultivation infrastructure under conditions sufficient to induce proliferation of the myogenic cell.

In another aspect, this disclosure features methods for increasing myotube formation from a myogenic cell, comprising: (a) co-culturing a myogenic cell with a support cell, wherein the support cell comprises a polynucleotide comprising a coding sequence of a gene of interest; and (b) culturing the myogenic cell and the support cell in a cultivation infrastructure under conditions sufficient to induce myotube formation from the myogenic cell.

In another aspect, this disclosure features methods of producing a cell based meat product suitable for consumption, comprising: (a) co-culturing a myogenic cell and a support cell, wherein the support cell comprises a polynucleotide comprising a coding sequence of a gene of interest; (b) inducing myogenic specific differentiation, wherein the myogenic cells form myocytes and multinucleated myotubes; (c) culturing the myocytes and multinucleated myotubes to generate skeletal muscle fibers; and (d) isolating the skeletal muscle fibers and producing the cell based meat product suitable for consumption.

In some embodiments, the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1 or 1:10 number of myogenic cells to number of support cells.

In some embodiments, the myogenic cell is selected from: a myoblast, a myocyte, a satellite cell, a side population cell, a myogenic pericyte, a mesangioblast, a multinucleated myotube, a skeletal muscle fiber, or a combination thereof.

In some embodiments, the myogenic cells are natively myogenic.

In some embodiments, the myogenic cells are non-natively myogenic.

In some embodiments, the support cell is selected from: a fibroblast, a myofibroblast, a mesenchymal cell, an epithelial cell, and a stromal cell.

In some embodiments, the gene of interest is selected from: FAP, IGF2, SDC4, SPHK1, and FAK, or a combination thereof.

In some embodiments, the gene of interest is FAP. In some embodiments, FAP comprises an amino acid sequence having at least 80% sequence identity to a sequence selected from SEQ ID NOs: 1-11. In some embodiments, FAP comprises an amino acid sequence selected from SEQ ID NO: 1-11.

In some embodiments, the gene of interest is IGF2. In some embodiments, IGF2 comprises an amino acid sequence having at least 80% sequence identity to a sequence selected from SEQ ID NOs: 12-44. In some embodiments, IGF2 comprises an amino acid sequence selected from SEQ ID NO: 12-44.

In some embodiments, the gene of interest is SDC4. In some embodiments, SDC4 comprises an amino acid sequence having at least 80% sequence identity to a sequence selected from SEQ ID NOs: 45-57. In some embodiments, SDC4 comprises an amino acid sequence selected from SEQ ID NO: 45-57.

In some embodiments, the gene of interest is SPHK1. In some embodiments, SPHK1 comprises an amino acid sequence having at least 80% sequence identity to a sequence selected from SEQ ID NOs: 58-83. In some embodiments, SPHK1 comprises an amino acid sequence selected from SEQ ID NO: 58-83.

In some embodiments, the gene of interest is FAK. In some embodiments, FAK comprises amino acid sequence having at least 80% sequence identity to a sequence selected from SEQ ID NOs: 84-96. In some embodiments, FAK comprises an amino acid sequence selected from SEQ ID NO: 84-96.

In some embodiments, the myogenic cell comprises a polynucleotide comprising a coding sequence of a gene of interest.

In some embodiments, the gene of interest is IGF2 or genetic variant thereof.

In some embodiments, the myogenic cell comprises a polynucleotide comprising a coding sequence a myogenic transcription factor. In some embodiments, the myogenic transcription factor is selected from MYOD1, MYOG, MYF5, MYF6, PAX3, PAX7, or genetic variants thereof.

In some embodiments, co-culturing a myogenic cell with a support cell comprising a polynucleotide comprising a coding sequence of a gene of interest results in increased myotube formation, myogenin expression, myosin heavy chain expression, or a combination thereof as compared to a myogenic cell not exposed to any of the methods described herein.

In some embodiments, a myogenic cell co-cultured with a support cell comprising a polynucleotide comprising a coding sequence of a gene of interest comprises higher total protein as compared to a myogenic cell not cultured with the support cell comprising a polynucleotide comprising a coding sequence of a gene of interest.

In some embodiments, a myogenic cell co-cultured with the support cell comprising a polynucleotide comprising a coding sequence of a gene of interest comprises a higher proliferation rates as compared to a myogenic cell not cultured with a support cell comprising a polynucleotide comprising a coding sequence of a gene of interest.

In some embodiments, the myogenic cells, the support cells, or both, are immortalized.

In some embodiments, the method also includes an immortalizing step, wherein the myogenic cells, the support cells, or both are immortalized. In some embodiments, the immortalization is selected from a method comprising: transducing with a polynucleotide encoding TERT, transducing with a polynucleotide encoding CDK4/6, transducing with a polynucleotide Cyclin D1, inactivating a gene encoding an inhibitor of cyclin-dependent kinase 4/6 (CDK4/6), inactivating a gene encoding an inhibitor of Cyclin D1, or a combination thereof.

In some embodiments, the co-culturing, culturing steps, or both, comprises contacting the myogenic cell, support cell, or both with a growth medium.

In some embodiments, the growth media comprises DMEM/F12, fetal bovine serum, chicken serum and fibroblast growth factor 2. In some embodiments, the growth media further comprises a TGF-beta inhibitor, an activin A inhibitor, and a WNT activator.

In some embodiments, the co-culturing and/or culturing steps comprises contacting the myogenic cell, support cell, or both with a differentiation medium. In some embodiments, the differentiation medium bovine serum, chicken serum, horse serum, or a combination thereof.

In some embodiments, the myogenic cells are from a livestock, poultry, game, or aquatic animal species. In some embodiments, the myogenic cells are from a chicken, a duck, or turkey.

In some embodiments, the myogenic cells are from a fish. In some embodiments, the myogenic cells are from a livestock species. In some embodiments, the livestock species is porcine or bovine. In some embodiments, the myogenic cells are from any animal species intended for human or non-human dietary consumption.

In some embodiments, the support cells are from a livestock, poultry, game, or aquatic animal species. In some embodiments, the support cells are from a chicken, a duck, or turkey. In some embodiments, the support cells are from a fish. In some embodiments, the support cells are from a livestock species. In some embodiments, the livestock species is porcine or bovine. In some embodiments, the support cells are from any animal species intended for human or non-human dietary consumption.

In some embodiments, the method also includes inducing myogenic specific differentiation, wherein the differentiated cells form myocytes and multinucleated myotubes.

In some embodiments, the myocytes and multinucleated myotubes form a skeletal muscle fiber.

In some embodiments, the method also includes isolating the skeletal muscle fiber and producing the cell based meat product suitable for consumption.

In another aspect, this disclosure features myogenic cells produced using any of the methods described herein.

In another aspect, this disclosure features cell based meat products suitable for consumption produced using any of the methods described herein.

In another aspect, this disclosure features cell-based meat products suitable for consumption, comprising: (a) a myogenic cell (e.g., any of the myogenic cells described herein); and (b) a support cell (e.g., any of the support cells described herein) comprising a polynucleotide comprising a coding sequence of a gene of interest.

In some embodiments, the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1 or 1:10 number of myogenic cells to number of support cells.

In some embodiments, the myogenic cell is selected from: a myoblast, a myocyte, a satellite cell, a side population cell, a muscle derived stem cell, a myogenic pericyte, a mesangioblast, a multinucleated myotube, a skeletal muscle fiber, or a combination thereof.

In some embodiments, the myogenic cells are natively myogenic.

In some embodiments, the myogenic cells are non-natively myogenic.

In some embodiments, the support cell is selected from: a fibroblast, a myofibroblast, a mesenchymal cell, an epithelial cell, and a stromal cell.

In some embodiments, the gene of interest is selected from: FAP, IGF2, SDC4, SPHK1, and FAK, or a combination thereof.

In some embodiments, the gene of interest is FAP. In some embodiments, FAP comprises an amino acid sequence having at least 80% sequence identity to a sequence selected from SEQ ID NOs: 1-11. In some embodiments, FAP comprises an amino acid sequence selected from SEQ ID NO: 1-11.

In some embodiments, the gene of interest is IGF2. In some embodiments, IGF2 comprises an amino acid sequence having at least 80% sequence identity to a sequence selected from SEQ ID NOs: 12-44. In some embodiments, IGF2 comprises an amino acid sequence selected from SEQ ID NO: 12-44.

In some embodiments, the gene of interest is SDC4. In some embodiments, SDC4 comprises an amino acid sequence having at least 80% sequence identity to a sequence selected from SEQ ID NOs: 45-57. In some embodiments, SDC4 comprises an amino acid sequence selected from SEQ ID NO: 45-57.

In some embodiments, the gene of interest is SPHK1. In some embodiments, SPHK1 comprises an amino acid sequence having at least 80% sequence identity to a sequence selected from SEQ ID NOs: 58-83. In some embodiments, SPHK1 comprises an amino acid sequence selected from SEQ ID NO: 58-83.

In some embodiments, the gene of interest is FAK. In some embodiments, FAK comprises amino acid sequence having at least 80% sequence identity to a sequence selected from SEQ ID NOs: 84-96. In some embodiments, FAK comprises an amino acid sequence selected from SEQ ID NO: 84-96.

In some embodiments, the myogenic cell comprises a polynucleotide comprising a coding sequence of a gene of interest. In some embodiments, the gene of interest is IGF2 or genetic variant thereof.

In some embodiments, the myogenic cell comprises a polynucleotide comprising a coding sequence a myogenic transcription factor. In some embodiments, the myogenic transcription factor is selected from MYOD1, MYOG, MYF5, MYF6, PAX3, PAX7, or genetic variants thereof.

In some embodiments, the myogenic cells are from a livestock, poultry, game, or aquatic animal species. In some embodiments, the myogenic cells are from a chicken, a duck, or turkey. In some embodiments, the myogenic cells are from a fish. In some embodiments, the myogenic cells are from a livestock species. In some embodiments, the livestock species is porcine or bovine. In some embodiments, the myogenic cells are from any animal species intended for human or non-human dietary consumption.

In some embodiments, the support cells are from a livestock, poultry, game, or aquatic animal species. In some embodiments, the support cells are from a chicken, a duck, or turkey. In some embodiments, the support cells are from a fish. In some embodiments, the support cells are from a livestock species. In some embodiments, the livestock species is porcine or bovine. In some embodiments, the support cells are from any animal species intended for human or non-human dietary consumption.

In some embodiments, the cell-based meat product suitable for consumption is a raw, uncooked food product.

In some embodiments, the cell-based meat product suitable for consumption is a cooked food product.

### 5. BRIEF DESCRIPTION OF THE DRAWINGS

These and other features, aspects, and advantages of the present invention will become better understood with regard to the following description, and accompanying drawings, where:

FIG. 1 shows the results as % area of Myosin Heavy Chain (MyHC) for 8D TERT myoblasts co-cultured with genetically engineered chicken fibroblasts (1312 cells) expressing the genes of interest as indicated on the x-axis.

FIG. 2 shows the results as % area of Myosin Heavy Chain (MyHC) for 8D TERT+IGF2 myoblasts co-cultured with genetically engineered chicken fibroblasts (1312 cells) expressing the genes of interest as indicated on the x-axis.

FIGS. 3A-3B shows images of MyHC staining for myoblasts (8D TERT (FIG. 3A)) or 8D TERT+IGF2 (FIG. 3B)) grown without fibroblast co-culture.

FIGS. 4A-4B shows images of MyHC staining for myoblasts (8D TERT (FIG. 4A)) or 8D TERT+IGF2 (FIG. 4B)) co-cultured with fibroblasts not transduced with a polynucleotide comprising a coding sequence of a gene of interest.

FIGS. 5A-5B shows images of MyHC staining for myoblasts (8D TERT (FIG. 5A)) or 8D TERT+IGF2 (FIG. 5B)) co-cultured with fibroblasts transduced with a polynucleotide comprising a coding sequence for FAK.

FIGS. 6A-6B shows images of MyHC staining for myoblasts (8D TERT (FIG. 6A)) or 8D TERT+IGF2 (FIG. 6B)) co-cultured with fibroblasts transduced with a polynucleotide comprising a coding sequence of FAP.

FIGS. 7A-7B shows images of MyHC staining for myoblasts (8D TERT (FIG. 7A)) or 8D TERT+IGF2 (FIG. 7B)) co-cultured with fibroblasts transduced with a polynucleotide comprising a coding sequence of IGF2.

FIGS. 8A-8B shows images of MyHC staining for myoblasts (8D TERT (FIG. 8A)) or 8D TERT+IGF2 (FIG. 8B)) co-cultured with fibroblast transduced with a polynucleotide sequence comprising a coding sequence of SDC4.

FIGS. 9A-9B shows images of MyHC staining for myoblasts (8D TERT (FIG. 8A)) or 8D TERT+IGF2 (FIG. 9B)) co-cultured with fibroblast transduced with a polynucleotide sequence comprising a coding sequence of SPHK1.

FIGS. 10A-10D show an overview diagram of growing and processing different types of cells in accordance with one or more embodiments of the present disclosure. FIG. 10A shows tissue collection, processing, culturing, and cryopreserving. FIG. 10B shows immortalizing, culturing until confluency, suspension culturing, cryopreserving, and expanding in larger culture vessels. FIG. 10C shows a bioreactor system. FIG. 10D shows a pressure apparatus that compresses cell masses.

### 6. DETAILED DESCRIPTION

Provided herein are methods for co-culturing myogenic cells with support cells engineered to overexpress a gene of interest. In particular, this disclosure is based in part on the finding that co-culturing a myogenic cell with a support cell (e.g., a fibroblast cell) engineered to express a gene of interest enhances the myogenic cell's ability to form myotubes as compared to myogenic cells not co-cultured with support cells or co-cultured with support cells that are not engineered to express a gene of interest. For example, this disclosure features a method for improving myotube formation from a myogenic cell by co-culturing the myogenic cell in a cultivation infrastructure with support cells (e.g., fibroblasts) engineered to express a gene of interest (e.g., FAK, FAP, IGF2, SDC4, and SPHK1, or a combination thereof).

### 6.1 Definitions

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Throughout this disclosure, the term “a” or “an” entity refers to one or more of that entity. As such, the terms “a” (or “an”), “one or more,” and “at least one” can be used interchangeably herein. Furthermore, “and/or” as used in a phase such as “A and/or B” herein is intended to include “A and B,” “A” (alone), and “B” (alone). Likewise, the term “and/or” as used in a phrase such as “A, B, and/or C” is intended to encompass each of the following aspects: A, B, and C; A, B, or C; A or C; A or B: B or C; A and C; A and B; B and C: A (alone); B (alone); and C (alone).

As used herein, the terms “bioconversion efficiency” or “BCE” refer to the efficiency by which a myogenic cell differentiates into a multinucleated myotube and/or skeletal muscle tissue.

As used herein, the terms “cell” and “cell line” are sometimes used interchangeably. As used herein, the term “cell” can refer to one or more cells originating from a cell line. As used herein, the term “cell line” can refer to a population of cells.

As used herein, the terms “cell surface” or “surface of the cell” when referring to a receptor refers to the presence of the receptor on the surface of the cell.

As used herein, the terms “comprising” and “including” or grammatical variants thereof are to be taken as specifying the stated features, integers, steps, or components but do not preclude the addition of one or more additional features, integers, steps, components, or groups thereof. This term encompasses the terms “consisting of” and “consisting essentially of”.

As used herein, the terms “co-culture” or “co-culturing” refer to act or process of culturing two types of cells or tissue in the same medium.

As used herein, the term “cultivation infrastructure” refers to the environment in which liver-derived cells, dedifferentiated cell, or myogenic cells (e.g., non-naturally occurring myogenic cells) are cultured (i.e., the environment in which the myogenic cell is cultivated).

As used herein, the term “differentiation capacity” refers to a cells ability to differentiate to a particular cell lineage, stem cell, progenitor cell, or terminally differentiated cell.

As used herein, the term “engineered” refers to a cell containing an exogenous polynucleotide that includes a coding sequence of a gene of interest. The term “engineered to express” refers to introduction of an exogenous polynucleotide that includes a coding sequence of a gene of interest into a cell, whereby the cell expresses the gene of interest or is capable of expressing the gene of interest.

As used herein, the term “exogenous” when referring to a growth factors refers to a growth added to the culture medium.

As used herein, the term “fragment” or “portion” when referring a protein or a polynucleotide refers to a protein that comprises a domain, portion, or fragment of a parent or reference protein or polypeptide. Ther term “portion” can be used interchangeably with the term “functional portion.” The term “fragment” can be used interchangeably with the term “functional fragment.” The terms “functional portion” or “functional fragment” retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent protein or polypeptide, or provides a biological benefit. A “functional portion” or “functional fragment” of a protein or polypeptide has “similar binding” or “similar activity” when the functional portion or fragment displays no more than a 50% reduction in performance in a selected assay as compared to the parent or reference protein or polypeptide (preferably no more than 20% or 10%, or no more than a log difference as compared to the parent or reference with regard to affinity).

As used herein, the term “immortalized cell” refers to cells that are passaged or modified to proliferate indefinitely and evade normal cellular senescence.

As used herein, the term “myoblast” refers to mononucleated muscle cells. They are embryonic precursors of myocytes, also called muscle cells. Although myoblasts may be classified as skeletal muscle myoblasts, smooth muscle myoblasts, and cardiac muscle myoblasts depending on the type of muscle cell that they will differentiate into, in this specification the term myoblasts refer to skeletal muscle myoblasts.

As used herein, the term “myotube” refers to elongated structures, the result of differentiated myoblast. Upon differentiation, myoblasts fuse into one or more nucleated myotubes and express skeletal muscle markers.

As used herein, the term “myogenicity” refers to a cells ability to become a myogenic cell, maintain myogenic characteristics (i.e., characteristics associated with a myogenic cell), or acquire enhanced myogenic properties (i.e., properties associated with a myogenic cell). “Myogenicity” can be used interchangeably with “myogenesis.”

As used herein, the term “population doubling level (PDL)” refers to the total number of times the cells in the population have doubled since their primary isolation in vitro. Mathematically this is described as n=3.32 (log UCY−log 1)+X, where n=the final PDL number at end of a given subculture, UCY=the cell yield at that point, 1=the cell number used as inoculum to begin that subculture, and X=the doubling level of the inoculum used to initiate the subculture being quantitated.

As used herein the term “passaged cell” refers to the number of times the cells in the culture have been subcultured. This may occur without consideration of the inoculation densities or recoveries involved.

As used herein, the term “substantially free of” or “substantially free from” means the amount (e.g., absolute number within a population or concentration/percentage within a population) of a cell or cell type is below a value where the cell or cell type, or any cell derived therefrom, could contribute to the population. For example, a population substantially free of a cell means that upon differentiation of the population the cell does not contribute progeny to the differentiated population.

As used herein, the term “support cell” refers to a cell that enhances, support and/or maintain proliferation rates of a myogenic cell; promotes cell survival of a myogenic cell; promotes increased myogenic differentiation of a myogenic cell; or increases a myogenic cell's ability to form myotubes, skeletal muscle fiber, or a combination thereof.

As used herein, the terms “transformed,” “transduced,” and “transfected” are used interchangeably unless otherwise noted. Each term refers to introduction of a nucleic acid sequence or polypeptide into a cell (e.g., an immortalized cell).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### 6.2 Co-Culturing Myogenic Cells with Support Cells

Provided herein are methods of co-culturing a myogenic with a support cell, where the support cell comprises a polynucleotide comprising a coding sequence of a gene of interest. In some embodiments, the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1, 1:10, 1:11, 11:1, 1:12, 12:1, 1:13, 13:1, 1:14, 14:1, 1:15, 15:1, 1:16, 16:1, 1:17, 17:1, 1:18, 18:1, 1:19, 19:1, 1:20, or 20:1 of the number of myogenic cells to the number of support cells. In some embodiments, the myogenic cell and the support cell are co-cultured at a ratio of 1:1 of the number of myogenic cells to the number of support cells.

### Myogenic Cells

In some embodiments, the myogenic cells are myoblasts, myocytes, satellite cells, side population cells, muscle derived stem cells, mesenchymal stem cells, myogenic pericytes, or mesangioblasts.

In some embodiments, the myogenic cells are modified to overexpress the coding sequence of an IGF2 protein. In some embodiments, the myogenic cells are genetically modified and carry stable integrations of one or more copies of an IGF2 coding sequence. In some embodiments, the support cells overexpress the coding sequence of an IGF2 protein at levels sufficient to increase production and/or secretion of IGF2 into the cell culture media. In some embodiments, the IGF2 protein can be any of the IGF2 proteins described in Section 4.3.2.

In some embodiments, a myogenic cell is engineered to express an IGF2 protein comprising an amino acid sequence having at least 80% ((e.g., at least 85%, 90%, 95%, 96%, 97%, 98%, or 99%) sequence identity to a sequence selected from SEQ ID NOs: 12-44. In some embodiments, a myogenic cells is engineered to express an IGF2 protein sequence comprising an amino acid sequence selected from SEQ ID NOs: 12-44.

In some embodiments, the myogenic cells are immortalized (e.g., immortalized according to the methods as described herein).

In some embodiments, the myogenic cells are from a livestock, poultry, game, or aquatic animal species. In some embodiments, the myogenic cells are from a chicken, a duck, or turkey. In some embodiments, the myogenic cells are from a fish. In some embodiments, the myogenic cells are from a livestock species. In some embodiments, the livestock species is porcine or bovine. In some embodiments, the myogenic cells are from any animal species intended for human or non-human dietary consumption.

In some embodiments, the myogenic cell is derived from a species selected from including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, the myogenic cell is isolated from Gallus gallus (chicken). In some embodiments, the myogenic cell is isolated from chicken muscle.

In some embodiments, the myogenic cells are genetically modified to inhibit a pathway, e.g. the HIPPO signaling pathway. Exemplary methods to inhibit the HIPPO signaling pathway as described in a PCT Application No. PCT/US2018/031276, which is herein incorporated by reference in its entirety.

In some embodiments, the myogenic cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in a PCT publication, WO 2017/124100, which is herein incorporated by reference in its entirety.

In some embodiments, the myogenic cells are modified to express glutamine synthetase (GS), insulin-like growth factor (IGF), and/or albumin. Exemplary methods of modifying myogenic cells to express GS, IGF, and/or albumin are described in a PCT Application No. PCT/US2018/042187, which is herein incorporated by reference in its entirety.

In some embodiments the myogenic cells are genetically edited, modified, or adapted to grow without the need of specific ingredients including specific amino acids, carbohydrates, vitamins, inorganic salts, trace metals, TCA cycle intermediates, lipids, fatty acids, supplementary compounds, growth factors, adhesion proteins and recombinant proteins.

In some embodiments, the myogenic cells may comprise any combinations of the modifications described herein.

In some embodiments, the cells are non-myogenic, and such non-myogenic cells can be programmed to be myogenic, for example the cells may comprise fibroblasts modified to express one or more myogenic transcription factors. In some embodiments, the myogenic transcription factors include MYOD1, MYOG, MYF5, MYF6, PAX3, PAX7, paralogs, orthologs, and genetic variants thereof. In some embodiments, the cells are modified to express one or more myogenic transcription factors as described in a PCT publication, WO/2015/066377, which is herein incorporated by reference in its entirety.

Also provided herein are cell banks comprising any of the myogenic cells generated according to the methods described herein.

### Support Cells

In some embodiments, a support cell is selected from a fibroblast, a myofibroblast, a mesenchymal cell, an epithelial cell, and a stromal cell.

In some embodiments, the support cells impact the myogenic cells through paracrine interactions. In some embodiments, the paracrine interactions are the result of the support cell being engineered to express a gene of interest.

In some embodiments, the support cells impact the myogenic cells through juxtacrine signaling. In some embodiments, the juxtacrine signaling is a result of the support cell being engineered to express a gene of interest.

In some embodiments, the support cells are immortalized (e.g., immortalized according to the methods described herein).

In some embodiments, the support cells are from a livestock, poultry, game, or aquatic animal species. In some embodiments, the support cells are from a chicken, a duck, or turkey. In some embodiments, the support cells are from a fish. In some embodiments, the support cells are from a livestock species. In some embodiments, the livestock species is porcine or bovine. In some embodiments, the support cells are from any animal species intended for human or non-human dietary consumption.

In some embodiments, the support cell is derived from a species selected from including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, the support cell is isolated from Gallus gallus (chicken). In some embodiments, the support cell is isolated from chicken skin (e.g., dermal fibroblast).

In some embodiments the support cells are genetically edited, modified, or adapted to grow without the need of specific ingredients including specific amino acids, carbohydrates, vitamins, inorganic salts, trace metals, TCA cycle intermediates, lipids, fatty acids, supplementary compounds, growth factors, adhesion proteins and recombinant proteins.

In some embodiments, the support cells may comprise any combinations of the modifications described herein.

In some embodiments, the support cells are modified to express telomerase reverse transcriptase (TERT) and/or inhibit cyclin-dependent kinase inhibitors (CKI). In some embodiments, the support cells are modified to express TERT and/or inhibit cyclin-dependent kinase inhibitors as described in a PCT publication, WO 2017/124100, which is herein incorporated by reference in its entirety.

In some embodiments, the support cells are modified to express glutamine synthetase (GS), insulin-like growth factor (IGF), and/or albumin. Exemplary methods of modifying support cells to express GS, IGF, and/or albumin are described in a PCT Application No. PCT/US2018/042187, which is herein incorporated by reference in its entirety.

Provided herein are cells derived from the cell line(s). Non-limiting examples of cells derived from the immortalized cells (e.g., using the methods described herein) include myoblasts, myotubes, multinucleated myotubes, satellite cells, skeletal muscle fibers, or any combination thereof.

Also provided herein are cell banks comprising any of the support cells generated according to the methods described herein.

Also provided herein are cell banks comprising a myogenic cell (e.g., any of the myogenic cells described herein) and a support cell (e.g., any of the support cell described herein).

### 6.3 Genes of Interest

Provided herein are methods for co-culturing a myogenic cell with a support cell, where the support cell comprises a polynucleotide comprising a coding sequence of a gene of interest. In some embodiments, introducing the polynucleotide comprising the coding sequence of the gene of interest into the support cells results in the cells having increased ability to support the growth of the myogenic cells when in co-culture. In such cases, the cells are engineered to overexpress the coding sequence of the gene of interest. In some embodiments, the support cells are genetically engineered to have stable integration of the one or more copies of a coding sequence for a gene of interest. In some embodiments, the support cells overexpress the coding sequence of the gene of interest at levels sufficient to increase the support cell's ability to enhance, support and/or maintain proliferation rates of the myogenic cells. In some embodiments, the support cells overexpress the coding sequence of the gene of interest at levels sufficient to increase the support cell's ability to promote cell survival of the myogenic cells. In some embodiments, the support cells overexpressing the coding sequence of the gene of interest at levels sufficient to increase the support cell's ability to formation of skeletal muscle fibers from the myogenic cells.

In some embodiments, the gene of interest is selected from a gene involved in Integrin signaling. Non-limiting examples of genes involved in integrin signaling include, without limitation: AKT1, AKT2, AKT3, ARAF, ARHGEF7, BCAR1, BRAF, CAPN10, CAPN11, CAPN1, CAPN2, CAPN3, CAPN5, CAPN6, CAPN7, CAPN9, CAPNS1, CAV1, CAV2, CAV3, CDC42, CRK, CSK, DOCK1, FYN, GIT2, GRB2, HRAS, ILK, ITGA10, ITGA11, ITGA1, ITGA2, ITGA2B, ITGA3, ITGA4, ITGA5, ITGA6, ITGA7, ITGA8, ITGA9, ITGAD, ITGAE, ITGAL, ITGAM, ITGAV, ITGAX, ITGB1, ITGB2, ITGB3, ITGB4, ITGB5, ITGB6, ITGB7, ITGB8, MAP2K1, MAP2K2, MAP2K6, MAPK10, MAPK12, MAPK1, MAPK4, MAPK7, MYLK2, MYO, MYO-P, PAK1, PAK2, PAK3, PAK4, PAK6, PDPK1, PI5K, PIK3R2, PTK2, PXN, RAC1, RAC2, RAC3, RAF1, RAP1A, RAP1B, RAPGEF1, RCJMB04_36h7, RCJMB04_5i17, RCJMB04_8f10, RHO, ROCK1, ROCK2, SEPP1, SHC1, SHC3, SORBS1, SOS1, SRC, TLN1, TNS1, VASP, VAV2, VAV3, VCL, and ZYX

In some embodiments, the gene of interest is selected from a gene involved in mTor signaling. Non-limiting examples of genes involved in mTor signaling, include, without limitation: ADP, AKT1S1, AKT1S1, ATP, Active mTORC1 complex, EEF2K, EIF4B, EIF4E, EIF4EBP1, EIF4E, EIF4G1, Energy dependent, regulation of mTOR, by LKB1-AMPK, GDP, GTP, L-Arg, LAMTOR1, LAMTOR1, LAMTOR2, LAMTOR2, LAMTOR3, LAMTOR3, LAMTOR4, LAMTOR4, LAMTOR5, LAMTOR5, MLST8, MTOR, PI(3,4,5)P3, RHEB, RHEB:GDP, RHEB:GTP, RPS6KB1, RPS6, RPTOR, RRAGA, RRAGA, RRAGB:GTP, RRAGB, RRAGC, RRAGC, RRAGD:GDP, RRAGD, Ragulator, SLC38A9, SLC38A9, TSC1, TSC1:Inhibited TSC2-1-P, TSC1:TSC2, TSC2, TSC2, YWHAB, and YWHAB.

In some embodiments, the gene of interest is selected from FAK, FAP, IGF2, SDC4, and SPHK1, or a combination thereof. In some embodiments, the methods provided herein include a support cell comprising a polynucleotide that comprises a coding sequence of FAK, FAP, IGF2, SDC4, and SPHK1, or a combination thereof.

In some embodiments, the support cell comprises polynucleotides comprising two, three, four, five, six, seven, eight, nine, or ten or more genes of interest.

In some embodiments, the myogenic cells comprise a polynucleotide comprising a coding sequence of a gene of interest. In one embodiment, the method includes co-culturing a myogenic cell comprising a polynucleotide comprising a coding sequence of a gene of interest and a support cell comprising a polynucleotide comprising a coding sequence of a gene of interest. For example, the method includes co-culturing a myogenic cell comprising a polynucleotide comprising a coding sequence of a IGF2 and a support cell comprising a polynucleotide comprising a coding sequence of one or more of FAK, FAP, IGF2, SDC4, and SPHK1, or a combination thereof.

Provided herein are methods where the support cell comprises a two or more populations of support cells were each population is engineered to express a gene of interest. For example, the support cells include a first population engineered to express a first gene of interest (e.g., any one or more of FAK, FAP, IGF2, SDC4, and SPHK1, or a combination thereof) and a second population engineered to express a second gene of interest (e.g., any one or more of FAK, FAP, IGF2, SDC4, and SPHK1, or a combination thereof).

### Fibroblast Activation Protein

In some embodiments, the methods provided herein include a support cell comprising a polynucleotide comprising a coding sequence of fibroblast activation protein (FAP) or a fragment thereof. As used herein, “FAP” refers to the fibroblast activation protein alpha (Fap) gene or FAP protein that is a member of the serine protease family. Without wishing to be bound by theory, FAP is involved in the control of fibroblast growth or epithelial-mesenchymal interactions during development and tissue repair, participates in extracellular matrix degradation, and is also involved in tissue remodeling, fibrosis, wound healing, inflammation, and tumor growth. FAP exists in both plasma membrane and soluble forms.

In some embodiments, the support cells are modified to overexpress the coding sequence of a FAP protein. In some embodiments, the support cells are genetically modified and carry stable integrations of one or more copies of a FAP coding sequence. In some embodiments, the support cells overexpress the coding sequence of FAP protein at levels sufficient to increase expression of a FAP at the surface of the cell.

In some embodiments, the FAP coding sequence is selected from any metazoan species. In some embodiments, the FAP coding sequence is from any animal, such as vertebrate and invertebrate animal species. In some embodiments, the FAP coding sequence is from any vertebrate animal species such as mammals, reptiles, birds, amphibians, and the like. In some embodiments, the FAP coding sequence is from any mammalian species such as a human, murine, bovine, porcine, poultry, and the like. In some embodiments, the coding sequence of the FAP protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of FAP may be achieved using different approaches. In some embodiments, the expression is inducible. In some embodiments, the method comprises expressing polynucleotides comprising the coding sequence of FAP. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the FAP gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of FAP may encode any homolog of FAP, including FAP paralogs, such as APEH, DPP4, DPP6, DPP8, DPP9, and DPP10, or any other FAP paralogs, or a FAP protein translated from any splice variants of a FAP gene, or may comprise any mutations in the FAP gene sequence including, but not limited to nucleotide deletions, truncations, fusions, or substitutions. Mutations may be synthetic or naturally occurring.

In some embodiments, FAP refers to the Fap gene or FAP protein, or fragment or variant thereof (e.g., a FAP protein having one or more (e.g., one, two, three, four, five, six, seven, eight, nine, ten, or more amino acid substitutions, deletions or insertions as compared to a wild type FAP protein)).

In some embodiments, an FAP protein comprises an amino acid sequence having at least 80% ((e.g., at least 85%, 90%, 95%, 96%, 97%, 98%, or 99%) sequence identity to a sequence selected from SEQ ID NOs: 1-11. In some embodiments, the FAP protein sequence comprises an amino acid sequence selected from SEQ ID NOs: 1-11.

### Insulin Like Growth Factor

In some embodiments, the methods provided herein include a support cell comprising a polynucleotide comprising a coding sequence of insulin like growth factor 2 (IGF2) or a fragment thereof. As used herein, “IGF2” refers to the insulin like growth factor 2 (Igf2) gene or IGF2 protein that is a member of the insulin like growth factor family (e.g., growth factor ligand).

Without wishing to be bound by theory, IGF2 acts as a ligand for insulin-like growth factor 2 receptor (IGF2R). IGF2 can regulate myogenic transcription factor MYOD1 function by facilitating the recruitment of transcriptional coactivators, thereby controlling muscle terminal differentiation. In some cases, IGF2 inhibits myoblast differentiation and modulates metabolism via increasing the mitochondrial respiration rate.

In some embodiments, the support cells are modified to overexpress the coding sequence of an IGF2 protein. In some embodiments, the support cells are genetically modified and carry stable integrations of one or more copies of an IGF2 coding sequence. In some embodiments, the support cells overexpress the coding sequence of IGF2 protein at levels sufficient to increase production and/or secretion of IGF2 into the cell medium.

In some embodiments, the IGF2 coding sequence is selected from any metazoan species. In some embodiments, the IGF2 coding sequence is from any animal, such as vertebrate and invertebrate animal species. In some embodiments, the IGF2 coding sequence is from any vertebrate animal species such as mammals, reptiles, birds, amphibians, and the like. In some embodiments, the IGF2 coding sequence is from any mammalian species such as a human, murine, bovine, porcine, poultry, and the like. In some embodiments, the coding sequence of the IGF2 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of IGF2 may be achieved using different approaches. In some embodiments, the expression is inducible. In some embodiments, the method comprises expressing polynucleotides comprising the coding sequence of IGF2. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the IGF2 gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of IGF2 may encode any homolog of IGF2, including IGF2 paralogs, such as IGF1, INS, and INS-IGF2, or any other IGF2 paralogs, or an IGF2 protein translated from any splice variants of an IGF2 gene, or may comprise any mutations in the IGF2 gene sequence including, but not limited to nucleotide deletions, truncations, fusions, or substitutions. Mutations may be synthetic or naturally occurring.

In some embodiments, IGF2 refers to the Igf2 gene or IGF2 protein, or fragment or variant thereof (e.g., a IGF2 protein having one or more (e.g., one, two, three, four, five, six, seven, eight, nine, ten, or more amino acid substitutions, deletions or insertions as compared to a wild type IGF2 protein)).

In some embodiments, an IGF2 protein comprises an amino acid sequence having at least 80% ((e.g., at least 85%, 90%, 95%, 96%, 97%, 98%, or 99%) sequence identity to a sequence selected from SEQ ID NOs: 12-44. In some embodiments, the IGF2 protein sequence comprises an amino acid sequence selected from SEQ ID NOs: 12-44.

### SDC4

In some embodiments, the methods provided herein include a cell comprising a polynucleotide comprising a coding sequence of Syndecan 4 (SDC4) or a fragment thereof. As used herein, “SDC4” refers to the syndecan 4 (Sdc4) gene or SDC4 protein. SDC4 is a transmembrane (type I) heparan sulfate proteoglycan that functions as a receptor in intracellular signaling.

In some embodiments, the support cells are modified to overexpress the coding sequence of an SDC4 protein. In some embodiments, the support cells are genetically modified and carry stable integrations of one or more copies of an SDC4 coding sequence. In some embodiments, the support cells overexpress the coding sequence of SDC4 protein at levels sufficient to increase expression of SDC4 at the surface of the cell.

In some embodiments, the SDC4 coding sequence is selected from any metazoan species. In some embodiments, the SDC4 coding sequence is from any animal, such as vertebrate and invertebrate animal species. In some embodiments, the SDC4 coding sequence is from any vertebrate animal species such as mammals, reptiles, birds, amphibians, and the like. In some embodiments, the SDC4 coding sequence is from any mammalian species such as a human, murine, bovine, porcine, poultry, and the like. In some embodiments, the coding sequence of the SDC4 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of SDC4 may be achieved using different approaches. In some embodiments, the expression is inducible. In some embodiments, the method comprises expressing polynucleotides comprising the coding sequence of SDC4. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the SDC4 gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of SDC4 may encode any homolog of SDC4, including SDC4 paralogs, such as SDC1, SDC2, SDC3, or any other SDC4 paralogs, or an SDC4 protein translated from any splice variants of an SDC4 gene, or may comprise any mutations in the SDC4 gene sequence including, but not limited to nucleotide deletions, truncations, fusions, or substitutions. Mutations may be synthetic or naturally occurring.

In some embodiments, SDC4 refers to the Sdc4 gene or SDC4 protein, or fragment or variant thereof (e.g., a SDC4 protein having one or more (e.g., one, two, three, four, five, six, seven, eight, nine, ten, or more amino acid substitutions, deletions or insertions as compared to a wild type SDC4 polypeptide)).

In some embodiments, a SDC4 protein comprises an amino acid sequence having at least 80% ((e.g., at least 85%, 90%, 95%, 96%, 97%, 98%, or 99%) sequence identity to a sequence selected from SEQ ID NOs: 45-57. In some embodiments, the SDC4 protein sequence comprises an amino acid sequence selected from SEQ ID NOs: 45-57.

### SPHK1

In some embodiments, the methods provided herein include a support cell comprising a polynucleotide comprising a coding sequence of sphingosine kinase 1 (SPHK1) or a fragment thereof. As used herein, “SPHK1” refers to the sphingosine kinase 1 (Sphk1) gene or SPHK1 protein sphingosine kinase family. Without wishing to be bound by theory, SPHK1 catalyzes the phosphorylation of sphingosine to form sphingosine-1-phosphate (SiP), a lipid mediator with both intra- and extracellular functions. Intracellularly, SiP regulates proliferation and survival, and extracellularly, it is a ligand for cell surface G protein-coupled receptors. In some cases, SPHK1, and its product SIP, play a key role in TNF-alpha signaling and the NF-kappa-B activation pathway important in inflammatory, antiapoptotic, and immune processes. In some cases, phosphorylation of SPHK1 alters its catalytic activity and promotes its translocation to the plasma membrane.

In some embodiments, the support cells are modified to overexpress the coding sequence of an SPHK1 protein. In some embodiments, the support cells are genetically modified and carry stable integrations of one or more copies of an SPHK1 coding sequence. In some embodiments, the support cells overexpress the coding sequence of SPHK1 protein at levels sufficient to increase production and/or secretion of SPHK1 into the cell medium. In some embodiments, the cells overexpress the coding sequence of SPHK1 protein at levels sufficient to increase expression of SPHK1 at the surface of the cells.

In some embodiments, the SPHK1 coding sequence is selected from any metazoan species. In some embodiments, the SPHK1 coding sequence is from any animal, such as vertebrate and invertebrate animal species. In some embodiments, the SPHK1 coding sequence is from any vertebrate animal species such as mammals, reptiles, birds, amphibians, and the like. In some embodiments, the SPHK1 coding sequence is from any mammalian species such as a human, murine, bovine, porcine, poultry, and the like. In some embodiments, the coding sequence of the SPHK1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of SPHK1 may be achieved using different approaches. In some embodiments, the expression is inducible. In some embodiments, the method comprises expressing polynucleotides comprising the coding sequence of SPHK1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the SPHK1 gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of SPHK1 may encode any homolog of SPHK1, including SPHK1 paralogs, such as SPHK1, or any other SPHK1 paralogs, or an SPHK1 protein translated from any splice variants of an SPHK1 gene, or may comprise any mutations in the SPHK1 gene sequence including, but not limited to nucleotide deletions, truncations, fusions, or substitutions. Mutations may be synthetic or naturally occurring.

In some embodiments, SPHK1 refers to the Sphk1 gene or SPHK1 protein, or fragment or variant thereof (e.g., a SPHK1 protein having one or more (e.g., one, two, three, four, five, six, seven, eight, nine, ten, or more amino acid substitutions, deletions or insertions as compared to a wild type SPHK1 polypeptide)).

In some embodiments, a SPHK1 protein comprises an amino acid sequence having at least 80% ((e.g., at least 85%, 90%, 95%, 96%, 97%, 98%, or 99%) sequence identity to a sequence selected from SEQ ID NOs: 58-83. In some embodiments, the SPHK1 protein sequence comprises an amino acid sequence selected from SEQ ID NOs: 58-83.

### Focal Adhesion Kinase

In some embodiments, the methods provided herein include a support cell comprising a polynucleotide comprising a coding sequence of focal adhesion kinase (FAK) or a fragment thereof. Aliases for FAK include protein tyrosine kinase (PTK2). As used herein, “FAK” refers to the focal adhesion kinase 1 (Fak) gene or FAK, which is a member of the FAK subfamily of protein tyrosine kinases. FAK is a non-receptor protein-tyrosine kinase that plays an essential role in regulating cell migration, adhesion, spreading, reorganization of the actin cytoskeleton, formation and disassembly of focal adhesions and cell protrusions, cell cycle progression, cell proliferation and apoptosis.

In some embodiments, the support cells are modified to overexpress the coding sequence of an FAK protein. In some embodiments, the support cells are genetically modified and carry stable integrations of one or more copies of an FAK coding sequence. In some embodiments, the support cells overexpress the coding sequence of FAK protein at levels sufficient to increase production and/or secretion of FAK into the cell medium.

In some embodiments, the FAK coding sequence is selected from any metazoan species. In some embodiments, the FAK coding sequence is from any animal, such as vertebrate and invertebrate animal species. In some embodiments, the FAK coding sequence is from any vertebrate animal species such as mammals, reptiles, birds, amphibians, and the like. In some embodiments, the FAK coding sequence is from any mammalian species such as a human, murine, bovine, porcine, poultry, and the like. In some embodiments, the coding sequence of the FAK protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, increasing expression of FAK may be achieved using different approaches. In some embodiments, the expression is inducible. In some embodiments, the method comprises expressing polynucleotides comprising the coding sequence of FAK. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the FAK gene involves electroporating a DNA, delivering a DNA complexed with a transfection vehicle, using a viral vector (e.g. retrovirus, lentivirus, adenovirus, adeno-associated virus), and the like, or combinations thereof. In some embodiments, the expression is constitutive. In some embodiments, the expression is conditional (e.g. inducible).

In the methods described herein, a polynucleotide comprising a coding sequence of FAK may encode any homolog of FAK, including FAK paralogs, such as PTK2B, ABL1, and ABL2,or any other FAK paralogs, or an FAK protein translated from any splice variants of an FAK gene, or may comprise any mutations in the FAK gene sequence including, but not limited to nucleotide deletions, truncations, fusions, or substitutions. Mutations may be synthetic or naturally occurring.

In some embodiments, FAK refers to the Fak gene or FAK protein, or fragment or variant thereof (e.g., a FAK protein having one or more (e.g., one, two, three, four, five, six, seven, eight, nine, ten, or more amino acid substitutions, deletions or insertions as compared to a wild type FAK protein)).

In some embodiments, a FAK protein comprises an amino acid sequence having at least 80% ((e.g., at least 85%, 90%, 95%, 96%, 97%, 98%, or 99%) sequence identity to a sequence selected from SEQ ID NOs: 84-96. In some embodiments, the FAK protein sequence comprises an amino acid sequence selected from SEQ ID NOs: 84-96.

In some embodiments, the FAK protein is a wild type chicken FAK (SEQ ID NO: 1). In some embodiments, the FAK is a stabilized version of wild type chicken FAK (SEQ ID NO: 2). In such embodiments, the FAK comprises one or more amino acid substitutions engineered to impart increased thermostability on the FAK protein (i.e., increase half-life in the culture medium).

### 6.4 Method for Increasing Bioconversion Efficiency (BCE) of a Myogenic Cell Using Co-Culture

Provided herein are methods for increasing bioconversion efficiency (BCE) of a myogenic cells comprising: (a) co-culturing a myogenic cell with a support cell, wherein the support cell line comprises a polynucleotide comprising a coding sequence of a gene of interest; and (b) culturing the myogenic cell and the support cell in a cultivation infrastructure under conditions sufficient to induce formation of the myocytes and multinucleated myotubes from the myogenic cell. In such cases, bioconversion efficiency refers to the efficiency by which a myogenic cell differentiates into a myocyte, multinucleated myotube and/or skeletal muscle tissue.

In some embodiments, an increase in the bioconversion efficiency of a myogenic cell using the co-culture methods described herein is about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 2-fold, 2.5-fold, 3-fold, 3.5-fold, 4-fold, 4.5-fold, 5-fold, 7.5-fold, 10-fold, 15-fold, 20-fold, 25-fold, 30-fold, 40-fold, or even about 50-fold, 75-fold, 100-fold, 150-fold, or about 200-fold as compared to the bioconversion efficiency of a myogenic cell that is either co-cultured with a support cell line not engineered to express a gene of interest or not co-cultured with a support cell line.

In some embodiments, an increase in the bioconversion efficiency of a myogenic cell using a co-culture methods described herein is at least 10%, at least 15%, at least 20%, at least 25%, at least 30%, at least 35%), at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, compared to the bioconversion efficiency of a myogenic cell that is either co-cultured with a support cell line not engineered to express a gene of interest or not co-cultured with a support cell line.

In some embodiments, methods described herein increase the bioconversion efficiency of a myogenic cell using a co-culture by increasing the rate of proliferation of cells in the culture. In some embodiments, the increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%), including values and ranges therebetween, compared to the bioconversion efficiency of a myogenic cell that is either co-cultured with a support cell line not engineered to express a gene of interest or not co-cultured with a support cell line. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to the bioconversion efficiency of a myogenic cell that is either co-cultured with a support cell line not engineered to express a gene of interest or not co-cultured with a support cell line.

### 6.5 Method of Increasing Cell Density Using Co-Culture

Provided herein are methods for increasing the cell density of a culture comprising a myogenic cell line where the method includes (a) co-culturing a myoblast myogenic cell line with a support cell line, wherein the support cell comprises a polynucleotide comprising a coding sequence of a gene of interest; and (b) culturing the myoblast myogenic cell and the support cell in a cultivation infrastructure under conditions sufficient to induce proliferation of the myogenic cell. Provided herein are methods for increasing the cell density of a culture comprising a myogenic cell line where the method includes (a) co-culturing a myoblast myogenic cell line with a support cell line, wherein the myogenic cell comprises a polynucleotide comprising a coding sequence of a gene of interest, and wherein the support cell comprises a polynucleotide comprising a coding sequence of a second gene of interest; and (b) culturing the myoblast myogenic cell and the support cell in a cultivation infrastructure under conditions sufficient to induce proliferation of the myogenic cell.

In some embodiments, an increase in the cell density of a population of myogenic cells in a culture (e.g., an adherent culture or a suspension culture) using the methods described herein is about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 2-fold, 2.5-fold, 3-fold, 3.5-fold, 4-fold, 4.5-fold, 5-fold, 7.5-fold, 10-fold, 15-fold, 20-fold, 25-fold, 30-fold, 40-fold, or even about 50-fold, 75-fold, 100-fold, 150-fold, or about 200-fold, compared to the density of a population of myogenic cells in a culture that is either co-cultured with a support cell line not engineered to express a gene of interest or not co-cultured with a support cell line.

In some embodiments, an increase in the density of a population of myogenic cells in a culture (e.g., an adherent culture or a suspension culture) using the methods described herein is at least 10%, at least 15%, at least 20%, at least 25%, at least 30%, at least 35%), at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, compared to the density of a population of myogenic cells in a culture that is either co-cultured with a support cell line not engineered to express a gene of interest or not co-cultured with a support cell line.

In some embodiments, methods described herein increase the density of a population myogenic cells in a culture (e.g., an adherent culture or a suspension culture) by increasing the rate of proliferation of cells in the culture. In some embodiments, the increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%), including values and ranges therebetween, compared to the density of a population of myogenic cells in a culture that is either co-cultured with a support cell line not engineered to express a gene of interest or not co-cultured with a support cell line. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to the density of a population of myogenic cells in a culture that is either co-cultured with a support cell line not engineered to express a gene of interest or not co-cultured with a support cell line.

In some embodiments, methods described herein increase the cell density of a population of myogenic cells in a culture (e.g., an adherent culture or a suspension culture) by decreasing cell death within the cellular biomass. In some embodiments, the decrease in cell death is at least 2.5%, at least 5%, at least 10%>, at least 15%, at least 20%, at least 25%, at least 30%), at least 35%, at least 40%, at least 45%, at least 50%, at least 55%, at least 60%, at least 65%, at least 70%, at least 75%, at least 80%, at least 85%, at least 90%, at least 95%, at least 100%), including values and ranges therebetween, compared to the density of a population of myogenic cells in a culture that is either co-cultured with a support cell line not engineered to express a gene of interest or not co-cultured with a support cell line. In some embodiments, a decrease in the rate of cell death within the population of myogenic cells is about 20.5-10%, about 20.5-75%, about 20.5-50%, about 5.0-100%, about 5.0-75%, about 5.0-50%, about 10-100%, about 10-75%, or about 10-50%, including values and ranges therebetween, compared to the density of a population of myogenic cells in a culture that is either co-cultured with a support cell line not engineered to express a gene of interest or not co-cultured with a support cell line.

In some embodiments, using the methods described herein, the density of a population of myogenic cells in a culture may reach about 1E4 cells/mL, about 1E5 cells/mL, about 1E6 cells/mL, about 1E7 cells/mL, about 1E8 cells/mL, about 1E9 cells/mL, about 1E10 cells/mL, about 1E11 cells/mL, about 1E12 cells/mL, or about 1E13 cells/mL (cells in suspension culture or cells in the myogenic cells/mL of cultivation infrastructure), including values and ranges therebetween.

In some embodiments, using the methods described herein, the density of a population of myogenic cells in a culture (e.g., suspension culture) may reach about 1 g/L, 5 g/L, 10 g/L, 25 g/L, 50 g/L, 75 g/L, 100 g/L, 150 g/L, 200 g/L, 250 g/L, 300 g/L, 350 g/L, 400 g/L, 450 g/L, 500 g/L, 550 g/L, 600 g/L, 650 g/L, 700 g/L, 750 g/L, 800 g/L, 850 g/L, 900 g/L, or 1000 g/L (g of myogenic cells/L of cultivation infrastructure), including values and ranges therebetween. In some embodiments, the density of a population of myogenic cells in a culture (e.g., an adhesion culture or a suspension culture) may range from about 1 g/L to about 5 g/L, about 1 g/L to about 750 g/L, about 1 g/L to about 500 g/L, about 1 g/L to about 250 g/L, about 1 g/L to about 100 g/L, about 1 g/L to about 50 g/L, about 5 g/L to about 1000 g/L, about 5 g/L to about 750 g/L, about 5 g/L to about 500 g/L, about 5 g/L to about 250 g/L, about 5 g/L to about 100 g/L, about 5 g/L to about 50 g/L, about 25 g/L to about 1000 g/L, about 25 g/L to about 750 g/L, about 25 g/L to about 500 g/L, about 25 g/L to about 300 g/L, about 25 g/L to about 250 g/L, about 25 g/L to about 100 g/L, about 50 g/L to about 1000 g/L, about 50 g/L to about 750 g/L, about 50 g/L to about 500 g/L, about 50 g/L to about 300 g/L, about 50 g/L to about 250 g/L, about 100 g/L to 1000 g/L, about 100 g/L to about 750 g/L, about 100 g/L to about 500 g/L, about 200 g/L to about 1000 g/L, about 200 g/L to about 750 g/L, about 200 g/L to about 500 g/L, about 300 g/L to about 1000 g/L, about 300 g/L to about 800 g/L, about 400 g/L to about 1000 g/L, or about 500 g/L to about 1000 g/L including values and ranges therebetween.

### 6.6 Method for Increasing Myotube Formation from a Myogenic Cell

Provided herein are methods for increasing myotube formation from a myogenic cell, comprising (a) co-culturing a myogenic cell with a support cell, wherein the support cell comprises a polynucleotide comprising a coding sequence of a gene of interest; and (b) culturing the myogenic cell and the support cell in a cultivation infrastructure under conditions sufficient to induce proliferation of the myoblast cell. Provided herein are methods for increasing myotube formation from a myogenic cell, comprising (a) co-culturing a myogenic cell with a support cell, wherein the myogenic cell comprises a polynucleotide comprising a coding sequence of a gene of interest, and wherein the support cell comprises a polynucleotide comprising a coding sequence of a gene of interest; and (b) culturing the myogenic cell and the support cell in a cultivation infrastructure under conditions sufficient to induce myotube formation from the myogenic cell.

In some embodiments, a myogenic cell line's ability to form myotubes can be modulated by co-culturing the myogenic cell line with a support cell line comprising a polynucleotide comprising a coding sequence of a gene of interest. In some embodiments, a myogenic cell line's ability to form myotubes can be modulated by introducing in the myogenic cell a polynucleotide comprising a coding sequence of a first gene of interest and co-culturing the myogenic cell line with a support cell line comprising a polynucleotide comprising a coding sequence of a second gene of interest.

In some embodiments, co-culturing a myogenic cell with a support cell, where the support cell comprises a polynucleotide comprising a coding sequence of a gene of interest results in increased myotube formation as compared to myotube formation for a population of myogenic cells in a culture that is either co-cultured with a support cell line not engineered to express a gene of interest or not co-cultured with a support cell line. In some embodiments, co-culturing a myogenic cell with a support cell, where the support cell comprises a polynucleotide comprising a coding sequence of a gene of interest results in an increase in myotube formation of at least 2.5%, at least 5%, 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, including values and ranges therebetween, as compared to myotube formation for a population of myogenic cells in a culture that is either co-cultured with a support cell line not engineered to express a gene of interest or not co-cultured with a support cell line.

In some embodiments, co-culturing a myogenic cell with a support cell, where the myogenic cell comprises a polynucleotide encoding a first gene of interest and the support cell comprises a polynucleotide comprising a coding sequence of a second gene of interest results in increased myotube formation as compared to myotube formation for a population of myogenic cells in a culture that is either co-cultured with a support cell line not engineered to express a gene of interest or not co-cultured with a support cell line. In some embodiments, co-culturing a myogenic cell with a support cell, where the myogenic cell comprises a polynucleotide comprising a coding sequence of a first gene of interest and a support cell comprises a polynucleotide comprising a coding sequence of a gene of interest results in an increase in myotube formation of at least 2.5%, at least 5%, 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, including values and ranges therebetween, as compared to myotube formation for a population of myogenic cells in a culture that is either co-cultured with a support cell line not engineered to express a gene of interest or not co-cultured with a support cell line.

In some embodiments, co-culturing a myogenic cell with a support cell, where the support cell comprises a polynucleotide comprising a coding sequence of a gene of interest results in an increased percentage of myogenic cells exhibiting MyHC1 expression as compared to myotube formation for a population of myogenic cells in a culture that is either co-cultured with a support cell line not engineered to express a gene of interest or not co-cultured with a support cell line. In some embodiments, co-culturing a myogenic cell with a support cell, where the myogenic cell comprises a polynucleotide comprising a coding sequence of a first gene of interest and the support cell comprises a polynucleotide comprising a coding sequence of a second gene of interest results in an increased percentage of myogenic cells exhibiting MyHC1 expression as compared to myotube formation for a population of myogenic cells in a culture that is either co-cultured with a support cell line not engineered to express a gene of interest or not co-cultured with a support cell line. In some embodiments, the population of myogenic cells produced as a result of the co-culturing comprise at least 50% (e.g., at least 55%, at least 60%, at least 65%, at least 70%, at least 75%, at least 80%, at least 85%, at least 90%, at least 95%, at least 99%, or 100%) MyHC⁺ cells.

In some embodiments, co-culturing a myogenic cell with a support cell, where the support cell comprises a polynucleotide comprising a coding sequence of a gene of interest results in an increased percentage of myogenic cells exhibiting Myogenin expression as compared to myotube formation for a population of myogenic cells in a culture that is either co-cultured with a support cell line not engineered to express a gene of interest or not co-cultured with a support cell line. In some embodiments, co-culturing a myogenic cell with a support cell, where the myogenic cell comprises a polynucleotide comprising a coding sequence of a first gene of interest and the support cell comprises a polynucleotide comprising a coding sequence of a second gene of interest results in an increased percentage of myogenic cells exhibiting Myogenin expression as compared to myotube formation for a population of myogenic cells in a culture that is either co-cultured with a support cell line not engineered to express a gene of interest or not co-cultured with a support cell line. In some embodiments, the population of myogenic cells produced as a result of the co-culturing comprise at least 50% (e.g., at least 55%, at least 60%, at least 65%, at least 70%, at least 75%, at least 80%, at least 85%, at least 90%, at least 95%, at least 99%, or 100%) Myogenin⁺ cells.

### 6.7 Methods for Producing Cell-Based Meat Suitable for Consumption

Provided herein are in vitro methods for producing cell-based meat suitable for consumption, comprising: (a) co-culturing a myogenic cell and a support cell, wherein the support cell comprises a polynucleotide comprising a coding sequence of a gene of interest; (b) inducing myogenic specific differentiation, wherein the myogenic cells form myocytes and multinucleated myotubes; and (c) culturing the myocytes and multinucleated myotubes to generate skeletal muscle fibers; and (d) isolating the skeletal muscle fibers and producing the cell based meat product suitable for consumption. In some embodiments, the myogenic cells include any of the myogenic cells described herein (see Section 4.2.1). In some embodiments, the support cells include any of the support cells described herein (see Section 4.2.2).

In some embodiments, the in vitro method for producing cell-based meat suitable for consumption includes a step of adapting the cell to grown in suspension. In some embodiments, the in vitro method for producing cell-based meat suitable for consumption includes a step of culturing the cells in a cultivation infrastructure. In some embodiments, provided herein is cell-based meat suitable for consumption produced by the in vitro methods described herein.

In some embodiments, the cell line is from a livestock, poultry, game or aquatic animal species. In some embodiments, the cell line is from a chicken, duck, or turkey. In some embodiments, the cell line is from a fish. In some embodiments, the cell line is from a livestock species. In some embodiments, the livestock species is porcine or bovine. In some embodiments, the cells are from any animal species intended for human or non-human dietary consumption. In some embodiments, the cells are myogenic cells. In some embodiments, the myogenic cells are myoblasts, myocytes, satellite cells, side population cells, muscle derived stem cells, mesenchymal stem cells, myogenic pericytes, or mesangioblasts. In some embodiments, the cells are non-myogenic cells.

Non-limiting examples of myogenic differentiation are as described in WO2019014652A1 and WO2015066377A1, both of which are herein incorporated by reference in their entireties.

In some embodiments, the skeletal muscle produced according to the methods described herein can be processed as a raw, uncooked food product (cultured meat) or as a cooked food product or as a cooked/uncooked food ingredient. In some embodiments, processing comprises withdrawal of the culture medium that supports the viability, survival, growth or expansion (e.g., increase in total protein content of the non-naturally occurring myogenic cells) and differentiation of the myogenic cells. Withdrawal may comprise physical removal of the culture medium or altering the composition of the culture medium, for example, by addition of components that would reduce or prevent further expansion and/or differentiation of the cell line or cells-derived from the cell line or by depletion of components that support expansion and/or differentiation of the cell line or cells derived from the cell line.

### 6.8 Cell-Based Meat Product

Provided herein is a cell-based meat product suitable for consumption comprising a myogenic cell (e.g., any of the myogenic cells described (see Section 4.2.1) and a support cell (e.g., any of the support cells described herein (see Section 4.2.2).

In some embodiments, the cell-based meat product has various characteristics. Exemplary characteristics of the cell-based meat are described in U.S. application Ser. No. 17/033,635 and PCT Application No. PCT/US2021/016681, which are herein incorporated by reference in their entireties. In some embodiments, the cell-based meat product of the disclosure can be modified to achieve certain textual features, such as a desired cooked bite force or cooked hardness. See Table 17 of PCT US2021/016681 for the cooked texture of exemplary cell-based meat samples.

### 6.9 Cultivation Infrastructure

In some embodiments, a cultivation infrastructure may be a tube, a cylinder, a flask, a petri-dish, a multi-well plate, a dish, a vat, a roller bottle, an incubator, a bioreactor, an industrial fermenter and the like.

In some embodiments, a cultivation infrastructure can be of any scale, and support any volume of cellular biomass and culturing reagents. In some embodiments, the cultivation infrastructure ranges from about 10 μL to about 100,000 μL. In some embodiments, the cultivation infrastructure is about 10 μL, about 100 μL, about 1 mL, about 10 mL, about 100 mL, about 1 μL, about 10 μL, about 100 μL, about 1000 μL, about 10,000 μL, or even about 100,000 μL.

In some embodiments, the cultivation infrastructure comprises a substrate. In some embodiments, a cultivation infrastructure may comprise a permeable substrate (e.g. permeable to physiological solutions) or an impermeable substrate (e.g. impermeable to physiological solutions).

In some embodiments, the cultivation infrastructure comprises a primary substrate, which can be a flat, concave, or convex substrate. In some embodiments, the cultivation infrastructure further comprises a secondary substrate, either introduced, or autologous, to direct cellular growth between the substrates, e.g. to direct attachment, proliferation and hypertrophy of cells on a plane perpendicular to the primary substrate.

In some embodiments, the cultivation infrastructure comprises a hydrogel, a liquid cell culture media, or soft agar.

In some embodiments, the cultivation infrastructure does not comprise a substrate to which cells can adhere. In some embodiments, the cultivation infrastructure comprises a suspension culture, e.g. supporting the growth of a self-adhering biomass, or single-cell suspension in a liquid medium.

In some embodiments, the cultivation infrastructure comprises adherent cells (i.e. those cells that adhere to a substrate). In some embodiments, the cultivation infrastructure comprises non-adherent cells (i.e. those cells that do not adhere to a substrate). In some embodiments, the cultivation infrastructure comprises both adherent and non-adherent cells.

### 6.10 Immortalization

In some embodiments, the method provided herein include a cell line immortalized prior, contemporaneously therewith, or after introducing into the cell any of the polynucleotides described herein.

In some embodiments, immortalization comprises transforming a cell with a telomerase reverse transcriptase (TERT) gene. As used herein, “TERT” refers to telomerase reverse transcriptase (TERT) gene or TERT polypeptide that is a ribonucleoprotein polymerase that maintains telomere ends by addition of the telomere repeat TTAGGG. Telomerase expression plays a role in cellular senescence, as it is normally repressed in postnatal somatic cells resulting in progressive shortening of telomeres. In some embodiments, cells ectopically express the TERT polynucleotide. In some embodiments, the cells are genetically modified and carry stable integrations of one or more copies of the TERT polynucleotide. Exemplary methods for immortalizing a cell line are as described in WO2019014652A1, which is herein incorporated by reference in its entirety.

In some embodiments, increased expression of TERT may be achieved using different approaches. In some embodiments, increased expression of TERT may be achieved by ectopically expressing TERT. In some embodiments, increased expression of TERT may be achieved by introducing targeted mutations in the TERT promoter. In some embodiments, increased expression of TERT may be achieved by activating endogenous TERT expression by an engineered transcriptional activator. In some embodiments, increased expression of TERT may be achieved by transiently transfecting TERT mRNA.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any mammalian species, such as a human, murine, bovine, porcine, and the like.

In some embodiments, immortalization comprises transforming a cell with a polynucleotide encoding a cyclin-dependent kinase 4 (“CDK4”) protein. In some embodiments, immortalization comprises inactivating a gene encoding an inhibitor of cyclin-dependent kinase 4 (CDK4). Exemplary methods for immortalizing a cell line are as described in WO2017124100A1, which is herein incorporated by reference in its entirety.

### 6.11 Nucleic Acids/Vectors

Also provided herein are polynucleotides comprising coding sequences of any of the genes of interest described herein. In some embodiments, the polynucleotides includes sequences having at least 80% (e.g., at least 85%, 90%, 95%, 96%, 97%, 98%, or 99%) sequence identity to a sequence selected from SEQ ID NOs: 97-101.

Also provided herein is a construct (i.e., a vector) that includes any of the polynucleotides described herein. In some embodiments, any of the vectors described herein can be an expression vector. In some embodiments, an expression vector can include one or more promoter sequences (e.g., any of the promoter sequences described herein) operably linked to a coding sequence of any of the genes of interest described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some embodiments, a vector includes sufficient cis-acting elements that supplement expression where the remaining elements needed for expression can be supplied by the host cell (e.g., the cell line).

In some embodiments, a vector includes a polynucleotide comprising a coding sequence of a single gene of interest or fragment thereof. In some embodiments, a vector includes a polynucleotide comprising a first coding sequence of a first gene of interest and a second coding sequence of a second gene of interest. In some embodiments, a vector (e.g., a construct) includes a polynucleotide comprising a first coding sequence of a first gene of interest, a second coding sequence of a second gene of interest, and a third coding sequence of a third gene of interest. In such embodiments where the construct includes coding sequences for two or more gene of interest, each of the two or more coding sequences are operably linked to a promoter sequence or to another coding sequence via a self-cleaving polypeptide or IRES. As used herein, the term “operably linked” is well known in the art and refers to genetic components that are combined such that they carry out their normal functions. For example, a coding sequence is operably linked to a promoter when its transcription is under the control of the promoter. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

In some embodiments, a coding sequence of any one or more of the genes of interest described herein is operably linked to a promoter. In some embodiments, the promoter is a tissue-specific promoter. In some embodiments, the tissue-specific promoter is a muscle-specific promoter. In some embodiments, the muscle-specific promoter is selected from the group consisting of: skeletal β-action, myosin light chain 2a, dystrophin, SPc-512, muscle creatine kinase, and synthetic muscle promoters. In some embodiments, the promoter is a constitutively active promoter. In some embodiments, the promoter is selected from the group consisting of: EF1 (e.g., EF1alpha), PGK, CMV, RSV, GAPDH and β-actin. In some embodiments, the promoter is a EF1 (e.g., EF1alpha) promoter. In some embodiments, the promoter is a PGK promoter. In some embodiments, the promoter is a GAPDH promoter. In some embodiments, the promoter sequences is derived from Gallus gallus. For example, SEQ ID NOs: 97 and 98 include EF1alpha promoter sequences derived from Gallus gallus and SEQ ID NOs: 99-101 include GAPDH promoter sequences derived from Gallus gallus.

In some embodiments, a vector comprises a first polynucleotide comprising a first coding sequence (e.g., a coding sequence of any of the genes of interest described herein) operably linked to a first promoter and a second polynucleotide comprising a second coding sequence (e.g., a coding sequence of any of the genes of interest described herein) operably linked to a second promoter. In some embodiments, the vector comprises a selectable marker.

In some embodiments, a vector system is used to integrate a polynucleotide comprising a coding sequence of any one or more of the genes of interest described herein into the genome of a cell line (e.g., any of the cell lines described herein). In some embodiments, the vector system used for integration is a vector phiC31 Integrase Vector System. Additional non-limiting examples of vectors systems that can be used to integrate a coding sequence of any one or more of the genes of interest described herein into the genome of a cell line (e.g., any of the cell lines described herein) include: a sleeping beauty transposon system (as described in U.S. Pat. No. 7,985,739), a piggyBac transposition system (as described in US20090042297), CRISPR/Cas-mediated knockin, TALEN-mediated knockin, and viral vector-mediated integration. In such embodiments where integration is mediated via a viral vector, non-limiting examples of viral vectors include adenovirus, adeno-associated virus, lentivirus, retrovirus (e.g., a gamma-retrovirus), or sendai virus.

Methods of introducing nucleic acids and expression vectors into a cell (e.g., an immortalized cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, and nanoparticle transfection.

### 6.12 Culture Media

In some embodiments, the methods provided herein include co-culturing the myogenic cells with support cells (e.g., any of the support cells described herein) using a culture media, including a growth media, and/or a differentiation media. For example, co-culturing the myogenic cells with support cells (e.g., any of the support cells described herein) comprises exposing the co-culture to a growth media, a differentiation media, or a growth media and a differentiation media. In such cases, the co-culture can be contacted with a growth media for a first period of time (e.g., any time sufficient to induce growth of the myogenic cells) and then contacted with a differentiation media for a second period of time (e.g., any time sufficient to induce differentiation of the myogenic cells to a phenotype of interest (e.g., skeletal muscle cell).

In some embodiments, co-culturing the myogenic cells with support cells (e.g., any of the support cells described herein) uses edible nutrient medium as described in U.S. Patent Publication No. 2022/0073870, which is herein incorporated by reference in its entirety.

### Growth Media

In some embodiments, a growth media comprises base media without any additional additives. Non-limiting examples of base media include: DMEM/F12, MEM, and IMDM. In some embodiments, a growth media comprises base media including serum. For example, growth media includes about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19%, or about 20% serum. In some embodiments, differentiation media includes about 20% serum. In some embodiments, differentiation media includes about 10% serum. In some embodiments, the growth media is serum free.

In some embodiments, growth media includes serum derived from two or more species. In some embodiments, serum is selected from; fetal bovine serum (FBS), chicken serum, and horse serum, or a combination thereof. In some embodiments, growth media includes FBS and horse serum, FBS and chicken serum, horse serum and chicken serum, or FBS, chicken serum, and horse serum. In some embodiments, growth media includes about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19% or about 20% of a first serum (e.g., FBS) and about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19% or about 20% of a second serum (e.g., chicken serum).

In some embodiments, growth media includes signaling pathway modulators. In some embodiments, the signaling pathways can either be activated or inhibited. For example, without limitation, Wnt, TGF (Activin A), and BMP signaling pathways can be activated (e.g., using CHIR99021, Activin A, or BMP4, respectively) or inhibited (e.g., using IWR1, A-83-01, or LDN193189, respectively). In some embodiments, growth media includes an activator of Wnt signaling (e.g., CHIR99021), an inhibitor of TGF (Activin A) signaling (e.g., A-83-01), and an inhibitor of BMP signaling (e.g., LDN193189).

In some embodiments, the methods described herein can include culture media as described in International Patent Application No. PCT/US2022/082175 filed on Dec. 21, 2022).

In some embodiments, growth media is selected from: (i) DMEM/F12, about 20% FBS, and about 5% chicken serum; (ii) DMEM/F12, about 20% FBS, about 5% chicken serum, CHIR99021, A-83-01, and LDN193189; and (iii) DMEM/F12, about 10% FBS, and about 5% chicken serum.

In some embodiments, a first period of time includes 10, 11, 12, 13, 14, 15, 16, 17, 18, 19, 20, 21, 22, 23, 24, 25, 26, 27, 28, 29 or 30 or more days. In some embodiments, a first period of time includes 1 month, 2 months, 3 months, 4 months, 5 months, 6 months, 7 months, 8 months, 9 months, 10 months, 11 months or 12 months or more.

In some embodiments, a first period of time includes the amount of time needed for the myogenic cells to have a population doubling level (PDL) (i.e., total number of times the cells in the population have doubled since their primary isolation in vitro) of 1, 2, 3, 4, 5, 6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 or more passages.

### Differentiation Media

In some embodiments, a differentiation media comprises base media without any additional additives. Non-limiting examples of base media include: DMEM/F-12, MEM, and IMIDM. In some embodiments, a differentiation media comprises base media including serum (e.g., horse serum). For example, differentiation media includes about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19%, or about 20% serum. In some embodiments, differentiation media includes about 2% serum. In some embodiments, the differentiation media is serum free.

In some embodiments, differentiation media includes serum derived from two or more species. In some embodiments, serum is selected from; fetal bovine serum (FBS), chicken serum, and horse serum, or a combination thereof. In some embodiments, differentiation media includes FBS and horse serum, FBS and chicken serum, horse serum and chicken serum, or FBS, chicken serum, and horse serum. In some embodiments, growth media includes about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19% or about 20% of a first serum (e.g., FBS) and about 0.1%, about 0.5%, about 1.0%, about 2.0%, about 3.0%, about 4%, about 5%, about 6%, about 7%, about 8%, about 9%, about 10%, about 11%, about 12%, about 13%, about 14%, about 15%, about 16%, about 17%, about 18%, about 19% or about 20% of a second serum (e.g., chicken serum).

In some embodiments, differentiation media is selected from: DMEM/F12, about 5% Bovine serum, and about 5% chicken serum; and DMEM/F12, about 2% horse serum, and about 1% ITS (Insulin-Transferrin-Selenium).

### 6.13 Kits

Also provided herein are kits comprising any of the cell lines, any of the cells derived from the cell lines, any of the polynucleotides described herein (e.g., any of the coding sequence of any one or more of the genes of interest described herein). In some embodiments, the kit includes instructions for performing any of the methods described herein.

### 6.14 Additional Processing of Co-Cultured Cells

Provided herein are methods of co-culturing a myogenic cell and a support cell, where the support cell comprises a polynucleotide comprising a coding sequence of a gene of interest, where the co-culture results in production of a cell based meat product suitable for consumption. Once the cell-based meat product suitable for consumption is formed, the methods described herein can be partially or fully terminated and the cell-based meat product suitable for consumption can be harvested.

In some embodiments, the myogenic cells can be differentiated into a phenotype of interest by contacting the cells with a differentiation agent in addition to co-culturing with support cells (e.g., any of the support cells described herein). For example, if the aim of the cell based meat product suitable for consumption is skeletal muscle, the population of myogenic cells can be contacted with a differentiation agent that would induce the skeletal muscle phenotype into the myogenic cells. Exemplary differentiation agents that may induce skeletal muscle phenotype include myogenic transcription factors such as MYOD, MYOG, MYF5, MYF6, PAX3, PAX7, paralogs, orthologs, and genetic variants thereof. Additional non-limiting methods for differentiating myogenic cells into a skeletal muscle phenotype are as described in WO/2015/066377, which is herein incorporated by reference in its entirety. In some embodiments, myogenic cells can be differentiated into a phenotype of interest (e.g., a skeletal muscle cell) without a differentiation agent.

In some embodiments, the cell based meat product can be processed as a raw, uncooked food product (cultured meat) or as a cooked food product or as a cooked/uncooked food ingredient. In some embodiments, processing comprises withdrawal of the culture medium that supports the viability, survival, growth or expansion (e.g., increase in total protein content of the non-natively occurring myogenic cells) and differentiation of the non-natively occurring myogenic cells. Withdrawal may comprise physical removal of the culture medium or altering the composition of the culture medium, for example, by addition of components that would reduce or prevent further expansion and/or differentiation of the population of dedifferentiated cells or population of non-natively occurring myogenic cells or by depletion of components that support expansion and/or differentiation of the population of dedifferentiated cells or population of non-natively occurring myogenic cells.

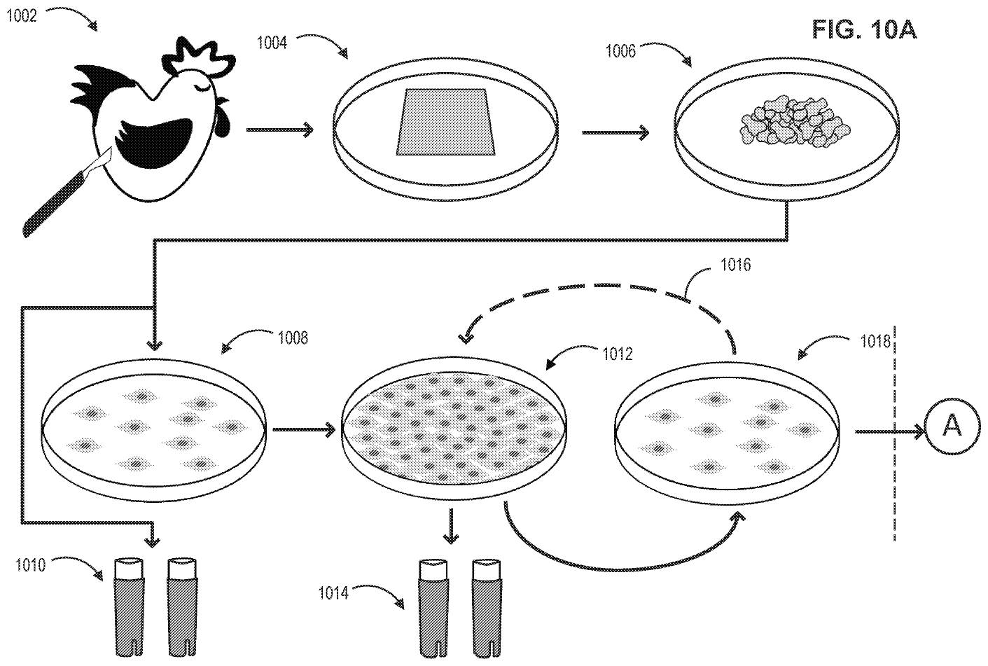

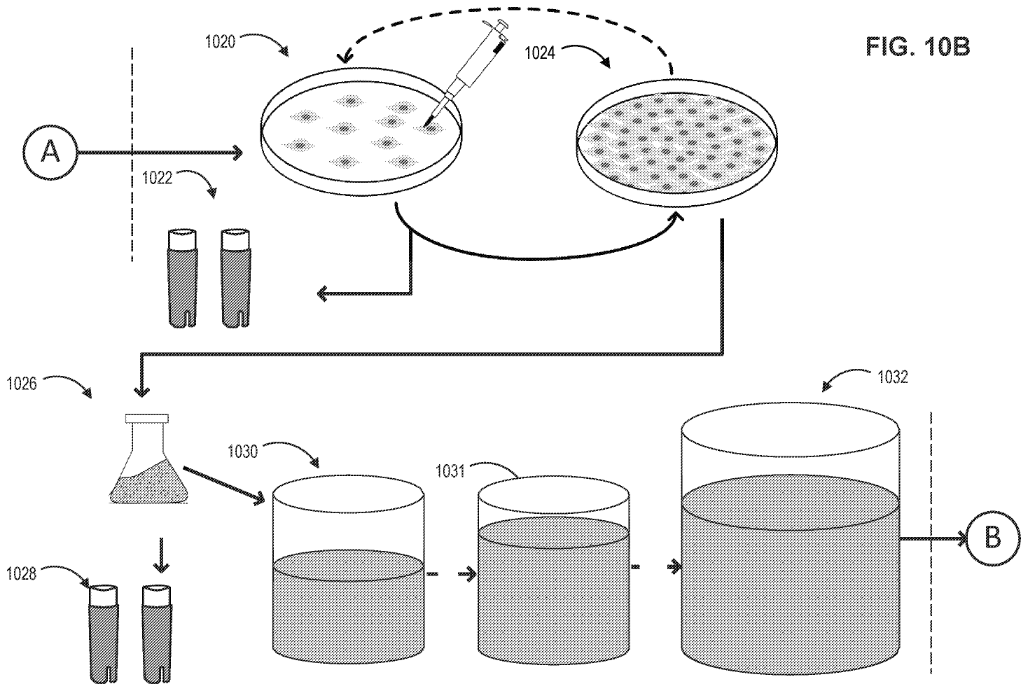

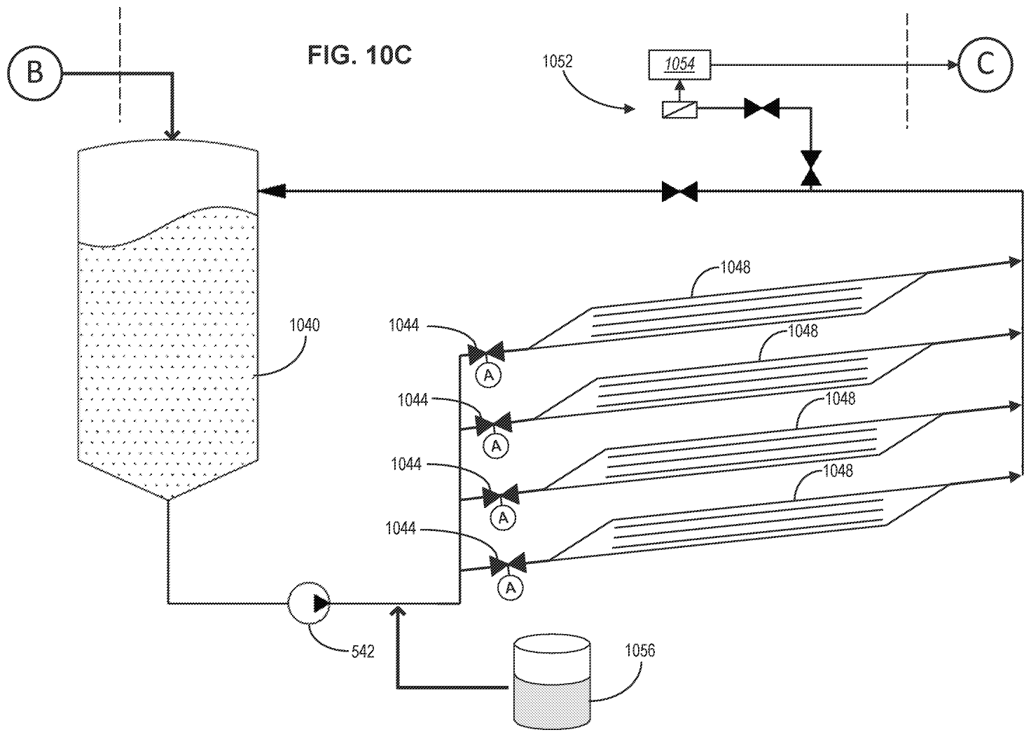

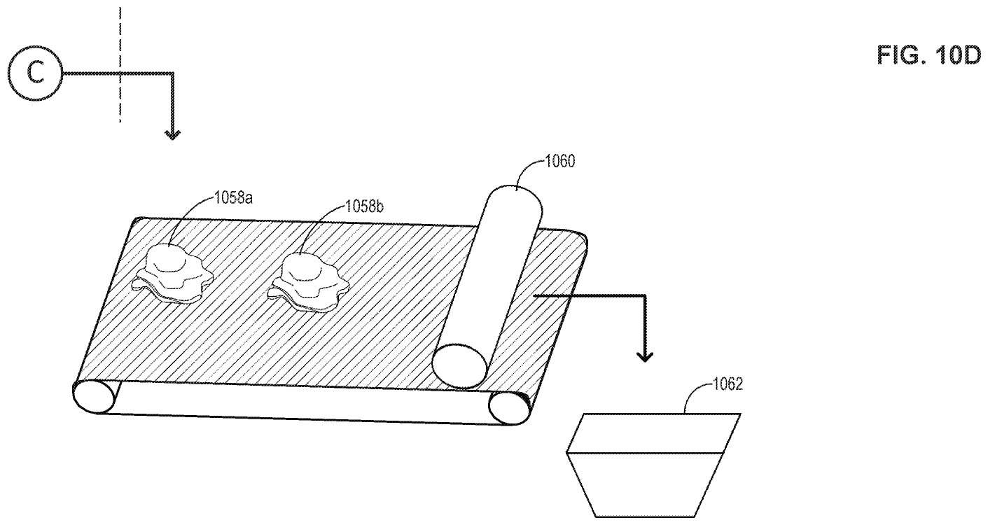

FIGS. 10A-10D and the following accompanying paragraphs describe procurement of cells and growth of cells into a cell mass in accordance with one or more embodiments. Generally, FIGS. 10A-10D illustrate a process of collecting cells from an animal, growing cells in a favorable environment, banking successful cells, and collecting cells into a cell mass followed by de-wetting and/or other treatments.

As illustrated by step 1002 in FIG. 10A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 1004 is removed from an animal. In some examples, the tissue 1004 is removed from a living animal by taking a skin sample from the living animal. For instance, skin or muscle samples may be taken from a chicken, cow, fish, shellfish or another animal.

Cells may be extracted from the tissue 1004 that was removed from the animal. More specifically, the tissue 1004 is broken down by enzymatic and/or mechanical means. To illustrate, FIG. 10A includes digested tissue 1006 that comprises the cells to be grown in cultivation.

Cells in the digested tissue 1006 may be proliferated under appropriate conditions to begin a primary culture. As illustrated in FIG. 10A, cells 1008 from the digested tissue 1006 are spread on a surface or substrate and proliferated until they reach confluence. As shown in FIG. 10A, in some cases, cells 1012 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below −80C. The protective agent may comprise dimethyl sulfoxide (DMSO) or glycerol.

Cells stored through cryopreservation may be used to replenish working cell stock. For instance, while a portion of the digested tissue 1006 is used as the cells 1008 spread on a surface or substrate, the remaining or excess digested tissue 1006 is transferred to cryovials 1010 for storage. Furthermore, the cells 1012 may be banked once reaching confluence and stored in cryovials 1014.

Once the cells 1012 have reached confluence, or just before the cells 1012 have reached confluence (e.g., occupation of about 80% of the substrate), the disclosed process comprises a series of cell passage steps. During cell passage, the cells 1012 are divided into one or more new culture vessels for continued proliferation. To illustrate, the cells 1012 may be diluted or spread on one or more surfaces or substrates to form the cells 1018. The cells 1018 are then grown 1016 to confluence, or just before confluence.

The cycle of dividing the cells 1012 into the cells 1018 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed method comprises immortalizing cells that have been grown and passaged for the determined number of cycles. For instance, the cells 1018 may be immortalized. As shown in FIG. 10B, cells 1020 have demonstrated a preferred growth capacity to proceed to immortalization. To achieve immortalization, the disclosed process transfects the cells 1020 with genes of interest. In one example telomerase reverse transcriptase (TERT) is introduced to the cells 1020. In some embodiments, the cells may be subjected to a selection process as known by those skilled in the art. The cells 1020 may then be passaged for a predetermined set of passaging cycles. In one example passaging cycle, the cells 1020 are grown to (or near) confluence 1024, then they are reseeded in new growth vessels, preserved in vials 1022, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 1026 may be stored in cryovials 1028 for cryopreservation and banking. Cells in suspension 1026 will begin to proliferate and the process begins a series of dilute and expand steps.

During dilution and expansion, cells are moved from growth vessels into newer, and progressively larger, growth vessels. For example, cells in suspension 1026 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 1030 and diluted with additional media. The dilute-and-expand steps are repeated using progressively larger vessels (e.g., the vessel 1031 and the vessel 1032) and/or progressive dilution until the cells reach a production-ready volume. For example, cells may be production ready at about a 1,000-100,000 liter scale at 5 million cells per mL. The cells may be banked in cryovials at any of the dilution and expansion cycles.

As part of preparing cells to form cell-based-meat products, the disclosed process comprises growing the cells as an adherent culture. Generally, cells that are grown attached to a substrate form a texture that more closely resembles tissue found in conventional meat. Thus, the cells may be transferred from growth in suspension to growth in an adherent reactor. For example, the cells grown in suspension in the vessel 1032 may be transferred to growth on a substrate. FIG. 10C illustrates a bioreactor system comprising a plurality of adherent bioreactors 1048 connecting in parallel to a media vessel 1040. The media vessel 1040 holds the cells grown in suspension media. In some implementations, cells from the vessel 1032 are transferred directly to a cell culture media (or just “media”) vessel 1040. In one example, the media vessel 1040 comprises the vessel 1032. The adherent bioreactors 1048 may comprise pipe-based bioreactors. As shown, a plurality of valves 1044 is secured to the plurality of adherent bioreactors 1048 to enable individual use and access of each of the adherent bioreactors 1048. For instance, to limit flow to only a first bioreactor of the plurality of adherent bioreactors 1048, the valve 1044 of the first bioreactor is opened while the remaining valves 1044 are closed. Furthermore, the bioreactor system can include a directional valve 1042 for changing between flow directions.

In some implementations, and as illustrated in FIG. 10C, cells (e.g., adherent cells or suspension adapted cells) are prepared by flowing cells suspended in media (e.g., cell culture media) across substrates in the plurality of adherent bioreactors 1048. More particularly, cells from the media vessel 1040 may contact or land on the substrates in the plurality of adherent bioreactors 1048. Cells and media that flowed through the adherent bioreactors 1048 are cycled back to the media vessel 1040. The media and cells can be cycled through the adherent bioreactors 1048 until a target adherent cell density is reached. For instance, in some implementations, the disclosed method comprises measuring a cell density of outflow from the adherent bioreactors 1048 to infer an adherent cell density.

The cells grow into adherent tissue within the adherent bioreactors 1048. Once they have grown to a target density, either according to a learned timing or according to a measured fluctuation in cell metabolism of components such as glucose and oxygen, then the adherent tissue is ready for removal. The removal process of the disclosed method uses a high-pressure flow to shear the adherent tissue off the substrate surfaces. In one example, wash buffer from a wash tank 1056 is flowed across the substrates in the adherent bioreactors 1048. The wash buffer and cell mixture are flowed through a filter 1052 where the cells are collected into one or more cell masses 1054.

The cell masses 1054 may be further processed to adjust moisture content. FIG. 10D illustrates an example apparatus for reducing moisture content in the cells. In particular, FIG. 10D illustrates a pressure apparatus 1060 that compresses the cell masses 1058a and 1058b. While FIG. 10D illustrates a mechanical method for adjusting moisture content of the cell masses 1058a and 1058b, other methods may be used to adjust moisture content. For example, the cell masses 1058a and 1058b may be mixed with a drying agent, vacuum dried, centrifuged, or otherwise dried. A moisture-adjusted-cell mass may be transferred to a container 1062 for additional processing. For example, the cell mass 1058a or 1058b may be removed from the container 1062 to be formed into a cell-based-meat product.

### 7. ADDITIONAL EMBODIMENTS

Embodiment 1. A method for increasing bioconversion efficiency of a myogenic cell, comprising:
(a) co-culturing a myogenic cell with a support cell, wherein the support cell line comprises a polynucleotide comprising a coding sequence of a gene of interest; and
(b) culturing the myogenic cell and the support cell in a cultivation infrastructure under conditions sufficient to induce formation of the myocytes and multinucleated myotubes from the myogenic cell.

Embodiment 2. A method for increasing cell density of a culture comprising a myogenic cell line, comprising:
(a) co-culturing a myogenic cell with a support cell, wherein the support cell line comprises a polynucleotide comprising a coding sequence of a gene of interest; and
(b) culturing the myogenic cell and the support cell in a cultivation infrastructure under conditions sufficient to induce proliferation of the myogenic cell.

Embodiment 3. A method for increasing myotube formation from a myogenic cell, comprising:
(a) co-culturing a myogenic cell with a support cell, wherein the support cell comprises a polynucleotide comprising a coding sequence of a gene of interest; and
(b) culturing the myogenic cell and the support cell in a cultivation infrastructure under conditions sufficient to induce myotube formation from the myogenic cell.

Embodiment 4. A method of producing a cell based meat product suitable for consumption, comprising:
(a) co-culturing a myogenic cell and a support cell, wherein the support cell comprises a polynucleotide comprising a coding sequence of a gene of interest;
(b) inducing myogenic specific differentiation, wherein the myogenic cells form myocytes and multinucleated myotubes;
(c) culturing the myocytes and multinucleated myotubes to generate skeletal muscle fibers; and
(d) isolating the skeletal muscle fibers and producing the cell based meat product suitable for consumption.

Embodiment 5. The method of any one of embodiments 1-4, wherein the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1 or 1:10 number of myogenic cells to number of support cells.

Embodiment 6. The method of any one of embodiments 1-5, wherein the myogenic cell is selected from: a myoblast, a myocyte, a satellite cell, a side population cell, a myogenic pericyte, a mesangioblast, a multinucleated myotube, a skeletal muscle fiber, or a combination thereof.

Embodiment 7. The method of any one of embodiments 1-5, wherein the myogenic cells are natively myogenic.

Embodiment 8. The method of any one of embodiments 1-5, wherein the myogenic cells are non-natively myogenic.

Embodiment 9. The method of any one of embodiments 1-8, wherein the support cell is selected from: a fibroblast, a myofibroblast, a mesenchymal cell, an epithelial cell, and a stromal cell.

Embodiment 10. The method of any one of embodiments 1-9, wherein the gene of interest is selected from: FAP, IGF2, SDC4, SPHK1, and FAK, or a combination thereof.

Embodiment 11. The method of embodiment 10, wherein the gene of interest is FAP.

Embodiment 12. The method of embodiment 11, wherein FAP comprises an amino acid sequence having at least 80% sequence identity to a sequence selected from SEQ ID NOs: 1-11.

Embodiment 13. The method of embodiment 12, wherein FAP comprises an amino acid sequence selected from SEQ ID NO: 1-11.

Embodiment 14. The method of embodiment 10, wherein the gene of interest is IGF2.

Embodiment 15. The method of embodiment 14, wherein IGF2 comprises an amino acid sequence having at least 80% sequence identity to a sequence selected from SEQ ID NOs: 12-44.

Embodiment 16. The method of embodiment 15, wherein IGF2 comprises an amino acid sequence selected from SEQ ID NO: 12-44.

Embodiment 17. The method of embodiment 10, wherein the gene of interest is SDC4.

Embodiment 18. The method of embodiment 17, wherein SDC4 comprises an amino acid sequence having at least 80% sequence identity to a sequence selected from SEQ ID NOs: 45-57.

Embodiment 19. The method of embodiment 18, wherein SDC4 comprises an amino acid sequence selected from SEQ ID NO: 45-57.

Embodiment 20. The method of embodiment 10, wherein the gene of interest is SPHK1.

Embodiment 21. The method of embodiment 20, wherein SPHK1 comprises an amino acid sequence having at least 80% sequence identity to a sequence selected from SEQ ID NOs: 58-83.

Embodiment 22. The method of embodiment 21, wherein SPHK1 comprises an amino acid sequence selected from SEQ ID NO: 58-83.

Embodiment 23. The method of embodiment 10, wherein the gene of interest is FAK.

Embodiment 24. The method of embodiment 23, wherein FAK comprises amino acid sequence having at least 80% sequence identity to a sequence selected from SEQ ID NOs: 84-96.

Embodiment 25. The method of embodiment 24, wherein FAK comprises an amino acid sequence selected from SEQ ID NO: 84-96.

Embodiment 26. The method of any one of embodiments 1-25, wherein the myogenic cell comprises a polynucleotide comprising a coding sequence of a gene of interest.

Embodiment 27. The method of embodiment 26, wherein the gene of interest is IGF2 or genetic variant thereof.

Embodiment 28. The method of any one of embodiments 1-27, wherein the myogenic cell comprises a polynucleotide comprising a coding sequence a myogenic transcription factor.

Embodiment 29. The method of embodiment 28, wherein the myogenic transcription factor is selected from MYOD1, MYOG, MYF5, MYF6, PAX3, PAX7, or genetic variants thereof.

Embodiment 30. The method of any one of embodiments 1-29, wherein co-culturing a myogenic cell with a support cell comprising a polynucleotide comprising a coding sequence of a gene of interest results in increased myotube formation, myogenin expression, myosin heavy chain expression, or a combination thereof as compared to a myogenic cell not exposed to the methods of any one of embodiments 1-29.

Embodiment 31. The method of any one of embodiments 1-30, wherein a myogenic cell co-cultured with a support cell comprising a polynucleotide comprising a coding sequence of a gene of interest comprises higher total protein as compared to a myogenic cell not cultured with the support cell comprising a polynucleotide comprising a coding sequence of a gene of interest.

Embodiment 32. The method of any one of embodiments 1-31, wherein a myogenic cell co-cultured with the support cell comprising a polynucleotide comprising a coding sequence of a gene of interest comprises a higher proliferation rates as compared to a myogenic cell not cultured with a support cell comprising a polynucleotide comprising a coding sequence of a gene of interest.

Embodiment 33. The method of any one of embodiments 1-32, wherein the myogenic cells, the support cells, or both, are immortalized.

Embodiment 34. The method of any one of embodiments 1-32, further comprising an immortalizing step, wherein the myogenic cells, the support cells, or both are immortalized.

Embodiment 35. The method of embodiment 33 or 34, wherein the immortalization is selected from a method comprising: transducing with a polynucleotide encoding TERT, transducing with a polynucleotide encoding CDK4/6, transducing with a polynucleotide Cyclin D1, inactivating a gene encoding an inhibitor of cyclin-dependent kinase 4/6 (CDK4/6), inactivating a gene encoding an inhibitor of Cyclin D1, or a combination thereof.

Embodiment 36. The method of any one of embodiments 1-35, wherein the co-culturing, culturing steps, or both, comprises contacting the myogenic cell, support cell, or both with a growth medium.

Embodiment 37. The method of embodiment 36, wherein the growth media comprises DMEM/F12, fetal bovine serum, chicken serum and fibroblast growth factor 2.

Embodiment 38. The method of embodiment 36 or 37, wherein the growth media further comprises a TGF-beta inhibitor, an activin A inhibitor, and a WNT activator.

Embodiment 39. The method of any one of embodiments 1-38, wherein the co-culturing and/or culturing steps comprises contacting the myogenic cell, support cell, or both with a differentiation medium.

Embodiment 40. The method of embodiment 39, wherein the differentiation medium bovine serum, chicken serum, horse serum, or a combination thereof.

Embodiment 41. The method of any one of embodiments 1-40, wherein the myogenic cells are from a livestock, poultry, game, or aquatic animal species.

Embodiment 42. The method of any one of embodiments 1-41, wherein the myogenic cells are from a chicken, a duck, or turkey.

Embodiment 43. The method of any one of embodiments 1-41, wherein the myogenic cells are from a fish.

Embodiment 44. The method of any one of embodiments 1-41, wherein the myogenic cells are from a livestock species.

Embodiment 45. The method of embodiment 44, wherein the livestock species is porcine or bovine.

Embodiment 46. The method of any one of embodiments 1-40, wherein the myogenic cells are from any animal species intended for human or non-human dietary consumption.

Embodiment 47. The method of any one of embodiments 1-46, wherein the support cells are from a livestock, poultry, game, or aquatic animal species.

Embodiment 48. The method of any one of embodiments 1-47, wherein the support cells are from a chicken, a duck, or turkey.

Embodiment 49. The method of any one of embodiments 1-47, wherein the support cells are from a fish.

Embodiment 50. The method of any one of embodiments 1-47, wherein the support cells are from a livestock species.

Embodiment 51. The method of embodiment 50, wherein the livestock species is porcine or bovine.

Embodiment 52. The method of any one of embodiments 1-46, wherein the support cells are from any animal species intended for human or non-human dietary consumption.

Embodiment 53. The method of any one of embodiments 1-3 and 5-51, further comprising inducing myogenic specific differentiation, wherein the differentiated cells form myocytes and multinucleated myotubes.

Embodiment 54. The method of embodiment 53, wherein the myocytes and multinucleated myotubes form a skeletal muscle fiber.

Embodiment 55. The method of embodiment 54, further comprising isolating the skeletal muscle fiber and producing the cell based meat product suitable for consumption.

Embodiment 56. A myogenic cell produced using the methods of any one of embodiments 1-55.

Embodiment 57. A cell based meat product suitable for consumption produced using the methods of any one of embodiments 1-55.

Embodiment 58. A cell-based meat product suitable for consumption, comprising: (a) a myogenic cell; and (b) a support cell comprising a polynucleotide comprising a coding sequence of a gene of interest.

Embodiment 59. The cell-based meat product of embodiment 58, wherein the myogenic cell and the support cell are co-cultured at a ratio of 1:1, 1:2, 2:1, 1:3: 3:1, 1:4, 4:1, 1:5, 5:1, 1:6, 6:1, 1:7, 7:1, 1:8, 8:1, 1:9, 9:1, 10:1 or 1:10 number of myogenic cells to number of support cells.

Embodiment 60. The cell-based meat product of embodiment 58 or 59, wherein the myogenic cell is selected from: a myoblast, a myocyte, a satellite cell, a side population cell, a muscle derived stem cell, a myogenic pericyte, a mesangioblast, a multinucleated myotube, a skeletal muscle fiber, or a combination thereof.

Embodiment 61. The cell-based meat product of any one of embodiments 58-60, wherein the myogenic cells are natively myogenic.

Embodiment 62. The cell-based meat product of any one of embodiments 58-60, wherein the myogenic cells are non-natively myogenic.

Embodiment 63. The cell-based meat product of any one of embodiments 58-62, wherein the support cell is selected from: a fibroblast, a myofibroblast, a mesenchymal cell, an epithelial cell, and a stromal cell.

Embodiment 64. The cell-based meat product of any one of embodiments 58-63, wherein the gene of interest is selected from: FAP, IGF2, SDC4, SPHK1, and FAK, or a combination thereof.

Embodiment 65. The cell-based meat product of embodiment 64, wherein the gene of interest is FAP.

Embodiment 66. The cell-based meat product of embodiment 65, wherein FAP comprises an amino acid sequence having at least 80% sequence identity to a sequence selected from SEQ ID NOs: 1-11.

Embodiment 67. The cell-based meat product of embodiment 66, wherein FAP comprises an amino acid sequence selected from SEQ ID NO: 1-11.

Embodiment 68. The cell-based meat product of embodiment 64, wherein the gene of interest is IGF2.

Embodiment 69. The cell-based meat product of embodiment 68, wherein IGF2 comprises an amino acid sequence having at least 80% sequence identity to a sequence selected from SEQ ID NOs: 12-44.

Embodiment 70. The cell-based meat product of embodiment 69, wherein IGF2 comprises an amino acid sequence selected from SEQ ID NO: 12-44.

Embodiment 71. The cell-based meat product of embodiment 64, wherein the gene of interest is SDC4.

Embodiment 72. The cell-based meat product of embodiment 71, wherein SDC4 comprises an amino acid sequence having at least 80% sequence identity to a sequence selected from SEQ ID NOs: 45-57.

Embodiment 73. The cell-based meat product of embodiment 72, wherein SDC4 comprises an amino acid sequence selected from SEQ ID NO: 45-57.

Embodiment 74. The cell-based meat product of embodiment 64, wherein the gene of interest is SPHK1.

Embodiment 75. The cell-based meat product of embodiment 74, wherein SPHK1 comprises an amino acid sequence having at least 80% sequence identity to a sequence selected from SEQ ID NOs: 58-83.

Embodiment 76. The cell-based meat product of embodiment 75, wherein SPHK1 comprises an amino acid sequence selected from SEQ ID NO: 58-83.

Embodiment 77. The cell-based meat product of embodiment 64, wherein the gene of interest is FAK.

Embodiment 78. The cell-based meat product of embodiment 77, wherein FAK comprises amino acid sequence having at least 80% sequence identity to a sequence selected from SEQ ID NOs: 84-96.

Embodiment 79. The cell-based meat product of embodiment 78, wherein FAK comprises an amino acid sequence selected from SEQ ID NO: 84-96.

Embodiment 80. The cell-based meat product of any one of embodiments 58-79, wherein the myogenic cell comprises a polynucleotide comprising a coding sequence of a gene of interest.

Embodiment 81. The cell-based meat product of embodiment 80, wherein the gene of interest is IGF2 or genetic variant thereof.

Embodiment 82. The cell-based meat product of any one of embodiments 58-81, wherein the myogenic cell comprises a polynucleotide comprising a coding sequence a myogenic transcription factor.

Embodiment 83. The cell-based meat product of embodiment 82, wherein the myogenic transcription factor is selected from MYOD1, MYOG, MYF5, MYF6, PAX3, PAX7, or genetic variants thereof.

Embodiment 84. The cell-based meat product of any one of embodiments 58-83, wherein the myogenic cells are from a livestock, poultry, game, or aquatic animal species.

Embodiment 85. The cell-based meat product of any one of embodiments 58-83, wherein the myogenic cells are from a chicken, a duck, or turkey.

Embodiment 86. The cell-based meat product of any one of embodiments 58-83, wherein the myogenic cells are from a fish.

Embodiment 87. The cell-based meat product of any one of embodiments 58-83, wherein the myogenic cells are from a livestock species.

Embodiment 88. The cell-based meat product of embodiment 87, wherein the livestock species is porcine or bovine.

Embodiment 89. The cell-based meat product of any one of embodiments 58-84, wherein the myogenic cells are from any animal species intended for human or non-human dietary consumption.

Embodiment 90. The cell-based meat product of any one of embodiments 58-89, wherein the support cells are from a livestock, poultry, game, or aquatic animal species.

Embodiment 91. The cell-based meat product of any one of embodiments 58-90, wherein the support cells are from a chicken, a duck, or turkey.

Embodiment 92. The cell-based meat product of any one of embodiments 58-90, wherein the support cells are from a fish.

Embodiment 93. The cell-based meat product of any one of embodiments 58-90, wherein the support cells are from a livestock species.

Embodiment 94. The cell-based meat product of embodiment 93, wherein the livestock species is porcine or bovine.

Embodiment 95. The cell-based meat product of any one of embodiments 58-94, wherein the support cells are from any animal species intended for human or non-human dietary consumption.

Embodiment 96. The cell-based meat product of any one of embodiments 58-95, wherein the cell-based meat product suitable for consumption is a raw, uncooked food product.

Embodiment 97. The cell-based meat product of any one of embodiments 58-96, wherein the cell-based meat product suitable for consumption is a cooked food product.

### 8. EXAMPLES

### 8.1 Summary of Experimental Observations

Applicant evaluated how co-culturing a myogenic cell with a support cell impacted the myogenic cell's ability to form myotubes. In particular, Applicant evaluated how support cells (e.g., chicken fibroblast 1312 cells) engineered to express different genes of interest: FAK, FAP, IGF2, SDC4, or SPHK1 impacted the myogenic cell's ability to form myotubes. Applicant found that co-culturing myogenic cells with fibroblasts engineered to express FAK, FAP, IGF2, SDC4, or SPHK1 improved myotube formation as compared to either the no co-culture control or co-culture with fibroblasts not engineered to overexpress one of the genes of interest.

Applicant also evaluated whether engineering the myogenic cell prior to co-culturing would impact myotube formation. Applicant found that engineering myogenic cells to express IGF2 prior to co-culturing myogenic cells with fibroblasts engineered to express FAK, FAP, IGF2, SDC4, or SPHK1 improved myotube formation as compared controls (i.e., no co-culture control or co-culture with fibroblasts not engineered to overexpress one of the genes of interest) in most of the conditions tested.

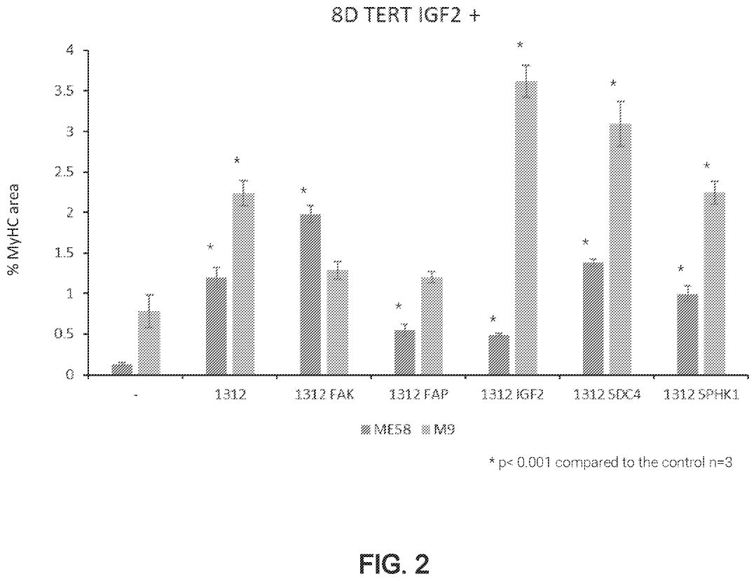

Lastly, Applicant also evaluated whether co-culturing (i.e., a myogenic cell and an engineered support cell) in medium containing a small molecule cocktail would impact myotube formation. In these experiments, Applicant evaluated myogenic cells engineered to express IGF2 and myogenic cells not engineered to express IGF2 in combination with co-culturing the myogenic cells with support cells engineered to express FAK, FAP, IGF2, SDC4, or SPHK1. Applicant found that in conditions where the myogenic cells were not engineered to express IGF2 but co-cultured with engineered support cells the small molecule cocktail increased myotube formation in a subset of conditions. Interestingly, for myogenic cells engineered to express IGF2 and co-cultured with engineered support cells in medium comprising the small molecule cocktail, Applicant found synergistic effects of the engineered myogenic cells and small molecule cocktails (See FIG. 2).

Overall, this work demonstrated the ability to enhance myotube formation and subsequent generation of cell based meat products suitable for consumption by co-culturing myogenic cells with support cells engineered to express a gene of interest. These findings are important because manufacturing cells for cell based meat products can include tailoring the methods so that the cells that make up the cell based meat products have appropriate texture profiles. In such cases, these texture profiles can depend, at least in part, on the cell's ability to form myotubes and/or contain muscle proteins such as MyHC. The engineered support cells provided herein supply sufficient paracrine and juxtacrine signaling to the myogenic cells-thereby facilitating growth and differentiation of the myogenic cells with the desired properties (i.e., texture profiles). The methods described herein provide a means for increasing the efficiency (i.e., the bioconversion efficiency) with which myogenic cells form myotubes expressing MyHC, thereby increasing the efficiency with which cell based meat products that include the desired texture profiles can be produced.

### 8.2 Experimental Procedures/Methods

### Cell Line Production

In order to generate cells lines with integrated polynucleotides, a PhiC31 Integrase Expression Plasmid system was used (System Biosciences, Cat No. FC200PA-1). Briefly, a coding sequence of a gene of interest was cloned into a PhiC31 integrase expression plasmid. Fibroblast cell lines (e.g., fibroblasts) were transfected with the plasmid containing the coding sequences of the gene(s) of interest and a plasmid containing an integrase (PhiC31) to integrate the coding sequences into the genome of the cell line. Fibroblast cell lines or myogenic cell lines with integrated plasmids were selected using neomycin and assessed for transgene expression with QPCR. Cell lines exhibiting stable expression were selected for further analysis and co-culture experiments.

Chicken 1312fibroblasts. cDNA encoding genes of interest (i.e., FAK, FAP, IGF2, SDC4, and/or SPHK1) were cloned into a PhiC31 integrase vector. Chicken fibroblast cells (1312 cells) were transduced with the PhiC31 vector containing a gene of interest along with an integrase. Following transduction, 1312 cells were exposed to media comprising a puromycin to select cells stably expressing the gene of interest.

8D Myoblasts. Chicken 8D myoblasts were immortalized using TERT (according to the methods described herein and as described in WO2017124100A1, which is herein incorporated by reference in its entirety).

8D Myoblasts+IGF2. Chicken 8D myoblasts immortalized (e.g., according to the methods described herein (e.g., WO2017124100A1, which is herein incorporated by reference in its entirety)) was transduced with an additional polynucleotide comprising a coding sequence of an IGF2 protein. 8D myoblasts expressing TERT were exposed to media comprising neomycin to select cells stably expressing both TERT and IGF2.

### Assessment of Myogenicity

Using qRT-PCR (real-time quantitative reverse transcription). Messenger RNA (mRNA) is isolated from cells to examine gene expression with probes specifically designed to amplify select target genes to characterize cell lines. Identical quantity of mRNA is reverse transcribed to generate cDNA. Each cDNA is submitted to quantitative PCR (qPCR) to assess the expression of myogenic factors relative to a housekeeping gene. Expression of MyoD, MyoG, and/or MyHC1e indicate myogenic cells. Additionally, high levels of MyHC1e are indicative of cells that can mature to form myotubes.

Using immunohistochemistry. Myogenic cells were seeded into a collagen/fibronectin coated 96-well plate at a density (about 5000-10,000 cells/cm²). For co-cultures, myogenic cells and fibroblasts were seeded 1:1 into a collagen/fibronectin coated 96-well plate at a density (about 5000-10,000 cells/cm² per cell type). After 2 days of culture, cells are fixed with 4% paraformaldehyde (PFA) and washed. Cells were permeabilized with 0.05% PBS-T (triton-x), blocked with normal goat serum (Thermo Fisher) and are incubated with 1:150 of an anti-MyHC antibody and 1:1000 DAPI and imaged.

### 8.3 Example 1: Improving Myotube Formation Using Co-Culture of Myogenic Cells with Fibroblasts Engineered to Express a Gene of Interest

This experiment was designed to evaluate how co-culturing myoblasts with fibroblasts that express particular genes of interest impacted myotube formation as assessed by MyHC staining.

For these experiments, Chicken 1312 fibroblasts expressing genes of interest (generated as described in Section 5.1) and 8D TERT (+/−IGF-2) (generated as described in Section 5.1) were seeded at 1:1 ratio at a final density of 10,000 cells/cm² per cell type in a 96-well plate into a collagen/fibronectin coated 96-well plate. Once the cells reached confluence, the media was replaced with DMEM/F12 containing 2% horse serum. After 3 days, the plate was fixed and stained with 1:150 MyHC as a readout for myotube formation.

In addition to testing how co-culture impacted myotube formation, cells were also incubated with proliferation media ME58 with and without small molecule cocktail (ME9: DMEM/F12, about 20% FBS, about 5% chicken serum, CHIR99021, A-83-01, and LDN193189) for 2-3 days. When the cells reached confluence, the media was changed for differentiation media (2% horse serum). After 3 days, the cells were fixed and stained for MyHC as a readout for myotube formation.

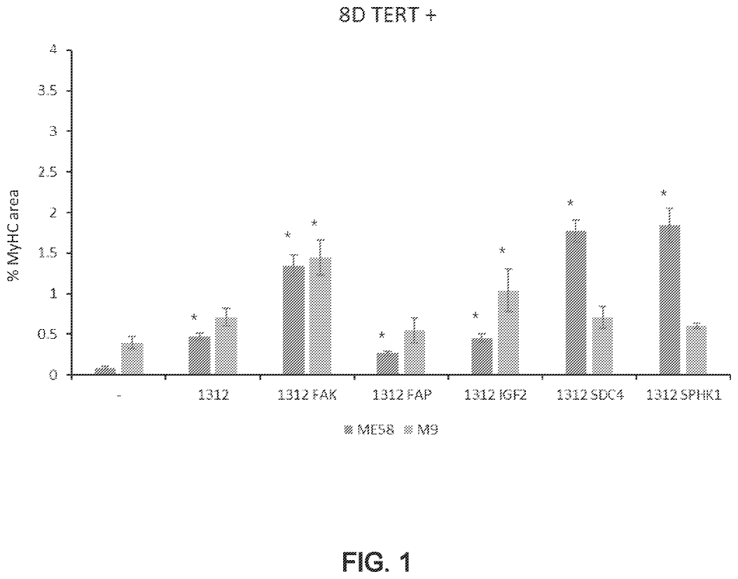

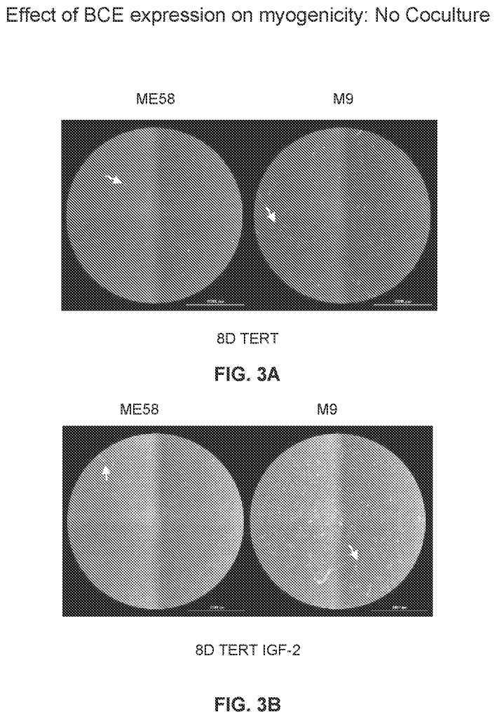

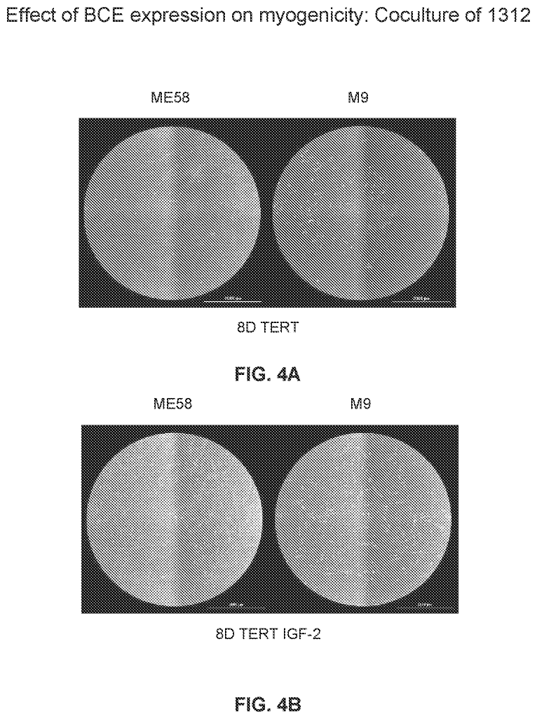

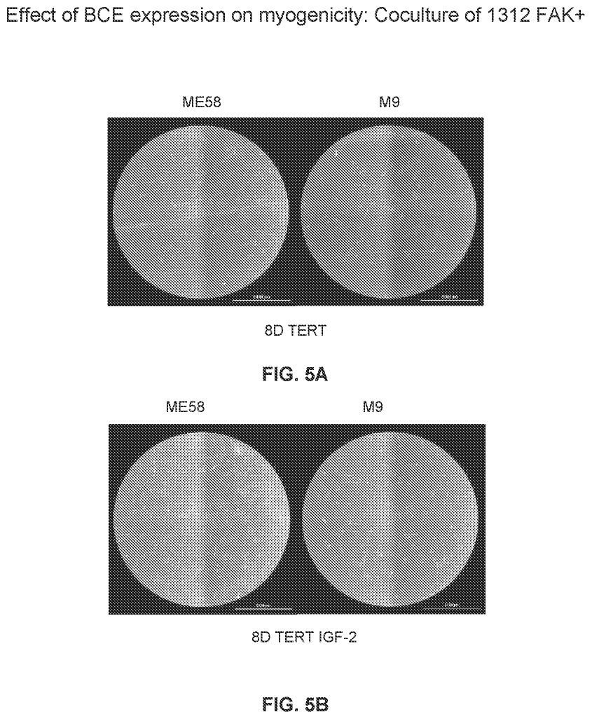

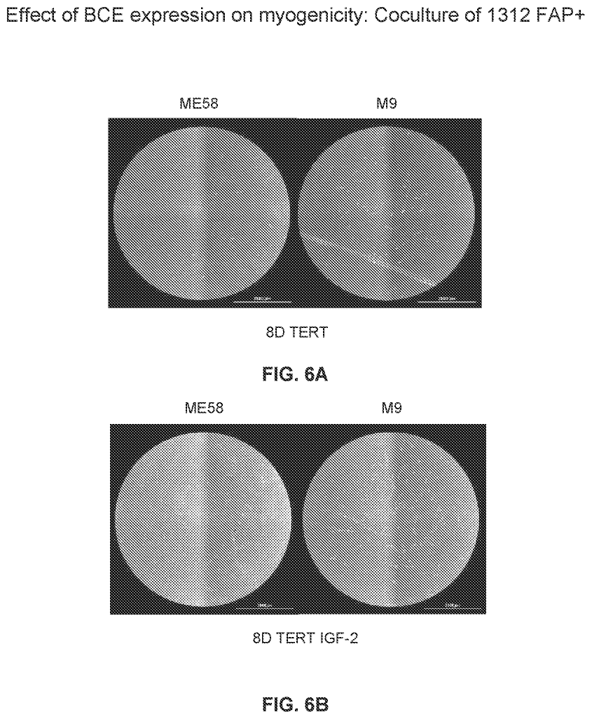

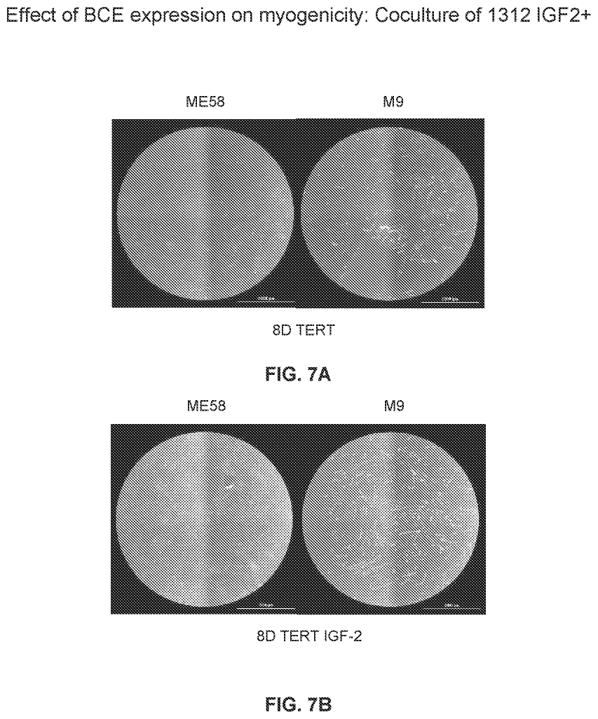

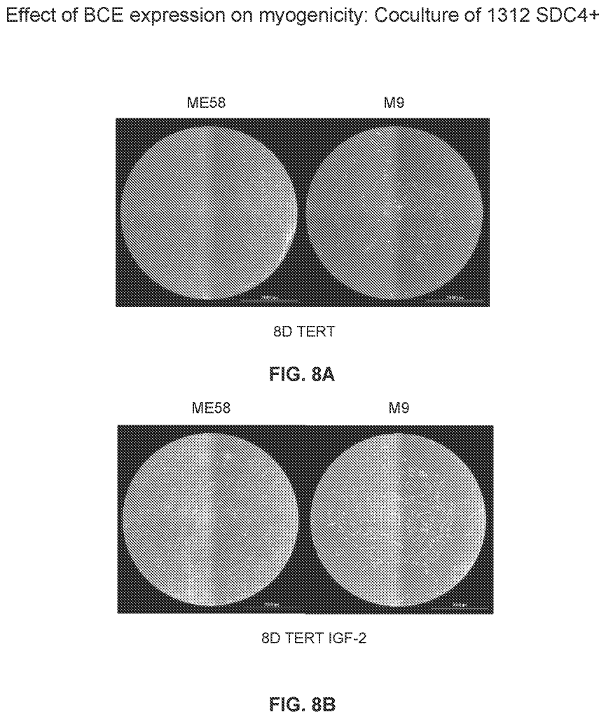

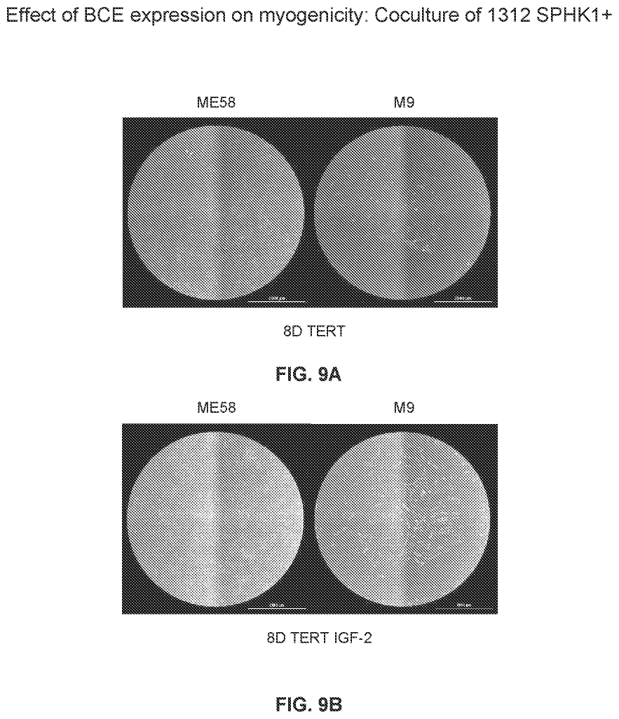

As shown in FIG. 1, which quantifies the images shown in FIGS. 3A, 4A, 5A, 6A, 7A, 8A, and 9A, co-culture of 8D TERT myoblasts with fibroblasts overexpressing one of the genes of interest (i.e., genes listed on the x-axis) in the presence of ME58 increased myotube formation (as measured by % MyHC area) for FAK, SDC4, and SPHK1 as compared to both controls (no co-culture control (indicated as “−” in FIG. 1) and co-cultured fibroblasts not containing polynucleotide containing a gene of interest (indicated as “1312” in FIG. 1). Co-culturing the 8D TERT myoblasts with fibroblasts overexpression FAP and IGF2 increase myotube formation as compared to the no co-culture control only. FIG. 1 also shows that co-culture of 8D TERT myoblasts with fibroblasts overexpressing one of the genes of interest (i.e., gene listed on the x-axis) in the presence of ME9 increased myotube formation for FAK and IGF2 as compared to both controls and FAP, IGF2, SDC4 and SPHK1 as compared to the no co-culture control. Statistical significance was calculated for each condition as compared to the no co-culture control (as indicated by (−) in FIG. 1). Statistically significant differences between the means were determined by an ANOVA one-way test with P-values indicated with asterisks. N=4. * p<0.001.

In summary, FIG. 1 shows that co-culturing 8D TERT myoblasts with fibroblasts overexpressing one of the genes of interest in either ME58 or ME58+ME9 media both resulted in statistically significant increases in myotube formation (measured by % MyHC area) as compared to the controls.

As shown in FIG. 2, which quantifies the images shown in FIGS. 3B, 4B, 5B, 6B, 7B, 8B, and 9B, co-culturing 8D TERT+IGF2 myoblasts with fibroblasts overexpressing one of the genes of interest (i.e., genes listed on the x-axis) in the presence of ME58 increased myotube formation (as measured by % MyHC area) for FAK and SDC4 as compared to both controls. Co-cultures including fibroblasts overexpressing FAP IGF2, and SPHK1 showed increased myotube formation (as measured by % MyHC area) as compared to the no co-culture control. FIG. 2 also shows that co-culture of 8D TERT+IGF2 myoblasts with fibroblasts over expressing one of the genes of interest (i.e., genes listed on the x-axis) in the presence of ME58+ME9 increased myotube formation for IGF2, SDC4, and SPHK1 as compared to both controls and for FAK and FAP as compared to the no co-culture control. Statistical significance was calculated for each condition as compared to the no co-culture control (as indicated by (“−”) in FIG. 2. Statistically significant differences between the means were determined by an ANOVA one-way test with P-values indicated with asterisks. N=3. * p<0.001.

In summary, FIG. 2 shows that co-culturing 8D TERT+IGF2 myoblasts with fibroblasts in ME58+ME9 media had a synergistic impact on myotube formation (as measured by % MyHC area). This suggests that by first introducing a polynucleotide comprising a coding sequence for IGF2 into an 8D TERT myoblast enhances myotube formation when co-cultured with fibroblasts expressing IGF2, SDC4, and SPHK1 in ME58+ME9 culture media.

### 8.4 Example 2: Assessment of Panel of Engineered Cell Lines Show Engineered Cells Significantly Improved Cell Density

This experiment is designed to assess impact of co-culturing myogenic cells with engineered support cells on cell density. Without wishing to be bound by theory, the engineered support cells enable increased proliferation of the myogenic cells by signaling through the integrin and/or mTOR signaling pathways.

In these experiments, chicken myoblasts are co-cultured with support cells engineered to express one or more of the genes of interest (e.g., FAK, FAP, IGF2, SDC4, and SPHK1) and cultured according to the methods described in Example 1. Following co-culture cell density is measured by determining viable cell density.

### 8.5 Example 3: Methods for Producing Cells Suitable for Consumption

The manufacturing of cultured muscle cells suitable for consumption, in one exemplary protocol, can comprise: culturing a population of myoblasts in a first suspension culture and culturing a population of fibroblast engineered to include a polynucleotide comprising a coding sequence of a gene of interest (e.g., FAK, FAP, IGF2, SDC4, and SPHK1) in a second suspension culture. The myoblasts and fibroblasts are combined and cultured at a ratio of 1:1 as an adherent co-culture in a cultivation infrastructure (e.g., a bioreactor). After the co-cultured cells have proliferated to confluence, the culture medium is removed, and the adherent cell cultures are rinsed with phosphate buffered saline. Next, the confluent biomass of adherent cells are mechanically, fluidically, enzymatically, or metabolically dissociated from the substrate by means of a scraping device, a pressurized fluid, or a harvest media. The dissociated biomass is collected into centrifuge tubes, pelleted to remove excess liquid, and processed for food product preparation.

### 9. EQUIVALENTS AND INCORPORATION BY REFERENCE

All references cited herein are incorporated by reference to the same extent as if each individual publication, database entry (e.g., Genbank sequences, GeneID entries, or Entrez Gene), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

10 SEQUENCE APPENDIX

| SEQ | | |
|---|---|---|
| ID NO: | Description | Sequence |
| 1 | NP_001091470.1 | MKTWLKIVFGVATSAVLALLVMCIVLRPSRVHNSEESTTRALTLKDILNGT |
| | FAP | FSYKTFFPNWISGQEYLHQSTDNNVVFYNIETGESYTILSNTTMKSVNASN |
| | [organism = Bos | YGLSPDRQFAYLESDYSKLWRYSYTATYHIYDLINGEFIRRNELPRPIQYLC |
| | taurus] | WSPVGSKLAYVYQNNIYLKQRPEDPPFQITYNGKENKIFNGIPDWVYEEEM |
| | [GeneID = 508882] | LATKYALWWSPNGKFLAYAEFNDTEIPVIAYSYYGDEQYPRTINIPYPKAG |
| | | AKNPVVRIFIIDATYPEHIGPREVPVPAMIASSDYYFSWLTWVTDDRICLQW |
| | | LKRIQNVSVLSTCDFREDWQTWNCPKTQEHIEESRTGWAGGFFVSTPVFSH |
| | | DTISYYKIFSDKDGYKHIHYIRDTVENAIQITSGKWEAINIFRVTQDSLFYSS |
| | | NEFEGYPGRRNIYRISIGSHSPSKKCITCHLRKKRCQYYTASFSDYAKYYAL |
| | | VCYGPGLPISTLHDGRTDQEIKILEDNKELENALKNIQLPKEEIKKLKVDDIT |
| | | LWYKMILPPQFDKSKKYPLLIQVYGGPCSQSVRSIFAVSWISYLASKEGIVI |
| | | ALVDGRGTAFQGDKLLYAVYRKLGVYEVEDQITAVRKFIEMGFIDEKRIAI |
| | | WGWSYGGYVSSLALASGTGLFKCGIAVAPVSSWEYYASIYTERFMGLPTK |
| | | DDNLKHYKNSTVMARAEYFRNVDYLLIHGTADDNVHFQNSAQIAKALVN |
| | | AQVDFQAMWYSDQNHGLSGLSTKHLYTHMTHFLKQCFSLSD |
| 2 | >XP_010800270.1 | MKTWLKIVFGVATSAVLALLVMCIVLRPSRVHNSEESTTRALTLKDILNGT |
| | FAP | FSYKTFFPNWISGQEYLHQSTDNNVVFYNIETGESYTILSNTTMKSVNASN |
| | [organism = Bos | YGLSPDRQFAYLESDYSKLWRYSYTATYHIYDLTNGEFIRRNELPRPIQYLC |
| | taurus] | WSPVGSKLAYVYQNNIYLKQRPEDPPFQITYNGKENKIFNGIPDWVYEEEM |
| | [GeneID = 508882] | LATKYALWWSPNGKFLAYAEFNDTEIPVIAYSYYGDEQYPRTINIPYPKAG |
| | [isoform = X1] | AKNPVVRIFIIDATYPEHIGPREVPVPAMIASSDYYFSWLTWVTDDRICLQW |
| | | LKRIQNVSVLSTCDFREDWQTWNCPKTQEHIEESRTGWAGGFFVSTPVFSH |
| | | DTISYYKIFSDKDGYKHIHYIRDTVENAIQITSGKWEAINIFRVTQDSLFYSS |
| | | NEFEGYPGRRNIYRISIGSHSPSKKCITCHLRKKRCQYYTASFSDYAKYYAL |
| | | VCYGPGLPISTLHDGRTDQEIKILEDNKELENALKNIQLPKEEIKKLKVDDIT |
| | | LWYKMILPPQFDKSKKYPLLIQVYGGPCSQSVRSIFAVSWISYLASKEGIVI |
| | | ALVDGRGTAFQGDKLLYAVYRKLGVYEVEDQITAVRKFIEMGFIDEKRIAI |
| | | WGWSYGGYVSSLALASGTGLFKCGIAVAPVSSWEYYGAPVSTIFSCISSPG |
| | | LLRQLAEQMTRVEHHRKESKTFPQQLPLGD |
| 3 | >XP_010800271.1 | MKTWLKIVFGVATSAVLALLVMCIVLRPSRVHNSEESTTRALTLKDILNGT |
| | FAP | FSYKTFFPNWISGQEYLHQSTDNNVVFYNIETGESYTILSNTTMKSVNASN |
| | [organism = Bos | YGLSPDRQFAYLESDYSKLWRYSYTATYHIYDLINGEFIRRNELPRPIQYLC |
| | taurus] | WSPVGSKLAYVYQNNIYLKQRPEDPPFQITYNGKENKIFNGIPDWVYEEEM |
| | [GeneID = 508882] | LATKYALWWSPNGKFLAYAEFNDTEIPVIAYSYYGDEQYPRTINIPYPKAG |
| | [isoform = X2] | AKNPVVRIFIIDATYPEHIGPREVPVPAMIASSDYYFSWLTWVTDDRICLQW |
| | | LKRIQNVSVLSTCDFREDWQTWNCPKTQEHIEESRTGWAGGFFVSTPVFSH |
| | | DTISYYKIFSDKDGYKHIHYIRDTVENAIQITSGKWEAINIFRVTQDSLFYSS |
| | | NEFEGYPGRRNIYRISIGSHSPSKKCITCHLRKKRCQYYTASFSDYAKYYAL |
| | | VCYGPGLPISTLHDGRTDQEIKILEDNKELENALKNIQLPKEEIKKLKVDDIT |
| | | LWYKMILPPQFDKSKKYPLLIQVYGGPCSQSVRSIFAVSWISYLASKEGIVI |
| | | ALVDGRGTAFQGDKLLYAVYRKLGVYEVEDQITAVSPMEVMFHRWPLLQ |
| | | ELVFSNVG |
| 4 | >NP_001180568.2 | MKTRLKVLFGVVVCVLLSLLLMCIVLLPSKVVTTVDGPRALTLDDYLNGN |
| | FAP | FQYKTYFPYWVSDSEYLHQNQEDDIILFNVDMNYLTTIMTNSTMKQVNAS |
| | [organism = Gallus | NYVMSSDKYFIALESNYSKLWRYSYTASYHIYDLIYGGFVTENQLPHKIQY |
| | gallus] | ISWSPVGHKLAYVYQNNIYLKQSPREAPIKLTSDGKENEIFNGIPDWVYEEE |
| | [GeneID = 424186] | MLATKYALWWSPSGKYLAYVQFNDSDIPVIEYSYFGEDQYPRKIIIPYPKA |
| | | GAKNPTVKVFIVDTTNIEAFGPKEVPVPAVIASSDHYFTWLTWVTDSRVGV |
| | | QWLKRIQNFSVLAICDFKENSNTWDCPENQQHIEESQTGWAGGFFVSAPYF |
| | | TSDGSSYYKIFSDKNGYKHIHYINGSVENAIQITSGEWEAIYIFRVINDAIFY |
| | | SSNEFEGYPGRRNIYKISIGSKPIRKLCITCNLRKERCQYYTARFSERSKYYA |
| | | LICYGPGIPISTLFENESDRELRILEDNQELQSALQEIILPKEEINKLEVDGITL |
| | | WYKMLIPPQFDRSKKYPLLIQVYGGPCSQNVKHTFSISWITYLASKEGIIVA |
| | | LVDGRGTAYQGDKILHAVYRRLGVYEVEDQISAVKKFIEMGFIDEKRIAIW |
| | | GWSYGGYVTSLALGSGSGVFKCGIAVAPVSSWEYYASIYTERFMGLPVES |
| | | DNLEHYKNSTVMARAKNFQNVEYLLIHGTADDNVHFQNSAQIAKALVNA |
| | | QVDFQAMWYTDQNHGIPGLSSKHLYTHMTHFLKQCFSLSE |
| 5 | >XP_040559378.1 | MCIVLLPSKVVTTVDGPRALTLDDYLNGNFQYKTYFPYWVSDSEYLHQNQ |
| | FAP | EDDIILFNVDMNYLTTIMTNSTMKQVNASNYVMSSDKYFIALESNYSKLW |
| | [organism = Gallus | RYSYTASYHIYDLIYGGFVTENQLPHKIQYISWSPVGHKLAYVYQNNIYLK |
| | gallus] | QSPREAPIKLTSDGKENEIFNGIPDWVYEEEMLATKYALWWSPSGKYLAY |
| | [GeneID = 424186] | VQFNDSDIPVIEYSYFGEDQYPRKIIIPYPKAGAKNPTVKVFIVDTTNIEAFG |
| | [isoform = X1] | PKEVPVPAVIASSDHYFTWLTWVTDSRVGVQWLKRIQNFSVLAICDFKENS |
| | | NTWDCPENQQHIEESQTGWAGGFFVSAPYFTSDGSSYYKIFSDKNGYKHIH |
| | | YINGSVENAIQITSGEWEAIYIFRVINDAIFYSSNEFEGYPGRRNIYKISIGSK |
| | | PIRKLCITCNLRKERCQYYTARFSERSKYYALICYGPGIPISTLFENESDREL |
| | | RILEDNQELQSALQEIILPKEEINKLEVDGITLWYKMLIPPQFDRSKKYPLLI |
| | | QVYGGPCSQNVKHTFSISWITYLASKEGIIVALVDGRGTAYQGDKILHAVY |
| | | RRLGVYEVEDQISAVKKFIEMGFIDEKRIAIWGWSYGGYVTSLALGSGSGV |
| | | FKCGIAVAPVSSWEYYASIYTERFMGLPVESDNLEHYKNSTVMARAKNFQ |
| | | NVEYLLIHGTADDNVHFQNSAQIAKALVNAQVDFQAMWYTDQNHGIPGL |
| | | SSKHLYTHMTHFLKQCFSLSE |
| 6 | >XP_046777356.1 | MCIVLLPSKVVTTVDGPRALTLDDYLNGNFQYKTYFPYWVSDSEYLHQNQ |
| | FAP | EDDIILFNVDMNYLTTIMTNSTMKQVNASNYVMSSDKYFIALESNYSKLW |
| | [organism = Gallus | RYSYTASYHIYDLIYGGFVTENQLPHKIQYISWSPVGHKLAYVYQNNIYLK |
| | gallus] | QSPREAPIKLTSDGKENEIFNGIPDWVYEEEMLATKYALWWSPSGKYLAY |
| | [GeneID = 424186] | VQFNDSDIPVIEYSYFGEDQYPRKIIIPYPKAGAKNPTVKVFIVDTTNIEAFG |
| | [isoform = X1] | PKEVPVPAVIASSDHYFTWLTWVTDSRVGVQWLKRIQNFSVLAICDFKENS |
| | | NTWDCPENQQHIEESQTGWAGGFFVSAPYFTSDGSSYYKIFSDKNGYKHIH |
| | | YINGSVENAIQITSGEWEAIYIFRVTNDAIFYSSNEFEGYPGRRNIYKISIGSK |
| | | PIRKLCITCNLRKERCQYYTARFSERSKYYALICYGPGIPISTLFETESDRELR |
| | | ILEDNQELQSALQEIILPKEEINKLEVDGITLWYKMLIPPQFDRSKKYPLLIQ |
| | | VYGGPCSQNVKHTFSISWITYLASKEGIIVALVDGRGTAYQGDKILHAVYR |
| | | RLGVYEVEDQISAVKKFIEMGFIDEKRIAIWGWSYGGYVTSLALGSGSGVF |
| | | KCGIAVAPVSSWEYYASIYTERFMGLPVESDNLEHYKNSTVMARAKNFQN |
| | | VEYLLIHGTADDNVHFQNSAQIAKALVNAQVDFQAMWYTDQNHGIPGLS |
| | | SKHLYTHMTHFLKQCFSLSE |
| 7 | >NP_001180568.2 | MKTRLKVLFGVVVCVLLSLLLMCIVLLPSKVVTTVDGPRALTLDDYLNGN |
| | FAP | FQYKTYFPYWVSDSEYLHQNQEDDIILFNVDMNYLTTIMTNSTMKQVNAS |
| | [organism = Gallus | NYVMSSDKYFIALESNYSKLWRYSYTASYHIYDLIYGGFVTENQLPHKIQY |
| | gallus] | ISWSPVGHKLAYVYQNNIYLKQSPREAPIKLTSDGKENEIFNGIPDWVYEEE |
| | [GeneID = 424186] | MLATKYALWWSPSGKYLAYVQFNDSDIPVIEYSYFGEDQYPRKIIIPYPKA |
| | | GAKNPTVKVFIVDTTNIEAFGPKEVPVPAVIASSDHYFTWLTWVTDSRVGV |
| | | QWLKRIQNFSVLAICDFKENSNTWDCPENQQHIEESQTGWAGGFFVSAPYF |
| | | TSDGSSYYKIFSDKNGYKHIHYINGSVENAIQITSGEWEAIYIFRVTNDAIFY |
| | | SSNEFEGYPGRRNIYKISIGSKPIRKLCITCNLRKERCQYYTARFSERSKYYA |
| | | LICYGPGIPISTLFENESDRELRILEDNQELQSALQEIILPKEEINKLEVDGITL |
| | | WYKMLIPPQFDRSKKYPLLIQVYGGPCSQNVKHTFSISWITYLASKEGIIVA |
| | | LVDGRGTAYQGDKILHAVYRRLGVYEVEDQISAVKKFIEMGFIDEKRIAIW |
| | | GWSYGGYVTSLALGSGSGVFKCGIAVAPVSSWEYYASIYTERFMGLPVES |
| | | DNLEHYKNSTVMARAKNFQNVEYLLIHGTADDNVHFQNSAQIAKALVNA |
| | | QVDFQAMWYTDQNHGIPGLSSKHLYTHMTHFLKQCFSLSE |
| 8 | >XP_040559378.1 | MCIVLLPSKVVTTVDGPRALTLDDYLNGNFQYKTYFPYWVSDSEYLHQNQ |
| | FAP | EDDIILFNVDMNYLTTIMTNSTMKQVNASNYVMSSDKYFIALESNYSKLW |
| | [organism = Gallus | RYSYTASYHIYDLIYGGFVTENQLPHKIQYISWSPVGHKLAYVYQNNIYLK |
| | gallus] | QSPREAPIKLTSDGKENEIFNGIPDWVYEEEMLATKYALWWSPSGKYLAY |
| | [GeneID = 424186] | VQFNDSDIPVIEYSYFGEDQYPRKIIIPYPKAGAKNPTVKVFIVDTTNIEAFG |
| | [isoform = X1] | PKEVPVPAVIASSDHYFTWLTWVTDSRVGVQWLKRIQNFSVLAICDFKENS |
| | | NTWDCPENQQHIEESQTGWAGGFFVSAPYFTSDGSSYYKIFSDKNGYKHIH |
| | | YINGSVENAIQITSGEWEAIYIFRVTNDAIFYSSNEFEGYPGRRNIYKISIGSK |
| | | PIRKLCITCNLRKERCQYYTARFSERSKYYALICYGPGIPISTLFENESDREL |
| | | RILEDNQELQSALQEIILPKEEINKLEVDGITLWYKMLIPPQFDRSKKYPLLI |
| | | QVYGGPCSQNVKHTFSISWITYLASKEGIIVALVDGRGTAYQGDKILHAVY |
| | | RRLGVYEVEDQISAVKKFIEMGFIDEKRIAIWGWSYGGYVTSLALGSGSGV |
| | | FKCGIAVAPVSSWEYYASIYTERFMGLPVESDNLEHYKNSTVMARAKNFQ |
| | | NVEYLLIHGTADDNVHFQNSAQIAKALVNAQVDFQAMWYTDQNHGIPGL |
| | | SSKHLYTHMTHFLKQCFSLSE |
| 9 | >XP_046777356.1 | MCIVLLPSKVVTTVDGPRALTLDDYLNGNFQYKTYFPYWVSDSEYLHQNQ |
| | FAP | EDDIILFNVDMNYLTTIMTNSTMKQVNASNYVMSSDKYFIALESNYSKLW |
| | [organism = Gallus | RYSYTASYHIYDLIYGGFVTENQLPHKIQYISWSPVGHKLAYVYQNNIYLK |
| | gallus] | QSPREAPIKLTSDGKENEIFNGIPDWVYEEEMLATKYALWWSPSGKYLAY |
| | [GeneID = 424186] | VQFNDSDIPVIEYSYFGEDQYPRKIIIPYPKAGAKNPTVKVFIVDTTNIEAFG |
| | [isoform = X1] | PKEVPVPAVIASSDHYFTWLTWVTDSRVGVQWLKRIQNFSVLAICDFKENS |
| | | NTWDCPENQQHIEESQTGWAGGFFVSAPYFTSDGSSYYKIFSDKNGYKHIH |
| | | YINGSVENAIQITSGEWEAIYIFRVINDAIFYSSNEFEGYPGRRNIYKISIGSK |
| | | PIRKLCITCNLRKERCQYYTARFSERSKYYALICYGPGIPISTLFETESDRELR |
| | | ILEDNQELQSALQEIILPKEEINKLEVDGITLWYKMLIPPQFDRSKKYPLLIQ |
| | | VYGGPCSQNVKHTFSISWITYLASKEGIIVALVDGRGTAYQGDKILHAVYR |
| | | RLGVYEVEDQISAVKKFIEMGFIDEKRIAIWGWSYGGYVTSLALGSGSGVF |
| | | KCGIAVAPVSSWEYYASIYTERFMGLPVESDNLEHYKNSTVMARAKNFQN |
| | | VEYLLIHGTADDNVHFQNSAQIAKALVNAQVDFQAMWYTDQNHGIPGLS |
| | | SKHLYTHMTHFLKQCFSLSE |
| 10 | >XP_005671941.2 | MKTWLKIVFGVATSAVLALLVMCIVLRPSRVPNSEGSKTRALTLKDILNGT |
| | FAP | FSYKTFFPNWISGQEYLHQSTDDNVIFYNIETGESYTILSNTTMKSVNASSY |
| | [organism = Sus | GLSPDRQFAYLESDYSKLWRYSYTATYHIYDLRNGEFITRNELPRPIQYLC |
| | scrofa] | WSPVGSKLAYVYQNNIYLKQRPEDPPFQITYNGKENKIFNGIPDWVYEEEM |
| | [GeneID = 100736 | LATKYALWWSPNGRFLAYAEFNDTEIPVIAYSYYGDEQYPRTINIPYPKAG |
| | 572] | AKNPFVRIFIIDTSYPGHVGPREVPVPAMIASSDYYFSWFTWVTDDRICLQ |
| | [isoform = X1] | WLKRIQNVSVLSICDFREDWQTWNCPKTQEHIEESRTGWAGGFFVSTPVFS |
| | | YDAISYYKIFSDKDGYKHIHYIKDSVENAIQITSGKWEAINIFRVTQDSLFYS |
| | | SNEFEGYPGRRNIYRISIGSHPPSKKCVTCHLREKRCQYYTASFSDYAKYYA |
| | | LVCYGPGLPISTLHDGRTDQEIKILEENKDLEYALKNIRLPKEEIKKLDVDDI |
| | | TLWYKMILPPQFDRSKKYPLLIQVYGGPCSQSVRSVFSIRWISYLASKEGIVI |
| | | ALVDGRGTAFQGDKLLYAVYRKLGVYEVEDQITAVRKFIEMGFIDEKRIAI |
| | | WGWSYGGYVSSLALASGTGLFKCGIAVAPVSSWEYYASIYTERFMGLPTK |
| | | DDNLEHYKNSTVMARAEYFRNVDYLLIHGTADDNVHFQNSAQIAKALVN |
| | | AQVDFQAMWYSDQNHGISGLSTKHLYTHMTHFLKQCFSLPD |
| 11 | >XP_020930559.1 | MCIVLRPSRVPNSEGSKTRALTLKDILNGTFSYKTFFPNWISGQEYLHQSTD |
| | FAP | DNVIFYNIETGESYTILSNTTMKSVNASSYGLSPDRQFAYLESDYSKLWRYS |
| | [organism = Sus | YTATYHIYDLRNGEFITRNELPRPIQYLCWSPVGSKLAYVYQNNIYLKQRP |
| | scrofa] | EDPPFQITYNGKENKIFNGIPDWVYEEEMLATKYALWWSPNGRFLAYAEF |
| | [GeneID = 100736 | NDTEIPVIAYSYYGDEQYPRTINIPYPKAGAKNPFVRIFIIDTSYPGHVGPRE |
| | 572] | VPVPAMIASSDYYFSWFTWVTDDRICLQWLKRIQNVSVLSICDFREDWQT |
| | [isoform = X2] | WNCPKTQEHIEESRTGWAGGFFVSTPVFSYDAISYYKIFSDKDGYKHIHYIK |
| | | DSVENAIQITSGKWEAINIFRVTQDSLFYSSNEFEGYPGRRNIYRISIGSHPPS |
| | | KKCVTCHLREKRCQYYTASFSDYAKYYALVCYGPGLPISTLHDGRTDQEI |
| | | KILEENKDLEYALKNIRLPKEEIKKLDVDDITLWYKMILPPQFDRSKKYPLL |
| | | IQVYGGPCSQSVRSVFSIRWISYLASKEGIVIALVDGRGTAFQGDKLLYAVY |
| | | RKLGVYEVEDQITAVRKFIEMGFIDEKRIAIWGWSYGGYVSSLALASGTGL |
| | | FKCGIAVAPVSSWEYYASIYTERFMGLPTKDDNLEHYKNSTVMARAEYFR |
| | | NVDYLLIHGTADDNVHFQNSAQIAKALVNAQVDFQAMWYSDQNHGISGL |
| | | STKHLYTHMTHFLKQCFSLPD |
| 12 | >NP_001354556.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 13 | >NP_001354557.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 14 | >NP_001354558.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 15 | >NP_001354559.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 16 | >NP_001354560.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 17 | >NP_001354561.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 18 | >NP_776512.2 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| 19 | >XP_005227327.1 | MVSPDPQMTVVAPGAEPESTQVQRIEDGGTIIRIFWVGPKGELLRRTPVSSV |
| | IGF2 | LQTSMGITAGKSVLVLLAFLAFASCCYAAYRPSETLCGGELVDTLQFVCGD |
| | [organism = Bos | RGFYFSRPSSRINRRSRGIVEECCFRSCDLALLETYCATPAKSERDVSASTTV |
| | taurus] | LPDDVTAYPVGKFFQYDIWKQSTQRLRRGLPAFLRARRGRTLAKELEALR |
| | [GeneID = 281240] | EAKSHRPLIALPTQDPATHGGASSKASSD |
| | [isoform = X1] | |
| 20 | >XP_005227329.1 | MVSPDPQMTVVAPGAEPESTQVQRIEDGGTIIRIFWVGPKGELLRRTPVSSV |
| | IGF2 | LQTSMGITAGKSVLVLLAFLAFASCCYAAYRPSETLCGGELVDTLQFVCGD |
| | [organism = Bos | RGFYFSRPSSRINRRSRGIVEECCFRSCDLALLETYCATPAKSERDVSASTTV |
| | taurus] | LPDDVTAYPVGKFFQYDIWKQSTQRLRRGLPAFLRARRGRTLAKELEALR |
| | [GeneID = 281240] | EAKSHRPLIALPTQDPATHGGASSKASSD |
| | [isoform = X1] | |
| 21 | >XP_005227330.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 22 | >XP_005227331.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 23 | >XP_005227332.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 24 | >XP_005227333.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 25 | >XP_015316818.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 26 | >XP_024842737.1 | MGITAGKSVLVLLAFLAFASCCYAAYRPSETLCGGELVDTLQFVCGDRGF |
| | IGF2 | YFSRPSSRINRRSRGIVEECCFRSCDLALLETYCATPAKSERDVSASTTVLPD |
| | [organism = Bos | DVTAYPVGKFFQYDIWKQSTQRLRRGLPAFLRARRGRTLAKELEALREAK |
| | taurus] | SHRPLIALPTQDPATHGGASSKASSD |
| | [GeneID = 281240] | |
| | [isoform = X2] | |
| 27 | >NP_001025513.1 | MCAARQILLLLLAFLAYALDSAAAYGTAETLCGGELVDTLQFVCGDRGFY |
| | IGF2 | FSRPVGRNNRRINRGIVEECCFRSCDLALLETYCAKSVKSERDLSATSLAGL |
| | [organism = Gallus | PALNKESFQKPSHAKYSKYNVWQKKSSQRLQREVPGILRARRYRWQAEG |
| | gallus] | LQAAEEARAMHRPLISLPSQRPPAPRASPEATGPQE |
| | [GeneID = 395097] | |
| 28 | >NP_999048.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| 29 | >XP_020936234.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 30 | >XP_020936235.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2] | |
| 31 | >XP_020936241.1 | MVSPDPQIIVVAPGAEPASTQGQRAEDGGTIIRIFWVGPQSELLRRAPVSSV |
| | IGF2 | LQIPMGIPMRKPLLVLLVFLALASCCYAAYRPSETLCGGELVDTLQFVCGD |
| | [organism = Sus | RGFYFRLPGRPASRVNRRSRGIVEECCFRSCDLALLETYCATPAKSERDVST |
| | scrofa] | PPTVLPDNFPRYPVGKFFRYDTWKQSAQRLRRGLPALLRARRGRTLAKEL |
| | [GeneID = 396916] | EAVREAKRHRPLTARPTRDPAAHGGASPEASGHRK |
| | [isoform = X3] | |
| 32 | >XP_020936244.1 | MVSPDPQIIVVAPGAEPASTQGQRAEDGGTIIRIFWVGPQSELLRRAPVSSV |
| | IGF2 | LQIPMGIPMRKPLLVLLVFLALASCCYAAYRPSETLCGGELVDTLQFVCGD |
| | [organism = Sus | RGFYFSRPASRVNRRSRGIVEECCFRSCDLALLETYCATPAKSERDVSTPPT |
| | scrofa] | VLPDNFPRYPVGKFFRYDTWKQSAQRLRRGLPALLRARRGRTLAKELEAV |
| | [GeneID = 396916] | REAKRHRPLTARPTRDPAAHGGASPEASGHRK |
| | [isoform = X4] | |
| 33 | >XP_020936252.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 34 | >XP_020936255.1 | MIDEGRSRARKPQLDFRGGAGGASCLRLEQIPMGIPMRKPLLVLLVFLALA |
| | IGF2 | SCCYAAYRPSETLCGGELVDTLQFVCGDRGFYFSRPASRVNRRSRGIVEEC |
| | [organism = Sus | CFRSCDLALLETYCATPAKSERDVSTPPTVLPDNFPRYPVGKFFRYDTWKQ |
| | scrofa] | SAQRLRRGLPALLRARRGRTLAKELEAVREAKRHRPLTARPTRDPAAHGG |
| | [GeneID = 396916] | ASPEASGHRK |
| | [isoform = X5] | |
| 35 | >XP_020936262.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 36 | >XP_020936266.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 37 | >XP_020936271.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 38 | >XP_020936275.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 39 | >XP_020936280.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2] | |
| 40 | >XP_020936291.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2 | |
| 41 | >XP_020936296.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2] | |
| 42 | >XP_020936300.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2] | |
| 43 | >XP_020936302.1 | MGIPMRKPLLVLLVFLALASCCYAAYRPSETLCGGELVDTLQFVCGDRGF |
| | IGF2 | YFRLPGRPASRVNRRSRGIVEECCFRSCDLALLETYCATPAKSERDVSTPPT |
| | [organism = Sus | VLPDNFPRYPVGKFFRYDTWKQSAQRLRRGLPALLRARRGRTLAKELEAV |
| | scrofa] | REAKRHRPLTARPTRDPAAHGGASPEASGHRK |
| | [GeneID = 396916] | |
| | [isoform = X1] | |
| 44 | >XP_020936307.1 | MGIPMRKPLLVLLVFLALASCCYAAYRPSETLCGGELVDTLQFVCGDRGF |
| | IGF2 | YFSRPASRVNRRSRGIVEECCFRSCDLALLETYCATPAKSERDVSTPPTVLP |
| | [organism = Sus | DNFPRYPVGKFFRYDTWKQSAQRLRRGLPALLRARRGRTLAKELEAVREA |
| | scrofa] | KRHRPLTARPTRDPAAHGGASPEASGHRK |
| | [GeneID = 396916] | |
| | [isoform = X2] | |
| 45 | >XP_002692376.11 | MAPARLLALLLLLVGVPAAAAESIRETEVIDPQDLLESQYFSGDLPDDEDV |
| | SDC4 | GGPGQESDDFELSGSGDLDDSEDIRIFPEVMHPLVPIDNHIPEKTGSGVPTEP |
| | [organism = Bos | KELEENEVIPKRSSPFDGDEDVSNKVSMSSTAQGSNIFERTEVLAALVVGG |
| | taurus] | VVGILFAVFLVLLLVYRMKKKDEGSYDLGKKPIYKKAPTNEFYA |
| | [GeneID = 508133] | |
| | [isoform = X2] | |
| 46 | >XP_024857036. | MAPARLLALLLLLVGVPAAAAESVPVIVSKALGPPEAPFIPTIRETEVIDPQD |
| | SDC4 | LLESQYFSGDLPDDEDVGGPGQESDDFELSGSGDLDDSEDIRIFPEVMHPLV |
| | [organism = Bos | PIDNHIPEKTGSGVPTEPKELEENEVIPKRSSPFDGDEDVSNKVSMSSTAQG |
| | taurus] | SNIFERTEVLAALVVGGVVGILFAVFLVLLLVYRMKKKDEGSYDLGKKPIY |
| | [GeneID = 508133] | KKAPTNEFYA |
| | [isoform = X1] | |
| 47 | >NP_001007870.1 | MPLPRAAFLLGLLLAAAAAESVRETETMDARWLDNVGSGDLPDDEDIGEF |
| | SDC4 | TPHLTSDEFDIDDTSGSGDYSDYDDAIYLTTVDTPAISDNYIPGDTERKMEG |
| | [organism = Gallus | EKKNTMLDNEIIPDKASPVEANLSNKISMASTANSSIFERTEVLTALIAGGA |
| | gallus] | VGLLFAVFLILLLVYRMKKKDEGSYDLGKKPIYKKAPTNEFYA |
| | [GeneID = 419184] | |
| 48 | >XP_040506606.1 | MEALCSAQVRETETMDARWLDNVGSGDLPDDEDIGEFTPHLTSDEFDIDD |
| | SDC4 | TSGSGDYSDYDDAIYLTTVDTPAISDNYIPGDTERKMEGEKKNTMLDNEIIP |
| | [organism = Gallus | DKASPVEANLSNKISMASTANSSIFERTEVLTALIAGGAVGLLFAVFLILLL |
| | gallus] | VYRMKKKDEGSYDLGKKPIYKKAPTNEFYA |
| | [GeneID = 419184] | |
| | [isoform = X2] | |
| 49 | >XP_046758781.1 | MCLGSGFALESRGEVRETETMDARWLDNVGSGDLPDDEDIGEFTPHLTSD |
| | SDC4 | EFDIDDTSGSGDYSDYDDAIYLTTVDTPAISDNYIPGDTERKMEGEKKNTM |
| | [organism = Gallus | LDNEIIPDKASPVEANLSNKISMASTANSSIFERTEVLTALIAGGAVGLLFAV |
| | gallus] | FLILLLVYRMKKKDEGSYDLGKKPIYKKAPTNEFYA |
| | [GeneID = 419184] | |
| | [isoform = X1] | |
| 50 | >XP_046758782.1 | MDARWLDNVGSGDLPDDEDIGEFTPHLTSDEFDIDDTSGSGDYSDYDDAI |
| | SDC4 | YLTTVDTPAISDNYIPGDTERKMEGEKKNTMLDNEIIPDKASPVEANLSNKI |
| | [organism = Gallus | SMASTANSSIFERTEVLTALIAGGAVGLLFAVFLILLLVYRMKKKDEGSYD |
| | gallus] | LGKKPIYKKAPTNEFYA |
| | [GeneID = 419184] | |
| | [isoform = X3] | |
| 51 | >XP_046758783.1 | MDARWLDNVGSGDLPDDEDIGEFTPHLTSDEFDIDDTSGSGDYSDYDDAI |
| | SDC4 | YLTTVDTPAISDNYIPGDTERKMEGEKKNTMLDNEIIPDKASPVEANLSNKI |
| | [organism = Gallus | SMASTANSSIFERTEVLTALIAGGAVGLLFAVFLILLLVYRMKKKDEGSYD |
| | gallus] | LGKKPIYKKAPTNEFYA |
| | [GeneID = 419184] | |
| | [isoform = X3] | |
| 52 | >XP_046786612.1 | MCLGSGFALESSGEVRETETMDARWLDNVGSGDLPDDEDIGEFTPHLTSD |
| | SDC4 | EFDIDDTSGSGDYSDYDDAIYLTTVDTPAISDNYIPGDTERKMEGEKKNTM |
| | [organism = Gallus | LDNEIIPDKASPVEANLSNKISMASTANSSIFERTEVLTALIAGGAVGLLFAV |
| | gallus] | FLILLLVYRMKKKDEGSYDLGKKPIYKKAPTNEFYA |
| | [GeneID = 419184] | |
| | [isoform = X1] | |
| 53 | >XP_046786613.1 | MEALCSAQVRETETMDARWLDNVGSGDLPDDEDIGEFTPHLTSDEFDIDD |
| | SDC4 | TSGSGDYSDYDDAIYLTTVDTPAISDNYIPGDTERKMEGEKKNTMLDNEIIP |
| | [organism = Gallus | DKASPVEANLSNKISMASTANSSIFERTEVLTALIAGGAVGLLFAVFLILLL |
| | gallus] | VYRMKKKDEGSYDLGKKPIYKKAPTNEFYA |
| | [GeneID = 419184] | |
| | [isoform = X2] | |
| 54 | >XP_046786615.1 | MDARWLDNVGSGDLPDDEDIGEFTPHLTSDEFDIDDTSGSGDYSDYDDAI |
| | SDC4 | YLTTVDTPAISDNYIPGDTERKMEGEKKNTMLDNEIIPDKASPVEANLSNKI |
| | [organism-Gallus | SMASTANSSIFERTEVLTALIAGGAVGLLFAVFLILLLVYRMKKKDEGSYD |
| | gallus] | LGKKPIYKKAPTNEFYA |
| | [GeneID = 419184] | |
| | [isoform = X3] | |
| 55 | >XP_046786616.1 | MDARWLDNVGSGDLPDDEDIGEFTPHLTSDEFDIDDTSGSGDYSDYDDAI |
| | SDC4 | YLTTVDTPAISDNYIPGDTERKMEGEKKNTMLDNEIIPDKASPVEANLSNKI |
| | [organism = Gallus | SMASTANSSIFERTEVLTALIAGGAVGLLFAVFLILLLVYRMKKKDEGSYD |
| | gallus] | LGKKPIYKKAPTNEFYA |
| | [GeneID = 419184] | |
| | [isoform = X3] | |
| 56 | >NP_999449.1 | MASPRLLALLLLLVGAFNAAAAESIRETEVINPQDLLEGRYFSGDLPDDED |
| | SDC4 | VGGPGQEPDDFEWSGSGDLEGPEDKHMLPEVTHPLVPLDNHIPERTGPGG |
| | [organism = Sus | RVPTEPKELEENEVIPKRMSPFDGDEDVSNKVSMSSTAQGSNIFERTEVLSA |
| | scrofa] | LIVGGIVGILFAVFLVLLLVYRMKKKDEGSYDLGKKPIYKKAPTNEFYA |
| | [GeneID = 397528] | |
| 57 | >XP_020932950.1 | MASPRLLALLLLLVGAFNAAAAESIRETEVINPQDLLEGRYFSGDLPDDED |
| | SDC4 | VGGPGQEPDDFEWSGSGDLEGPEDKHMLPEVTHPLVPLDNHIPERTGPGG |
| | [organism = Sus | RVPTEPKELEENEVIPKRMSPFDGDEDVSNKVSMSSTAQGSNIFERTEVLSG |
| | scrofa] | KVLSSPQDSSKQEGARRGIMGRLTLAHSAPWCPFLAAAGCPGALKADTFP |
| | [GeneID = 397528] | KT |
| | [isoform = X1] | |
| 58 | >XP_002696250.4 | MLGSPVRMGSPRPVYTAKLLSDPATAGNDAGAPTAPATGGEGEPHSRHRE |
| | SPHK1 | ARLGSTDKELKSGAAAADSVRTAPGTPRQRGEVMDAAGGSRNTLPRPCH |
| | [organism = Bos | VLVLLNPRGGKGKALQLFRSHVQPLLAQADVSFTLMLTERRNHARELVRA |
| | taurus] | EDLRRWDALVVMSGDGLIHEVVNGLMERPDWETAIQKPLCSLPAGSGNA |
| | [GeneID = 618605] | VAASLNYYAGYEQVTKEDLLTNCTQLLCRRLLAPMDLLSLQTACGQRLFS |
| | [isoform = X2] | VLSLAWGFIADVDLESEKFRRLGEMRFTLGACLRLVALRTYRGSLAYLPA |
| | | ETLASRRGPSPALAPQDPADTHVVPLEQPVPPHWTVVPEQDFVLVLALLHS |
| | | HLASDLFTAPMGRCSAGAMHLFYVRAGVSRAMLLRLFLAMEKGRHMEC |
| | | HCPHLVYVPVVAFRLEPKDGKGVFTVDGELLISEAVQGQVLPNYFWMVS |
| | | GYREPPPSLKPQPSPRRKSQQTLPPSERP |
| 59 | >XP_024836819.1 | MLGSPVRMGSPRPVYTAKLLSDPATAGNDAGAPTAPATGGEGEPHSRHRE |
| | SPHK1 | ARLGSTDKELKSGAAAADSVRTAPGTPRQRGEVMDAGAAFPPGCPSCDPQ |
| | [organism = Bos | PLVAVPVCVYSGRLPEHAPKALPRAGVVKSARGQGQSPAALQEPRTAAAG |
| | taurus] | PGRRLLHADAHCRALCTERRNHARELVRAEDLRRWDALVVMSGDGLIHE |
| | [GeneID-618605] | VVNGLMERPDWETAIQKPLCSLPAGSGNAVAASLNYYAGYEQVTKEDLL |
| | [isoform = X1] | TNCTQLLCRRLLAPMDLLSLQTACGQRLFSVLSLAWGFIADVDLESEKFRR |
| | | LGEMRFTLGACLRLVALRTYRGSLAYLPAETLASRRGPSPALAPQDPADTH |
| | | VVPLEQPVPPHWTVVPEQDFVLVLALLHSHLASDLFTAPMGRCSAGAMHL |
| | | FYVRAGVSRAMLLRLFLAMEKGRHMECHCPHLVYVPVVAFRLEPKDGKG |
| | | VFTVDGELLISEAVQGQVLPNYFWMVSGYREPPPSLKPQPSPRRKSQQTLP |
| | | PSERP |
| 60 | >XP_024836821.1 | MLGSPVRMGSPRPVYTAKLLSDPATAGNDAGAPTAPATGGEGEPHSRHRE |
| | SPHK1 | ARLGSTDKELKSGAAAADSVRTAPGTPRQRGEVMDAGAAFPPGCPSCDPQ |
| | [organism = Bos | PLVAVPVCVYSGRLPEHAPKALPRAGVVKSARGQGQSPAALQEPRTAAAG |
| | taurus] | PGRRLLHADAHCRALCTERRNHARELVRAEDLRRWDALVVMSGDGLIHE |
| | [GeneID = 618605] | VVNGLMERPDWETAIQKPLCSLPAGSGNAVAASLNYYAGYEQVTKEDLL |
| | [isoform = X1] | TNCTQLLCRRLLAPMDLLSLQTACGQRLFSVLSLAWGFIADVDLESEKFRR |
| | | LGEMRFTLGACLRLVALRTYRGSLAYLPAETLASRRGPSPALAPQDPADTH |
| | | VVPLEQPVPPHWTVVPEQDFVLVLALLHSHLASDLFTAPMGRCSAGAMHL |
| | | FYVRAGVSRAMLLRLFLAMEKGRHMECHCPHLVYVPVVAFRLEPKDGKG |
| | | VFTVDGELLISEAVQGQVLPNYFWMVSGYREPPPSLKPQPSPRRKSQQTLP |
| | | PSERP |
| 61 | >XP_024836822.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID-618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 62 | >XP_024836823.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 63 | >XP_024836824.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 64 | >XP_024836825.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 65 | >XP_024836826.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 66 | >XP_024836827.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 67 | >XP_024836828.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 68 | >XP_024836829.1 | MDAGAAFPPGCPSCDPQPLVAVPVCVYSGRLPEHAPKALPRAGVVKSARG |
| | SPHK1 | QGQSPAALQEPRTAAAGPGRRLLHADAHCRALCTERRNHARELVRAEDL |
| | [organism = Bos | RRWDALVVMSGDGLIHEVVNGLMERPDWETAIQKPLCSLPAGSGNAVAA |
| | taurus] | SLNYYAGYEQVTKEDLLTNCTQLLCRRLLAPMDLLSLQTACGQRLFSVLS |
| | [GeneID = 618605] | LAWGFIADVDLESEKFRRLGEMRFTLGACLRLVALRTYRGSLAYLPAETL |
| | [isoform = X3] | ASRRGPSPALAPQDPADTHVVPLEQPVPPHWTVVPEQDFVLVLALLHSHL |
| | | ASDLFTAPMGRCSAGAMHLFYVRAGVSRAMLLRLFLAMEKGRHMECHCP |
| | | HLVYVPVVAFRLEPKDGKGVFTVDGELLISEAVQGQVLPNYFWMVSGYR |
| | | EPPPSLKPQPSPRRKSQQTLPPSERP |
| 69 | >XP_024836831.1 | MDAAGGSRNTLPRPCHVLVLLNPRGGKGKALQLFRSHVQPLLAQADVSFT |
| | SPHK1 | LMLTERRNHARELVRAEDLRRWDALVVMSGDGLIHEVVNGLMERPDWE |
| | [organism = Bos | TAIQKPLCSLPAGSGNAVAASLNYYAGYEQVTKEDLLTNCTQLLCRRLLA |
| | taurus] | PMDLLSLQTACGQRLFSVLSLAWGFIADVDLESEKFRRLGEMRFTLGACLR |
| | [GeneID = 618605] | LVALRTYRGSLAYLPAETLASRRGPSPALAPQDPADTHVVPLEQPVPPHWT |
| | [isoform = X4] | VVPEQDFVLVLALLHSHLASDLFTAPMGRCSAGAMHLFYVRAGVSRAML |
| | | LRLFLAMEKGRHMECHCPHLVYVPVVAFRLEPKDGKGVFTVDGELLISEA |
| | | VQGQVLPNYFWMVSGYREPPPSLKPQPSPRRKSQQTLPPSERP |
| 70 | >XP_024836832.1 | MDAAGGSRNTLPRPCHVLVLLNPRGGKGKALQLFRSHVQPLLAQADVSFT |
| | SPHK1 | LMLTERRNHARELVRAEDLRRWDALVVMSGDGLIHEVVNGLMERPDWE |
| | [organism = Bos | TAIQKPLCSLPAGSGNAVAASLNYYAGYEQVTKEDLLTNCTQLLCRRLLA |
| | taurus] | PMDLLSLQTACGQRLFSVLSLAWGFIADVDLESEKFRRLGEMRFTLGACLR |
| | [GeneID = 618605] | LVALRTYRGSLAYLPAETLASRRGPSPALAPQDPADTHVVPLEQPVPPHWT |
| | [isoform = X4] | VVPEQDFVLVLALLHSHLASDLFTAPMGRCSAGAMHLFYVRAGVSRAML |
| | | LRLFLAMEKGRHMECHCPHLVYVPVVAFRLEPKDGKGVFTVDGELLISEA |
| | | VQGQVLPNYFWMVSGYREPPPSLKPQPSPRRKSQQTLPPSERP |
| 71 | >XP_015150786.1 | MAAGRRVPPHGDGGAVLLRGRFSPGPSPGPAAWSVALTERELQVLRPGGP |
| | SPHK1 | DAALRLADCVGSAAFPSGPAAACFSLVGYPFRKARWGSPARQRLQRTFRV |
| | [organism = Gallus | SQHPNAEENLRIAQAWSQRIRELSLPAAPTRDGVSYGLLPRPCRALVLLNP |
| | gallus] | QSGAGRALEDFQAVVQPMLADADIAATVFVTERPHHAQEKVRDEDLSQW |
| | [GeneID = 427803] | DVLVVMSGDGLLHEVLNGLMERPDWEEALQTPLCILPGGSGNALAASINY |
| | [isoform = X1] | YAGNDHVAKKKLLTNCTFILCKGLHAPMDLVSLSTASGKRFFSFLGFGWG |
| | | FIADVDIDSEKYRRLGNARFTLGTLQCLVRLRVYQGRLSYLPAATEPGTPR |
| | | DRKAPARRAAPTAGTEAPSGHGALPADTLLVPLGQPVPPHWVVVPEEEFV |
| | | CVYAIYQSHLGTNLLMAPAARLHDGCIHLFYVGAGISRVALLKIFLAMGR |
| | | GTHLDLNCPHLHCVPVRAFRLEPRGSAGIMTVDGEALACEPVQGQIHGHM |
| | | CRVVTAS |
| 72 | >XP_040542155.1 | MAAGRRVPPHGDGGAVLLRGRFSPGPSPGPAAWSVALTERELQVLRPGGP |
| | SPHK1 | DAALRLADCVGSAAFPSGPAAACFSLVGYPFRKARWGSPARQRLQRTFRV |
| | [organism = Gallus | SQHPNAEENLRIAQAWSQRIRELSLPAAPTRDGVSYGLLPRPCRALVLLNP |
| | gallus] | QSGAGRALEDFQAVVQPMLADADIAATVFVTERPHHAQEKVRDEDLSQW |
| | [GeneID = 427803] | DVLVVMSGDGLLHEVLNGLMERPDWEEALQTPLCILPGGSGNALAASINY |
| | [isoform = X1] | YAGNDHVAKKKLLTNCTFILCKGLHAPMDLVSLSTASGKRFFSFLGFGWG |
| | | FIADVDIDSEKYRRLGNARFTLGTLQCLVRLRVYQGRLSYLPAATEPGTPR |
| | | DRKAPARRAAPTAGTEAPSGHGALPADTLLVPLGQPVPPHWVVVPEEEFV |
| | | CVYAIYQSHLGTNLLMAPAARLHDGCIHLFYVGAGISRVALLKIFLAMGR |
| | | GTHLDLNCPHLHCVPVRAFRLEPRGSAGIMTVDGEALACEPVQGQIHGHM |
| | | CRVVTAS |
| 73 | >XP_046758083.1 | MLADADIAATVFVTERPHHAQEKVRDEDLSQWDVLVVMSGDGLLHEVLN |
| | SPHK1 | GLMERPDWEEALQTPLCILPGGSGNALAASINYYAGNDHVAKKKLLTNCT |
| | [organism = Gallus | FILCKGLHAPMDLVSLSTASGKRFFSFLGFGWGFIADVDIDSEKYRRLGNA |
| | gallus] | RFTLGTLQCLVRLRVYQGRLSYLPAATEPGTPRDRKAPARRAAPTAGTEAP |
| | [GeneID = 427803] | SGHGALPADTLLVPLGQPVPPHWVVVPEEEFVCVYAIYQSHLGTNLLMAP |
| | [isoform = X2] | AARLHDGCIHLFYVGAGISRVALLKIFLAMGRGTHLDLNCPHLHCVPVRAF |
| | | RLEPRGSAGIMTVDGEALACEPVQGQIHGHMCRVVTAS |
| 74 | >XP_046758084.1 | MLADADIAATVFVTERPHHAQEKVRDEDLSQWDVLVVMSGDGLLHEVLN |
| | SPHK1 | GLMERPDWEEALQTPLCILPGGSGNALAASINYYAGNDHVAKKKLLTNCT |
| | [organism = Gallus | FILCKGLHAPMDLVSLSTASGKRFFSFLGFGWGFIADVDIDSEKYRRLGNA |
| | gallus] | RFTLGTLQCLVRLRVYQGRLSYLPAATEPGTPRDRKAPARRAAPTAGTEAP |
| | [GeneID = 427803] | SGHGALPADTLLVPLGQPVPPHWVVVPEEEFVCVYAIYQSHLGTNLLMAP |
| | [isoform = X2] | AARLHDGCIHLFYVGAGISRVALLKIFLAMGRGTHLDLNCPHLHCVPVRAF |
| | | RLEPRGSAGIMTVDGEALACEPVQGQIHGHMCRVVTAS |
| 75 | >XP_046785717.1 | MLADADIAATVFVTERPHHAQEKVRDEDLSQWDVLVVMSGDGLLHEVLN |
| | SPHK1 | GLMERPDWEEALQTPLCILPGGSGNALAASINYYAGNDHVAKKKLLTNCT |
| | [organism = Gallus | FILCKGLHAPMDLVSLSTASGKRFFSFLGFGWGFIADVDIDSEKYRRLGNA |
| | gallus] | RFTLGTLQCLVRLRVYQGRLSYLPAATEPGTPRDRKAPARRAAPTAGTEAP |
| | [GeneID = 427803] | SGHGALPADTLLVPLGQPVPPHWVVVPEEEFVCVYAIYQSHLGTNLLMAP |
| | [isoform = X2] | AARLHDGCIHLFYVGAGISRVALLKIFLAMGRGTHLDLNCPHLHCVPVRAF |
| | | RLEPRGSAGIMTVDGEALACEPVQGQIHGHMCRVVTAS |
| 76 | >XP_005656972.1 | MPERAAPRARSKDLAGRPLPQGAAPDATKAVSSPSDPATAGNDAGASTAP |
| | SPHK1 | APGGEGEPHSRHRDARLGSTDKELKAGTEAADSARTAPGTPWQRGPRVK |
| | [organism = Sus | VMDAAGGPRILLPRPCRVLVLLNPRGGKGKGLQLFRSHVQPLLVQADISFT |
| | scrofa] | LMLTDRRNHARELVRAEDLRLWDALVIMSGDGLMHEVVNGLMERPDWE |
| | [GeneID = 100519 | TAMQKPLCSLPAGSGNALAASLNHYAGYEQVTNEDLLTNCTRLLCRRVLS |
| | 210] | PMDLLSLQTASGLRVFSVLSLTWGFIADVDLESEKFRGLGEMRFTLGTFLR |
| | [isoform = X1] | LVTLRTYRGRLAYLPVETVVSKMPPASAPAQWAQQGPVDTHLVPLEEPVP |
| | | SHWTVVPEQDFVLVLALLHSHLGSEMFAAPMGRCTAGAMHLFYVRAGVS |
| | | RAMLLRLFLAMEKGRHMECECPHLVYVPVVAFRLEPKDGKGVFAVDGE |
| | | MLVSEAVQGQVHPNYFWMVSGCSESLPSREPQPSLSQKPQQRPPREERS |
| 77 | >XP_005656973.1 | MHGSPVRMRNPGSAYTTKLLSDPATAGNDAGASTAPAPGGEGEPHSRHR |
| | SPHK1 | DARLGSTDKELKAGTEAADSARTAPGTPWQRGPRVKVMDAAGGPRILLP |
| | [organism = Sus | RPCRVLVLLNPRGGKGKGLQLFRSHVQPLLVQADISFTLMLTDRRNHARE |
| | scrofa] | LVRAEDLRLWDALVIMSGDGLMHEVVNGLMERPDWETAMQKPLCSLPA |
| | [GeneID = 100519 | GSGNALAASLNHYAGYEQVTNEDLLTNCTRLLCRRVLSPMDLLSLQTASG |
| | 210] | LRVFSVLSLTWGFIADVDLESEKFRGLGEMRFTLGTFLRLVTLRTYRGRLA |
| | [isoform = X2] | YLPVETVVSKMPPASAPAQWAQQGPVDTHLVPLEEPVPSHWTVVPEQDFV |
| | | LVLALLHSHLGSEMFAAPMGRCTAGAMHLFYVRAGVSRAMLLRLFLAME |
| | | KGRHMECECPHLVYVPVVAFRLEPKDGKGVFAVDGEMLVSEAVQGQVHP |
| | | NYFWMVSGCSESLPSREPQPSLSQKPQQRPPREERS |
| 78 | >XP_005656975.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPDWET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 79 | >XP_020922248.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPD WET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 80 | >XP_020922249.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPDWET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 81 | >XP_020922250.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPD WET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 82 | >XP_020922251.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPDWET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 83 | >XP_020922252.1 | MDAAGGPRILLPRPCRVLVLLNPRGGKGKGLQLFRSHVQPLLVQADISFTL |
| | SPHK1 | MLTDRRNHARELVRAEDLRLWDALVIMSGDGLMHEVVNGLMERPD WET |
| | [organism = Sus | AMQKPLCSLPAGSGNALAASLNHYAGYEQVTNEDLLTNCTRLLCRRVLSP |
| | scrofa] | MDLLSLQTASGLRVFSVLSLTWGFIADVDLESEKFRGLGEMRFTLGTFLRL |
| | [GeneID = 100519 | VTLRTYRGRLAYLPVETVVSKMPPASAPAQWAQQGPVDTHLVPLEEPVPS |
| | 210] | HWTVVPEQDFVLVLALLHSHLGSEMFAAPMGRCTAGAMHLFYVRAGVSR |
| | [isoform = X3] | AMLLRLFLAMEKGRHMECECPHLVYVPVVAFRLEPKDGKGVFAVDGEML |
| | | VSEAVQGQVHPNYFWMVSGCSESLPSREPQPSLSQKPQQRPPREERS |
| 84 | >NP_001068718.2 | MAAAYLDPNLNHTPSSSTKTHLGTGVERSPGAMERVLKVFHYFESNSEPT |
| | PTK2 | TWASIIRHGDATDVRGIIQKIVDSHKVKHVACYGFRLSHLRSEEVHWLHLD |
| | [organism = Bos | MGVSNVREKYELAHPPEEWKYELRIRYLPKGFLNQFTEDKPTLNFFYQQV |
| | taurus] | KSDYMLEIADQVDQDIALKLGCLEIRRSYWEMRGNALEKKSNYEVLEKDV |
| | [GeneID = 506270] | GLKRFFPRSLLDSVKAKTLRKLIQQTFRQFANLNREESILKFFEILSPVYRFD |
| | | KECFKCALGSSWIISVELAIGPEEGISYLTDKGCNPTHLADFNQVQTIQYSN |
| | | SEDKDRKGTLQLKIAGAPEPLTVTAPSLTIAENMADLIDGYCRLVHGATQS |
| | | FIIRPQKEGERALPSIPKLANSEKQGVRTHAVSVSETDDYAEIIDEEDTYTMP |
| | | STRDYEIQRERIELGRCIGEGQFGDVHQGTYTSPENPALAVAIKTCKNCTSD |
| | | SVREKFLQEALTMRQFDHPHIVKLIGVITENPVWIIMELCTLGELRSFLQVR |
| | | KYSLDLASLILYAYQLSTALAYLESKRFVHRDIAARNVLVSSNDCVKLGDF |
| | | GLSRYMEDSTYYKGFTFSNYSSSSKGKLLIKWMAPESINFRRFTSASDVWM |
| | | XGVCMWEILMHGVKPFQGVKNNDVIGRIENGERLPMPPNCPPTLYSLMTK |
| | | CWAYDPSRRPRFTELKAQLSTILEEEKVQQEERMRMESRRQVTASWDSGG |
| | | SDEAPPKPSRPGYPSPRSSEGFYPSPQHTVQTNHYQVSGYPGPHGVTAMAG |
| | | SIYPGQASLLDQTDSWNHRPQEISMWQPSVEDSAALDLRGLGQALPTHLM |
| | | EERLIRQQQEMEEDQRWLEKEERFLKPDVRLSRGSIDREDGGPQGLTGNQ |
| | | HIYQPVGKPDPAAPPKKPPRPGAPGHLGSLASLGSPGDSYNEGVKLQPQEIS |
| | | PPPTANLDRSNDRVYENVTGLVRAVIEMSSKIQPAPPEEYVPMVKEVGLAL |
| | | RTLLATVDETIPVLPASTHREIEMAQKLLNSDLGELINKMKLAQQYVMTSL |
| | | QQEYKKQMLTAAHALAVDAKNLLDVIDQARLKALGQPRPH |
| 85 | >NP_001383479.1 | MLLLPGGAGRQAPQPAGAVCMEKSGCSPFPMCWAKEYDRYLASSKTMA |
| | PTK2 | AAYLDPNLNHTPSSSAKTHLGTGMERSPGAMERVLKVFHYFENSSEPTTW |
| | [organism = Gallus | ASIIRHGDATDVRGIIQKIVDCHKVKNVACYGLRLSHLQSEEVHWLHLDM |
| | gallus] | GVSNVREKFELAHPPEEWKYELRIRYLPKGFLNQFTEDKPTLNFFYQQVKN |
| | [GeneID = 396416] | DYMLEIADQVDQEIALKLGCLEIRRSYGEMRGNALEKKSNYEVLEKDVGL |
| | [isoform = a] | RRFFPKSLLDSVKAKTLRKLIQQTFRQFANLNREESILKFFEILSPVYRFDKE |
| | | CFKCALGSSWIISVELAIGPEEGISYLTDKGANPTHLADFNQVQTIQYSNSE |
| | | DKDRKGMLQLKIAGAPEPLTVTAPSLTIAENMADLIDGYCRLVNGATQSFII |
| | | RPQKEGERALPSIPKLANNEKQGVRSHTVSVSETDDYAEIIDEEDTYTMPST |
| | | RDYEIQRERIELGRCIGEGQFGDVHQGIYMSPENPAMAVAIKTCKNCTSDS |
| | | VREKFLQEALTMRQFDHPHIVKLIGVITENPVWIIMELCTLGELRSFLQVRK |
| | | FSLDLASLILYAYQLSTALAYLESKRFVHRDIAARNVLVSATDCVKLGDFG |
| | | LSRYMEDSTYYKASKGKLPIKWMAPESINFRRFTSASDVWMFGVCMWEIL |
| | | MHGVKPFQGVKNNDVIGRIENGERLPMPPNCPPTLYSLMTKCWAYDPSRR |
| | | PRFTELKAQLSTILEEEKLQQEERMRMESRRQVTVSWDSGGSDEAPPKPSR |
| | | PGYPSPRSSEGFYPSPQHMVQPNHYQVSGYSGSHGIPAMAGSIYPGQASLL |
| | | DQTDSWNHRPQEVSAWQPNMEDSGTLDVRGMGQVLPTHLMEERLIRQQ |
| | | QEMEEDQRWLEKEERFLKPDVRLSRGSIEREDGGLQGPAGNQHIYQPVGK |
| | | PDHAAPPKKPPRPGAPHLGSLASLNSPVDSYNEGVKIKPQEISPPPTANLDR |
| | | SNDKVYENVTGLVKAVIEMSSKIQPAPPEEYVPMVKEVGLALRTLLATVD |
| | | ESLPVLPASTHREIEMAQKLLNSDLAELINKMKLAQQYVMTSLQQEYKKQ |
| | | MLTAAHALAVDAKNLLDVIDQARLKMISQSRPH |
| 86 | >NP_001383480.1 | MDTPCAVHQCNLREMKRIWPTLIRAVRYLQSKKEYDRYLASSKTMAAAY |
| | PTK2 | LDPNLNHTPSSSAKTHLGTGMERSPGAMERVLKVFHYFENSSEPTTWASII |
| | [organism = Gallus | RHGDATDVRGIIQKIVDCHKVKNVACYGLRLSHLQSEEVHWLHLDMGVS |
| | gallus] | NVREKFELAHPPEEWKYELRIRYLPKGFLNQFTEDKPTLNFFYQQVKNDY |
| | [GeneID = 396416] | MLEIADQVDQEIALKLGCLEIRRSYGEMRGNALEKKSNYEVLEKDVGLRR |
| | [isoform = b] | FFPKSLLDSVKAKTLRKLIQQTFRQFANLNREESILKFFEILSPVYRFDKECF |
| | | KCALGSSWIISVELAIGPEEGISYLTDKGANPTHLADFNQVQTIQYSNSEDK |
| | | DRKGMLQLKIAGAPEPLTVTAPSLTIAENMADLIDGYCRLVNGATQSFIIRP |
| | | QKEGERALPSIPKLANNEKQGVRSHTVSVSETDDYAEIIDEEDTYTMPSTR |
| | | DYEIQRERIELGRCIGEGQFGDVHQGIYMSPENPAMAVAIKTCKNCTSDSV |
| | | REKFLQEALTMRQFDHPHIVKLIGVITENPVWIIMELCTLGELRSFLQVRKF |
| | | SLDLASLILYAYQLSTALAYLESKRFVHRDIAARNVLVSATDCVKLGDFGL |
| | | SRYMEDSTYYKASKGKLPIKWMAPESINFRRFTSASDVWMFGVCMWEIL |
| | | MHGVKPFQGVKNNDVIGRIENGERLPMPPNCPPTLYSLMTKCWAYDPSRR |
| | | PRFTELKAQLSTILEEEKLQQEERMRMESRRQVTVSWDSGGSDEAPPKPSR |
| | | PGYPSPRSSEGFYPSPQHMVQPNHYQVSGYSGSHGIPAMAGSIYPGQASLL |
| | | DQTDSWNHRPQEVSAWQPNMEDSGTLDVRGMGQVLPTHLMEERLIRQQ |
| | | QEMEEDQRWLEKEERFLKPDVRLSRGSIEREDGGLQGPAGNQHIYQPVGK |
| | | PDHAAPPKKPPRPGAPHLGSLASLNSPVDSYNEGVKIKPQEISPPPTANLDR |
| | | SNDKVYENVTGLVKAVIEMSSKIQPAPPEEYVPMVKEVGLALRTLLATVD |
| | | ESLPVLPASTHREIEMAQKLLNSDLAELINKMKLAQQYVMTSLQQEYKKQ |
| | | MLTAAHALAVDAKNLLDVIDQARLKMISQSRPH |
| 87 | >NP_001383481.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = c] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSDEISGDETDDYAEIID |
| | | EEDTYTMPSKSYGIDEARDYEIQRERIELGRCIGEGQFGDVHQGIYMSPENP |
| | | AMAVAIKTCKNCTSDSVREKFLQEALTMRQFDHPHIVKLIGVITENPVWII |
| | | MELCTLGELRSFLQVRKFSLDLASLILYAYQLSTALAYLESKRFVHRDIAA |
| | | RNVLVSATDCVKLGDFGLSRYMEDSTYYKASKGKLPIKWMAPESINFRRF |
| | | TSASDVWMFGVCMWEILMHGVKPFQGVKNNDVIGRIENGERLPMPPNCP |
| | | PTLYSLMTKCWAYDPSRRPRFTELKAQLSTILEEEKLQQEERMRMESRRQ |
| | | VTVSWDSGGSDEAPPKPSRPGYPSPRSSEGFYPSPQHMVQPNHYQVSGYSG |
| | | SHGIPAMAGSIYPGQASLLDQTDSWNHRPQEVSAWQPNMEDSGTLDVRG |
| | | MGQVLPTHLMEERLIRQQQEMEEDQRWLEKEERFLKPDVRLSRGSIERED |
| | | GGLQGPAGNQHIYQPVGKPDHAAPPKKPPRPGAPHLGSLASLNSPVDSYN |
| | | EGVKIKPQEISPPPTANLDRSNDKVYENVTGLVKAVIEMSSKIQPAPPEEYV |
| | | PMVKEVGLALRTLLATVDESLPVLPASTHREIEMAQKLLNSDLAELINKMK |
| | | LAQQYVMTSLQQEYKKQMLTAAHALAVDAKNLLDVIDQARLKMISQSRP |
| | | H |
| 88 | >NP_001383482.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = d] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSETDDYAEIIDEEDTYT |
| | | MPSKSYGIDEARDYEIQRERIELGRCIGEGQFGDVHQGIYMSPENPAMAVA |
| | | IKTCKNCTSDSVREKFLQEALTMRQFDHPHIVKLIGVITENPVWIIMELCTL |
| | | GELRSFLQVRKFSLDLASLILYAYQLSTALAYLESKRFVHRDIAARNVLVS |
| | | ATDCVKLGDFGLSRYMEDSTYYKASKGKLPIKWMAPESINFRRFTSASDV |
| | | WMFGVCMWEILMHGVKPFQGVKNNDVIGRIENGERLPMPPNCPPTLYSL |
| | | MTKCWAYDPSRRPRFTELKAQLSTILEEEKLQQEERMRMESRRQVTVSWD |
| | | SGGSDEAPPKPSRPGYPSPRSSEGFYPSPQHMVQPNHYQVSGYSGSHGIPA |
| | | MAGSIYPGQASLLDQTDSWNHRPQEVSAWQPNMEDSGTLDVRGMGQVLP |
| | | THLMEERLIRQQQEMEEDQRWLEKEERFLKPDVRLSRGSIEREDGGLQGP |
| | | AGNQHIYQPVGKPDHAAPPKKPPRPGAPHLGSLASLNSPVDSYNEGVKIKP |
| | | QEISPPPTANLDRSNDKVYENVTGLVKAVIEMSSKIQPAPPEEYVPMVKEV |
| | | GLALRTLLATVDESLPVLPASTHREIEMAQKLLNSDLAELINKMKLAQQYV |
| | | MTSLQQEYKKQMLTAAHALAVDAKNLLDVIDQARLKMISQSRPH |
| 89 | >NP_001383483.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = e] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSETDDYAEIIDEEDTYT |
| | | MPSTRDYEIQRERIELGRCIGEGQFGDVHQGIYMSPENPAMAVAIKTCKNC |
| | | TSDSVREKFLQEALTMRQFDHPHIVKLIGVITENPVWIIMELCTLGELRSFL |
| | | QVRKFSLDLASLILYAYQLSTALAYLESKRFVHRDIAARNVLVSATDCVKL |
| | | GDFGLSRYMEDSTYYKASKGKLPIKWMAPESINFRRFTSASDVWMFGVC |
| | | MWEILMHGVKPFQGVKNNDVIGRIENGERLPMPPNCPPTLYSLMTKCWA |
| | | YDPSRRPRFTELKAQLSTILEEEKLQQEERMRMESRRQVTVSWDSGGSDEA |
| | | PPKPSRPGYPSPRSSEGFYPSPQHMVQPNHYQVSGYSGSHGIPAMAGSIYPG |
| | | QASLLDQTDSWNHRPQEVSAWQPNMEDSGTLDVRGMGQVLPTHLMEER |
| | | LIRQQQEMEEDQRWLEKEERFLKPDVRLSRGSIEREDGGLQGPAGNQHIYQ |
| | | PVGKPDHAAPPKKPPRPGAPHLGSLASLNSPVDSYNEGVKIKPQEISPPPTA |
| | | NLDRSNDKVYENVTGLVKAVIEMSSKIQPAPPEEYVPMVKEVGLALRTLL |
| | | ATVDESLPVLPASTHREIEMAQKLLNSDLAELINKMKLAQQYVMTSLQQE |
| | | YKKQMLTAAHALAVDAKNLLDVIDQARLKMISQSRPH |
| 90 | >NP_001383484.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = e] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSETDDYAEIIDEEDTYT |
| | | MPSTRDYEIQRERIELGRCIGEGQFGDVHQGIYMSPENPAMAVAIKTCKNC |
| | | TSDSVREKFLQEALTMRQFDHPHIVKLIGVITENPVWIIMELCTLGELRSFL |
| | | QVRKFSLDLASLILYAYQLSTALAYLESKRFVHRDIAARNVLVSATDCVKL |
| | | GDFGLSRYMEDSTYYKASKGKLPIKWMAPESINFRRFTSASDVWMFGVC |
| | | MWEILMHGVKPFQGVKNNDVIGRIENGERLPMPPNCPPTLYSLMTKCWA |
| | | YDPSRRPRFTELKAQLSTILEEEKLQQEERMRMESRRQVTVSWDSGGSDEA |
| | | PPKPSRPGYPSPRSSEGFYPSPQHMVQPNHYQVSGYSGSHGIPAMAGSIYPG |
| | | QASLLDQTDSWNHRPQEVSAWQPNMEDSGTLDVRGMGQVLPTHLMEER |
| | | LIRQQQEMEEDQRWLEKEERFLKPDVRLSRGSIEREDGGLQGPAGNQHIYQ |
| | | PVGKPDHAAPPKKPPRPGAPHLGSLASLNSPVDSYNEGVKIKPQEISPPPTA |
| | | NLDRSNDKVYENVTGLVKAVIEMSSKIQPAPPEEYVPMVKEVGLALRTLL |
| | | ATVDESLPVLPASTHREIEMAQKLLNSDLAELINKMKLAQQYVMTSLQQE |
| | | YKKQMLTAAHALAVDAKNLLDVIDQARLKMISQSRPH |
| 91 | >NP_001383485.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = e] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSETDDYAEIIDEEDTYT |
| | | MPSTRDYEIQRERIELGRCIGEGQFGDVHQGIYMSPENPAMAVAIKTCKNC |
| | | TSDSVREKFLQEALTMRQFDHPHIVKLIGVITENPVWIIMELCTLGELRSFL |
| | | QVRKFSLDLASLILYAYQLSTALAYLESKRFVHRDIAARNVLVSATDCVKL |
| | | GDFGLSRYMEDSTYYKASKGKLPIKWMAPESINFRRFTSASDVWMFGVC |
| | | MWEILMHGVKPFQGVKNNDVIGRIENGERLPMPPNCPPTLYSLMTKCWA |
| | | YDPSRRPRFTELKAQLSTILEEEKLQQEERMRMESRRQVTVSWDSGGSDEA |
| | | PPKPSRPGYPSPRSSEGFYPSPQHMVQPNHYQVSGYSGSHGIPAMAGSIYPG |
| | | QASLLDQTDSWNHRPQEVSAWQPNMEDSGTLDVRGMGQVLPTHLMEER |
| | | LIRQQQEMEEDQRWLEKEERFLKPDVRLSRGSIEREDGGLQGPAGNQHIYQ |
| | | PVGKPDHAAPPKKPPRPGAPHLGSLASLNSPVDSYNEGVKIKPQEISPPPTA |
| | | NLDRSNDKVYENVTGLVKAVIEMSSKIQPAPPEEYVPMVKEVGLALRTLL |
| | | ATVDESLPVLPASTHREIEMAQKLLNSDLAELINKMKLAQQYVMTSLQQE |
| | | YKKQMLTAAHALAVDAKNLLDVIDQARLKMISQSRPH |
| 92 | >NP_001383486.1 | MAAAYLDPNLNHTPSSSAKTHLGTGMERSPGAMERVLKVFHYFENSSEPT |
| | PTK2 | TWASIIRHGDATDVRGIIQKIVDCHKVKNVACYGLRLSHLQSEEVHWLHL |
| | [organism = Gallus | DMGVSNVREKFELAHPPEEWKYELRIRYLPKGFLNQFTEDKPTLNFFYQQ |
| | gallus] | VKNDYMLEIADQVDQEIALKLGCLEIRRSYGEMRGNALEKKSNYEVLEKD |
| | [GeneID = 396416] | VGLRRFFPKSLLDSVKAKTLRKLIQQTFRQFANLNREESILKFFEILSPVYRF |
| | [isoform = e] | DKECFKCALGSSWIISVELAIGPEEGISYLTDKGANPTHLADFNQVQTIQYS |
| | | NSEDKDRKGMLQLKIAGAPEPLTVTAPSLTIAENMADLIDGYCRLVNGAT |
| | | QSFIIRPQKEGERALPSIPKLANNEKQGVRSHTVSVSETDDYAEIIDEEDTYT |
| | | MPSTRDYEIQRERIELGRCIGEGQFGDVHQGIYMSPENPAMAVAIKTCKNC |
| | | TSDSVREKFLQEALTMRQFDHPHIVKLIGVITENPVWIIMELCTLGELRSFL |
| | | QVRKFSLDLASLILYAYQLSTALAYLESKRFVHRDIAARNVLVSATDCVKL |
| | | GDFGLSRYMEDSTYYKASKGKLPIKWMAPESINFRRFTSASDVWMFGVC |
| | | MWEILMHGVKPFQGVKNNDVIGRIENGERLPMPPNCPPTLYSLMTKCWA |
| | | YDPSRRPRFTELKAQLSTILEEEKLQQEERMRMESRRQVTVSWDSGGSDEA |
| | | PPKPSRPGYPSPRSSEGFYPSPQHMVQPNHYQVSGYSGSHGIPAMAGSIYPG |
| | | QASLLDQTDSWNHRPQEVSAWQPNMEDSGTLDVRGMGQVLPTHLMEER |
| | | LIRQQQEMEEDQRWLEKEERFLKPDVRLSRGSIEREDGGLQGPAGNQHIYQ |
| | | PVGKPDHAAPPKKPPRPGAPHLGSLASLNSPVDSYNEGVKIKPQEISPPPTA |
| | | NLDRSNDKVYENVTGLVKAVIEMSSKIQPAPPEEYVPMVKEVGLALRTLL |
| | | ATVDESLPVLPASTHREIEMAQKLLNSDLAELINKMKLAQQYVMTSLQQE |
| | | YKKQMLTAAHALAVDAKNLLDVIDQARLKMISQSRPH |
| 93 | >XP_020944479.1 | MEKSRCSPFPVCWAKEYDRYLASSKIMAAAYLDPNLNHTPNSSTKTHLGT |
| | PTK2 | GVERSPGAMDRVLKVFHYFESNSEPTTWASIIRHGDATDVRGIIQKIVDSH |
| | [organism = Sus | KVKHVACYGFRLSHLRSEEVHWLHLDMGVSSVREKYELAHPPEEWKYEL |
| | scrofa] | RIRYLPKGFLNQFTEDKPTLNFFYQQVKSDYMLEIADQVDQDIALKLGCLE |
| | [GeneID = 102167 | IRRSYWEMRGNALEKKSNYEVLEKDVGLKRFFPRSLLDSVKAKTLRKLIQ |
| | 153] | QTFRQFANLNREESILKFFEILSPVYRFDKECFKCALGSSWIISVELAIGPEEG |
| | [isoform = X1] | ISYLTDKGCNPTHLADFNQVQTIQYSNSEDKDRKGTLQLKIAGAPEPLTVT |
| | | APSLTIAENMADLIDGYCRLVNGATQSFIIRPQKEGERALPSIPKLANSEKQ |
| | | GVRTHAVSVSGVSHCHHKVKKARRFLPLVFCSHEPPSTDEISGDETDDYAE |
| | | IIDEEDTYTMPSKSYGIDEARDYEIQRERIELGRCIGEGQFGDVHQGAYTSP |
| | | ENPALAVAIKTCKNCTSDSVREKFLQEALTMRQFDHPHIVKLIGVITENPV |
| | | WIIMELCTLGELRSFLQVRKYSLDLASLILYAYQLSTALAYLESKRFVHRDI |
| | | AARNVLVSSNDCVKLGDFGLSRYMEDSTYYKASKGKLPIKWMAPESINFR |
| | | RFTSASDVWMFGVCMWEILMLGVKPFQGVKNNDVIGRIENGERLPMPPN |
| | | CPPTLYSLMTKCWAYDPSRRPRFTELKAQLSTILEEEKVQQEERMRMESRR |
| | | QVAVSWDSGGSDEAPPKPSRPGYPSPRSSEGFYPSPQHMVQTNHYQVSGY |
| | | PGPHGVTAMAGSIYPGQASLLDQTDSWSQRPQEMSVWQPSVEDSTALDLR |
| | | GLGQVLPAHLMEERLIRQQQEMEEDQRWLEKEERFLKPDVRLSRGSIDRE |
| | | DGGLQGPTGNQHIYQPVGKPDPAAPPKKPPRPGAPGHLASLGSLGSPGDSY |
| | | NEGVKPWRLQPQEISPPPTANLDRSNDRVYENVTGLVRAVIEMSSKIQPAP |
| | | PEEYVPMVKEVGLALRTLLATVDETIPVLPASTHREIEMAQKLLNSDLGELI |
| | | SKMKLAQQYVMTSLQQEYKKQMLTAAHALAVDAKNLLDVIDQARLKVL |
| | | GQARPH |
| 94 | >XP_020944480.1 | MEKSRCSPFPVCWAKEYDRYLASSKIMAAAYLDPNLNHTPNSSTKTHLGT |
| | PTK2 | GVERSPGAMDRVLKVFHYFESNSEPTTWASIIRHGDATDVRGIIQKIVDSH |
| | [organism = Sus | KVKHVACYGFRLSHLRSEEVHWLHLDMGVSSVREKYELAHPPEEWKYEL |
| | scrofa] | RIRYLPKGFLNQFTEDKPTLNFFYQQVKSDYMLEIADQVDQDIALKLGCLE |
| | [GeneID = 102167 | IRRSYWEMRGNALEKKSNYEVLEKDVGLKRFFPRSLLDSVKAKTLRKLIQ |
| | 153] | QTFRQFANLNREESILKFFEILSPVYRFDKECFKCALGSSWIISVELAIGPEEG |
| | [isoform = X2] | ISYLTDKGCNPTHLADFNQVQTIQYSNSEDKDRKGTLQLKIAGAPEPLTVT |
| | | APSLTIAENMADLIDGYCRLVNGATQSFIIRPQKEGERALPSIPKLANSEKQ |
| | | GVRTHAVSVSGVSHCHHKVKKARRFLPLVFCSHEPPSTDEISGDETDDYAE |
| | | IIDEEDTYTMPSKSYGIDEARDYEIQRERIELGRCIGEGQFGDVHQGAYTSP |
| | | ENPALAVAIKTCKNCTSDSVREKFLQEALTMRQFDHPHIVKLIGVITENPV |
| | | WIIMELCTLGELRSFLQVRKYSLDLASLILYAYQLSTALAYLESKRFVHRDI |
| | | AARNVLVSSNDCVKLGDFGLSRYMEDSTYYKASKGKLPIKWMAPESINFR |
| | | RFTSASDVWMFGVCMWEILMLGVKPFQGVKNNDVIGRIENGERLPMPPN |
| | | CPPTLYSLMTKCWAYDPSRRPRFTELKAQLSTILEEEKVQQEERMRMESRR |
| | | QVAVSWDSGGSDEAPPKPSRPGYPSPRSSEGFYPSPQHMVQTNHYQVSGY |
| | | PGPHGVTAMAGSIYPGQASLLDQTDSWSQRPQEMSVWQPSVEDSTALDLR |
| | | GLGQVLPAHLMEERLIRQQQEMEEDQRWLEKEERFLKPDVRLSRGSIDRE |
| | | DGGLQGPTGNQHIYQPVGKPDPAAPPKKPPRPGAPGHLASLGSLGSPGDSY |
| | | NEGVKLQPQEISPPPTANLDRSNDRVYENVTGLVRAVIEMSSKIQPAPPEEY |
| | | VPMVKEVGLALRTLLATVDETIPVLPASTHREIEMAQKLLNSDLGELISKM |
| | | KLAQQYVMTSLQQEYKKQMLTAAHALAVDAKNLLDVIDQARLKVLGQA |
| | | RPH |
| 95 | >XP_020944481.1 | MEKSRCSPFPVCWAKEYDRYLASSKIMAAAYLDPNLNHTPNSSTKTHLGT |
| | PTK2 | GVERSPGAMDRVLKVFHYFESNSEPTTWASIIRHGDATDVRGIIQKIVDSH |
| | [organism = Sus | KVKHVACYGFRLSHLRSEEVHWLHLDMGVSSVREKYELAHPPEEWKYEL |
| | scrofa] | RIRYLPKGFLNQFTEDKPTLNFFYQQVKSDYMLEIADQVDQDIALKLGCLE |
| | [GeneID = 102167 | IRRSYWEMRGNALEKKSNYEVLEKDVGLKRFFPRSLLDSVKAKTLRKLIQ |
| | 153] | QTFRQFANLNREESILKFFEILSPVYRFDKECFKCALGSSWIISVELAIGPEEG |
| | [isoform = X3] | ISYLTDKGCNPTHLADFNQVQTIQYSNSEDKDRKGTLQLKIAGAPEPLTVT |
| | | APSLTIAENMADLIDGYCRLVNGATQSFIIRPQKEGERALPSIPKLANSEKQ |
| | | GVRTHAVSVSGVSHCHHKVKKARRFLPLVFCSHEPPSTETDDYAEIIDEED |
| | | TYTMPSKSYGIDEARDYEIQRERIELGRCIGEGQFGDVHQGAYTSPENPAL |
| | | AVAIKTCKNCTSDSVREKFLQEALTMRQFDHPHIVKLIGVITENPVWIIMEL |
| | | CTLGELRSFLQVRKYSLDLASLILYAYQLSTALAYLESKRFVHRDIAARNV |
| | | LVSSNDCVKLGDFGLSRYMEDSTYYKASKGKLPIKWMAPESINFRRFTSAS |
| | | DVWMFGVCMWEILMLGVKPFQGVKNNDVIGRIENGERLPMPPNCPPTLYS |
| | | LMTKCWAYDPSRRPRFTELKAQLSTILEEEKVQQEERMRMESRRQVAVS |
| | | WDSGGSDEAPPKPSRPGYPSPRSSEGFYPSPQHMVQTNHYQVSGYPGPHG |
| | | VTAMAGSIYPGQASLLDQTDSWSQRPQEMSVWQPSVEDSTALDLRGLGQ |
| | | VLPAHLMEERLIRQQQEMEEDQRWLEKEERFLKPDVRLSRGSIDREDGGL |
| | | QGPTGNQHIYQPVGKPDPAAPPKKPPRPGAPGHLASLGSLGSPGDSYNEGV |
| | | KPWRLQPQEISPPPTANLDRSNDRVYENVTGLVRAVIEMSSKIQPAPPEEYV |
| | | PMVKEVGLALRTLLATVDETIPVLPASTHREIEMAQKLLNSDLGELISKMK |
| | | LAQQYVMTSLQQEYKKQMLTAAHALAVDAKNLLDVIDQARLKVLGQAR |
| | | PH |
| 96 | >XP_020944482.1 | MEKSRCSPFPVCWAKEYDRYLASSKIMAAAYLDPNLNHTPNSSTKTHLGT |
| | PTK2 | GVERSPGAMDRVLKVFHYFESNSEPTTWASIIRHGDATDVRGIIQKIVDSH |
| | [organism = Sus | KVKHVACYGFRLSHLRSEEVHWLHLDMGVSSVREKYELAHPPEEWKYEL |
| | scrofa] | RIRYLPKGFLNQFTEDKPTLNFFYQQVKSDYMLEIADQVDQDIALKLGCLE |
| | [GeneID = 102167 | IRRSYWEMRGNALEKKSNYEVLEKDVGLKRFFPRSLLDSVKAKTLRKLIQ |
| | 153] | QTFRQFANLNREESILKFFEILSPVYRFDKECFKCALGSSWIISVELAIGPEEG |
| | [isoform = X4] | ISYLTDKGCNPTHLADFNQVQTIQYSNSEDKDRKGTLQLKIAGAPEPLTVT |
| | | APSLTIAENMADLIDGYCRLVNGATQSFIIRPQKEGERALPSIPKLANSEKQ |
| | | GVRTHAVSVSGVSHCHHKVKKARRFLPLVFCSHEPPSTDEISGDETDDYAE |
| | | IIDEEDTYTMPSTRDYEIQRERIELGRCIGEGQFGDVHQGAYTSPENPALAV |
| | | AIKTCKNCTSDSVREKFLQEALTMRQFDHPHIVKLIGVITENPVWIIMELCT |
| | | LGELRSFLQVRKYSLDLASLILYAYQLSTALAYLESKRFVHRDIAARNVLV |
| | | SSNDCVKLGDFGLSRYMEDSTYYKASKGKLPIKWMAPESINFRRFTSASDV |
| | | WMFGVCMWEILMLGVKPFQGVKNNDVIGRIENGERLPMPPNCPPTLYSL |
| | | MTKCWAYDPSRRPRFTELKAQLSTILEEEKVQQEERMRMESRRQVAVSW |
| | | DSGGSDEAPPKPSRPGYPSPRSSEGFYPSPQHMVQTNHYQVSGYPGPHGVT |
| | | AMAGSIYPGQASLLDQTDSWSQRPQEMSVWQPSVEDSTALDLRGLGQVLP |
| | | AHLMEERLIRQQQEMEEDQRWLEKEERFLKPDVRLSRGSIDREDGGLQGP |
| | | TGNQHIYQPVGKPDPAAPPKKPPRPGAPGHLASLGSLGSPGDSYNEGVKP |
| | | WRLQPQEISPPPTANLDRSNDRVYENVTGLVRAVIEMSSKIQPAPPEEYVP |
| | | MVKEVGLALRTLLATVDETIPVLPASTHREIEMAQKLLNSDLGELISKMKL |
| | | AQQYVMTSLQQEYKKQMLTAAHALAVDAKNLLDVIDQARLKVLGQARP |
| | | H |
| 97 | pFS2-ggEF1- | TGGCACGACAGGTTTCCCGACTGGAAAGCGGGCAGTGAGCGCAACGCA |
| | ggIGF2- | ATTAATGTGAGTTAGCTCACTCATTAGGCACCCCAGGCTTTACACTTTA |
| | mPGK-Neo | TGCTTCCGGCTCGTATGTTGTGTGGAATTGTGAGCGGATAACAATTTCA |
| | (Promoters: | CACAGGAAACAGCTATGACCATGATTACGCCAAGCTTAAGCTTGCATG |
| | rodentia PGK | CCTGCAGGTCGACCTGCAGCGATCGAACTTGTTTATTGCAGCTTATAAT |
| | (mPGK) and | GGTTACAAATAAAGCAATAGCATCACAAATTTCACAAATAAAGCATTT |
| | gallus gallus | TTTTCACTGCATTCTAGTTGTGGTTTGTCCAAACTCATCAATGTATCTTA |
| | EF1 (ggEF1)) | TCATGTCTGGATCCCTTCTGAGGGCAGAGGAAGTCTTAATTCAGAAGAA |
| | | CTCGTCAAGAAGGCGATAGAAGGCGATGCGCTGCGAATCGGGAGCGGC |
| | | GATACCGTAAAGCACGAGGAAGCGGTCAGCCCATTCGCCGCCAAGCTC |
| | | TTCAGCAATATCACGGGTAGCCAACGCTATGTCCTGATAGCGGTCCGCC |
| | | ACACCCAGCCGGCCACAGTCGATGAATCCAGAAAAGCGGCCATTTTCC |
| | | ACCATGATATTCGGCAAGCAGGCATCGCCATGGGTCACGACGAGATCC |
| | | TCGCCGTCGGGCATGCTCGCCTTGAGCCTGGCGAACAGTTCGGCTGGCG |
| | | CGAGCCCCTGATGCTCTTCGTCCAGATCATCCTGATCGACAAGACCGGC |
| | | TTCCATCCGAGTACGTGCTCGCTCGATGCGATGTTTCGCTTGGTGGTCG |
| | | AATGGGCAGGTAGCCGGATCAAGCGTATGCAGCCGCCGCATTGCATCA |
| | | GCCATGATGGATACTTTCTCGGCAGGAGCAAGGTGAGATGACAGGAGA |
| | | TCCTGCCCCGGCACTTCGCCCAATAGCAGCCAGTCCCTTCCCGCTTCAG |
| | | TGACAACGTCGAGCACAGCTGCGCAAGGAACGCCCGTCGTGGCCAGCC |
| | | ACGATAGCCGCGCTGCCTCGTCTTGCAGTTCATTCAGGGCACCGGACAG |
| | | GTCGGTCTTGACAAAAAGAACCGGGCGCCCCTGCGCTGACAGCCGGAA |
| | | CACGGCGGCATCAGAGCAGCCGATTGTCTGTTGTGCCCAGTCATAGCCG |
| | | AATAGCCTCTCCACCCAAGCGGCCGGAGAACCTGCGTGCAATCCATCTT |
| | | GTTCAATCATGCCCATGGTGGCGGCACGTGAGGTAGGTCGAAAGGCCC |
| | | GGAGATGAGGAAGAGGAGAACAGCGCGGCAGACGTGCGCTTTTGAAG |
| | | CGTGCAGAATGCCGGGCCTCCGGAGGACCTTCGGGCGCCCGCCCCGCC |
| | | CCTGAGCCCGCCCCTGAGCCCGCCCCCGGACCCACCCTTCCCAGCTGCT |
| | | GAGCCCAGAAAGCGAAGGAGCAAAGCTGCTATTGGCCGCTGCCCCAAA |
| | | GGCCTACCCGCTTCCATTGCTCAGCGGTGCTGTCCATCTGCACGAGACT |
| | | AGTGAGACGTGCTACTTCCATTTGTCACGTCCTGCACGACGCGAGCTGC |
| | | GGGGCGGGGGGGAACTTCCTGACTAGGGGAGGAGTAGAAGGTGGCGC |
| | | GAAGGGGCCACCAAAGAACGGAGCCGGTTGGCGCTACCGGTGGATGTG |
| | | GAATGTGTGCGAGGCCAGAGGCCACTTGTGTAGCGCCAAGTGCCAGCG |
| | | GGGCTGCTAAAGCGCATGCTCCAGACTGCCTTGGGAAAAGCGCCTCCC |
| | | CTACCCGGTAGAATTGGTGGCGATGCCGAGCTCGGTACCAAGCTTAAG |
| | | TTTAAACGCTAGCAGTAGAATTCGGATCCTACCGTTCGTATAGCATACA |
| | | TTATACGAAGTTATGTCGACGATGTAGGTCACGGTCTCGAAGCCGCGGT |
| | | GCGGGTGCCAGGGCGTGCCCTTGGGCTCCCCGGGCGCGTACTCCACCTC |
| | | ACCCATCTGGTCCATCATGATGAACGGTCGAGGTGGCGGTAGTTGATCC |
| | | CGGCGAACGCGCGGCGCACCGGGAAGCCCTCGCCCTCGAAACCGCTGG |
| | | GCGCGGTGGTCACGGTGAGCACGGGACGTGCGACGGCGTCGGCGGGTG |
| | | CGGATACGCGGGGCAGCGTCAGCGGGTTCTCGACGGTCACGGCGGGCA |
| | | TGTCGACGGCGCGCCGATGCCATGAGTACACCCTGCTGGAATCACGAG |
| | | GGCTTTCACTGCTGCTGCCTCGTGGCTGCTCGCCGCCCCGGGTTCAGAA |
| | | GCGAAAGGCTGGGGCTGCCAGCACCTTCGGTGGCTTTCGTCAGCACCC |
| | | GTCAGCCGGGAACATGTGGCGTGGGGGCACTGCGCGCAGTTTGGAGCT |
| | | CTCAAGCAGAAGCCACGAATCCTTGCCGCTGAAGCGATTCTCTCTGGCC |
| | | GCATCTGAAACGCCAGGCCAGAGGAGGTCTGTGCAAGTAACTACTCCC |
| | | GGAGCTCGCTGACCGCCTCCCCTCGGGCCGCAGCCGCGGGTCAGAGGG |
| | | GCAGAGCTCGGGGACTCTACGGCTGCCGTGCTGCGCGGGAGGCGGCGG |
| | | AGCGGCAACGCCGTCCCGCCCTGCAGTGCTCCGGGCGCTGCCGTCCGG |
| | | CCTGGGGGCGGCGAGCCCCTCCCGGCAGTCGCTCGTGACTCCCGCCCG |
| | | ATCCGGCCGCCGCGGGGCTCTGCTCCTCAGCCCCGCCCCTCCCCGCCGC |
| | | TCCTCAGCGGAGCGCCGCGTGCCGCGCTGCCGGGCGGGGCGTTGCGCG |
| | | GGGGCCGCCGGGAGCGGTGACGCGTCGCCGAGGCCCCGCCCCGTCGCA |
| | | GAGTATATAAGGGGCGGCGCTCACGCCGTCGTTCTTTTCCGCTCCGGGT |
| | | TTGCCGACGCCGTGCGGGTGAGGCGCGTGCGGCGGGGCAGGCCGGGCC |
| | | CGGGGGTGGGGGGTGCGGGGTGCGGGGATGCTCGGGGGGAGCCTCGTT |
| | | GCACCGTGAGCGCGGGGCCGGGGCGCGGCCGCGGAGAGGCGCGTGGG |
| | | AACGGCGGAGTGGGGAGGGGGAAGCGAGATGAGGGACGGGTGGGAGA |
| | | GAGCGGGGCGAGCGCCGGGGTAAAGCCGACGTGTCCCGCCTGAGGACC |
| | | CACGCGGGGCAGCGCGGCGATGAGCACGCGGGGCCCCACCGCCGCCTT |
| | | GGCTTCGCGCGGAGCCGCCCGGCGCTGGGGCGCGGCGTACGGTGCTCG |
| | | GCCTCGGGCGGGTGGCGGCCGGGGACCCCGGGGCCGCGCCGTCGGTGC |
| | | CGCGGTGGGGGAGGGGAGGCGAGGCCGGAGCACGAGAAAAAAAATGG |
| | | CCGCCCGCTCTTCCTGCCGCCGCCAGCTCACAAAATGGAGGACGGCGG |
| | | CCCGGGCGGGGTGAGTCACACACAAAGGAGAGGGGCGGCCCCGCCCTC |
| | | CGCGCCGCCCGTGGCCGCGCCGCCGCCCCGCGCCCCCACCCCAGCCCCT |
| | | GCCCGGCCCGGCCGCCCTGCGGGGAGGGGGCGACGGCGGAGGCGCGCT |
| | | GTAGGGGACCCCGGGCCACCGCCCGCCCCTCCGCTCCCCTCCCCCCTCC |
| | | TCCCCCCCCTGGCGGGGCGGGGCCGGGGCTGCACGGGCCCTCTGCCCC |
| | | CTCGGCCGGCGCCCGGCGGGCGGCGCTGCTGCGGGACGGGGGCGGCCG |
| | | CAGTAACCGGGATTCGTTCTGGAACTTTCAGGTGTCGTTTCTCTTTGGCC |
| | | GGAAGAAAGAAGCTAAAGACCATCCGAAACGTAGTCGACTCGAGGCA |
| | | GGAAAATGGCAAGCGCCGGGGCACACACGGATGAGCGCTGCCGGCAG |
| | | CCTGCCTTCCTCCCCGGCCCGCCGCCGACAGAAGTTGAGAGCGGCAGC |
| | | GGCAGCGCCAGCGCCAAGGTGCAGAGGATGTGTGCTGCCAGGCAGATA |
| | | CTGCTGCTACTGCTGGCCTTCCTGGCCTATGCGTTGGATTCAGCTGCGG |
| | | CGTATGGCACGGCGGAGACCCTCTGCGGTGGGGAGCTGGTGGACACAC |
| | | TGCAGTTCGTCTGTGGGGACAGGGGCTTCTACTTCAGTAGACCAGTGGG |
| | | ACGAAATAACAGGAGGATCAACCGTGGCATTGTGGAGGAGTGCTGCTT |
| | | TCGGAGCTGTGACCTGGCTCTGCTGGAAACCTACTGTGCCAAGTCCGTC |
| | | AAGTCAGAGCGTGACCTCTCCGCCACCTCCCTCGCGGGCCTCCCAGCCC |
| | | TCAACAAGGAGAGCTTCCAGAAGCCATCTCATGCCAAGTACTCCAAGT |
| | | ACAACGTGTGGCAGAAGAAGAGCTCGCAGCGGCTGCAGCGGGAGGTG |
| | | CCAGGCATCCTGCGTGCCCGTCGGTACCGGTGGCAGGCGGAGGGGCTG |
| | | CAAGCAGCTGAGGAAGCCAGGGCGATGCATCGTCCCCTCATCTCCTTGC |
| | | CCAGTCAGCGGCCCCCAGCGCCGCGGGCATCCCCTGAAGCGACCGGCC |
| | | CCCAGGAATGATAAGATCGTATCGTTAACGCGGCCGCCACGTGCTCGA |
| | | GGAGTTGCCAGCCATCTGTTGTTTGCCCCTCCCCCGTGCCTTCCTTGACC |
| | | CTGGAAGGTGCCACTCCCACTGTCCTTTCCTAATAAAATGAGGAAATTG |
| | | CATCATAACTTCGTATAGCATACATTATACGAACGGTAGGATCCCCGGG |
| | | TACCGAGCTCGAATTCACTGGCCGTCGTTTTACAACGTCGTGACTGGGA |
| | | AAACCCTGGCGTTACCCAACTTAATCGCCTTGCAGCACATCCCCCTTTC |
| | | GCCAGCTGGCGTAATAGCGAAGAGGCCCGCACCGATCGCCCTTCCCAA |
| | | CAGTTGCGCAGCCTGAATGGCGAATGGCGCCTGATGCGGTATTTTCTCC |
| | | TTACGCATCTGTGCGGTATTTCACACCGCATATGGTGCACTCTCAGTAC |
| | | AATCTGCTCTGATGCCGCATAGTTAAGCCAGCCCCGACACCCGCCAACA |
| | | CCCGCTGACGCGCCCTGACGGGCTTGTCTGCTCCCGGCATCCGCTTACA |
| | | GACAAGCTGTGACCGTCTCCGGGAGCTGCATGTGTCAGAGGTTTTCACC |
| | | GTCATCACCGAAACGCGCGAGACGAAAGGGCCTCGTGATACGCCTATT |
| | | TTTATAGGTTAATGTCATGATAATAATGGTTTCTTAGACGTCAGGTGGC |
| | | ACTTTTCGGGGAAATGTGCGCGGAACCCCTATTTGTTTATTTTTCTAAAT |
| | | ACATTCAAATATGTATCCGCTCATGAGACAATAACCCTGATAAATGCTT |
| | | CAATAATATTGAAAAAGGAAGAGTATGAGTATTCAACATTTCCGTGTC |
| | | GCCCTTATTCCCTTTTTTGCGGCATTTTGCCTTCCTGTTTTTGCTCACCCA |
| | | GAAACGCTGGTGAAAGTAAAAGATGCTGAAGATCAGTTGGGTGCACGA |
| | | GTGGGTTACATCGAACTGGATCTCAACAGCGGTAAGATCCTTGAGAGTT |
| | | TTCGCCCCGAAGAACGTTTTCCAATGATGAGCACTTTTAAAGTTCTGCT |
| | | ATGTGGCGCGGTATTATCCCGTATTGACGCCGGGCAAGAGCAACTCGG |
| | | TCGCCGCATACACTATTCTCAGAATGACTTGGTTGAGTACTCACCAGTC |
| | | ACAGAAAAGCATCTTACGGATGGCATGACAGTAAGAGAATTATGCAGT |
| | | GCTGCCATAACCATGAGTGATAACACTGCGGCCAACTTACTTCTGACAA |
| | | CGATCGGAGGACCGAAGGAGCTAACCGCTTTTTTGCACAACATGGGGG |
| | | ATCATGTAACTCGCCTTGATCGTTGGGAACCGGAGCTGAATGAAGCCAT |
| | | ACCAAACGACGAGCGTGACACCACGATGCCTGTAGCAATGGCAACAAC |
| | | GTTGCGCAAACTATTAACTGGCGAACTACTTACTCTAGCTTCCCGGCAA |
| | | CAATTAATAGACTGGATGGAGGCGGATAAAGTTGCAGGACCACTTCTG |
| | | CGCTCGGCCCTTCCGGCTGGCTGGTTTATTGCTGATAAATCTGGAGCCG |
| | | GTGAGCGTGGCTCTCGCGGTATCATTGCAGCACTGGGGCCAGATGGTA |
| | | AGCCCTCCCGTATCGTAGTTATCTACACGACGGGGAGTCAGGCAACTAT |
| | | GGATGAACGAAATAGACAGATCGCTGAGATAGGTGCCTCACTGATTAA |
| | | GCATTGGTAACTGTCAGACCAAGTTTACTCATATATACTTTAGATTGAT |
| | | TTAAAACTTCATTTTTAATTTAAAAGGATCTAGGTGAAGATCCTTTTTG |
| | | ATAATCTCATGACCAAAATCCCTTAACGTGAGTTTTCGTTCCACTGAGC |
| | | GTCAGACCCCGTAGAAAAGATCAAAGGATCTTCTTGAGATCCTTTTTTT |
| | | CTGCGCGTAATCTGCTGCTTGCAAACAAAAAAACCACCGCTACCAGCG |
| | | GTGGTTTGTTTGCCGGATCAAGAGCTACCAACTCTTTTTCCGAAGGTAA |
| | | CTGGCTTCAGCAGAGCGCAGATACCAAATACTGTTCTTCTAGTGTAGCC |
| | | GTAGTTAGGCCACCACTTCAAGAACTCTGTAGCACCGCCTACATACCTC |
| | | GCTCTGCTAATCCTGTTACCAGTGGCTGCTGCCAGTGGCGATAAGTCGT |
| | | GTCTTACCGGGTTGGACTCAAGACGATAGTTACCGGATAAGGCGCAGC |
| | | GGTCGGGCTGAACGGGGGGTTCGTGCACACAGCCCAGCTTGGAGCGAA |
| | | CGACCTACACCGAACTGAGATACCTACAGCGTGAGCTATGAGAAAGCG |
| | | CCACGCTTCCCGAAGGGAGAAAGGCGGACAGGTATCCGGTAAGCGGCA |
| | | GGGTCGGAACAGGAGAGCGCACGAGGGAGCTTCCAGGGGGAAACGCC |
| | | TGGTATCTTTATAGTCCTGTCGGGTTTCGCCACCTCTGACTTGAGCGTCG |
| | | ATTTTTGTGATGCTCGTCAGGGGGGCGGAGCCTATGGAAAAACGCCAG |
| | | CAACGCGGCCTTTTTACGGTTCCTGGCCTTTTGCTGGCCTTTTGCTCACA |
| | | TGTTCTTTCCTGCGTTATCCCCTGATTCTGTGGATAACCGTATTACCGCC |
| | | TTTGAGTGAGCTGATACCGCTCGCCGCAGCCGAACGACCGAGCGCAGC |
| | | GAGTCAGTGAGCGAGGAAGCGGAAGAGCGCCCAATACGCAAACCGCC |
| | | TCTCCCCGCGCGTTGGCCGATTCATTAATGCAGC |
| 98 | pFS2-ggEF1- | TGGCACGACAGGTTTCCCGACTGGAAAGCGGGCAGTGAGCGCAACGCA |
| | ggSDC4- | ATTAATGTGAGTTAGCTCACTCATTAGGCACCCCAGGCTTTACACTTTA |
| | mmPGK-Neo | TGCTTCCGGCTCGTATGTTGTGTGGAATTGTGAGCGGATAACAATTTCA |
| | (Promoters: | CACAGGAAACAGCTATGACCATGATTACGCCAAGCTTAAGCTTGCATG |
| | rodentia PGK | CCTGCAGGTCGACCTGCAGCGATCGAACTTGTTTATTGCAGCTTATAAT |
| | (mPGK) and | GGTTACAAATAAAGCAATAGCATCACAAATTTCACAAATAAAGCATTT |
| | gallus gallus | TTTTCACTGCATTCTAGTTGTGGTTTGTCCAAACTCATCAATGTATCTTA |
| | EF1 (ggEF1)) | TCATGTCTGGATCCCTTCTGAGGGCAGAGGAAGTCTTAATTCAGAAGAA |
| | | CTCGTCAAGAAGGCGATAGAAGGCGATGCGCTGCGAATCGGGAGCGGC |
| | | GATACCGTAAAGCACGAGGAAGCGGTCAGCCCATTCGCCGCCAAGCTC |
| | | TTCAGCAATATCACGGGTAGCCAACGCTATGTCCTGATAGCGGTCCGCC |
| | | ACACCCAGCCGGCCACAGTCGATGAATCCAGAAAAGCGGCCATTTTCC |
| | | ACCATGATATTCGGCAAGCAGGCATCGCCATGGGTCACGACGAGATCC |
| | | TCGCCGTCGGGCATGCTCGCCTTGAGCCTGGCGAACAGTTCGGCTGGCG |
| | | CGAGCCCCTGATGCTCTTCGTCCAGATCATCCTGATCGACAAGACCGGC |
| | | TTCCATCCGAGTACGTGCTCGCTCGATGCGATGTTTCGCTTGGTGGTCG |
| | | AATGGGCAGGTAGCCGGATCAAGCGTATGCAGCCGCCGCATTGCATCA |
| | | GCCATGATGGATACTTTCTCGGCAGGAGCAAGGTGAGATGACAGGAGA |
| | | TCCTGCCCCGGCACTTCGCCCAATAGCAGCCAGTCCCTTCCCGCTTCAG |
| | | TGACAACGTCGAGCACAGCTGCGCAAGGAACGCCCGTCGTGGCCAGCC |
| | | ACGATAGCCGCGCTGCCTCGTCTTGCAGTTCATTCAGGGCACCGGACAG |
| | | GTCGGTCTTGACAAAAAGAACCGGGCGCCCCTGCGCTGACAGCCGGAA |
| | | CACGGCGGCATCAGAGCAGCCGATTGTCTGTTGTGCCCAGTCATAGCCG |
| | | AATAGCCTCTCCACCCAAGCGGCCGGAGAACCTGCGTGCAATCCATCTT |
| | | GTTCAATCATGCCCATGGTGGCGGCACGTGAGGTAGGTCGAAAGGCCC |
| | | GGAGATGAGGAAGAGGAGAACAGCGCGGCAGACGTGCGCTTTTGAAG |
| | | CGTGCAGAATGCCGGGCCTCCGGAGGACCTTCGGGCGCCCGCCCCGCC |
| | | CCTGAGCCCGCCCCTGAGCCCGCCCCCGGACCCACCCTTCCCAGCTGCT |
| | | GAGCCCAGAAAGCGAAGGAGCAAAGCTGCTATTGGCCGCTGCCCCAAA |
| | | GGCCTACCCGCTTCCATTGCTCAGCGGTGCTGTCCATCTGCACGAGACT |
| | | AGTGAGACGTGCTACTTCCATTTGTCACGTCCTGCACGACGCGAGCTGC |
| | | GGGGCGGGGGGGAACTTCCTGACTAGGGGAGGAGTAGAAGGTGGCGC |
| | | GAAGGGGCCACCAAAGAACGGAGCCGGTTGGCGCTACCGGTGGATGTG |
| | | GAATGTGTGCGAGGCCAGAGGCCACTTGTGTAGCGCCAAGTGCCAGCG |
| | | GGGCTGCTAAAGCGCATGCTCCAGACTGCCTTGGGAAAAGCGCCTCCC |
| | | CTACCCGGTAGAATTGGTGGCGATGCCGAGCTCGGTACCAAGCTTAAG |
| | | TTTAAACGCTAGCAGTAGAATTCGGATCCTACCGTTCGTATAGCATACA |
| | | TTATACGAAGTTATGTCGACGATGTAGGTCACGGTCTCGAAGCCGCGGT |
| | | GCGGGTGCCAGGGCGTGCCCTTGGGCTCCCCGGGCGCGTACTCCACCTC |
| | | ACCCATCTGGTCCATCATGATGAACGGTCGAGGTGGCGGTAGTTGATCC |
| | | CGGCGAACGCGCGGCGCACCGGGAAGCCCTCGCCCTCGAAACCGCTGG |
| | | GCGCGGTGGTCACGGTGAGCACGGGACGTGCGACGGCGTCGGCGGGTG |
| | | CGGATACGCGGGGCAGCGTCAGCGGGTTCTCGACGGTCACGGCGGGCA |
| | | TGTCGACGGCGCGCCGATGCCATGAGTACACCCTGCTGGAATCACGAG |
| | | GGCTTTCACTGCTGCTGCCTCGTGGCTGCTCGCCGCCCCGGGTTCAGAA |
| | | GCGAAAGGCTGGGGCTGCCAGCACCTTCGGTGGCTTTCGTCAGCACCC |
| | | GTCAGCCGGGAACATGTGGCGTGGGGGCACTGCGCGCAGTTTGGAGCT |
| | | CTCAAGCAGAAGCCACGAATCCTTGCCGCTGAAGCGATTCTCTCTGGCC |
| | | GCATCTGAAACGCCAGGCCAGAGGAGGTCTGTGCAAGTAACTACTCCC |
| | | GGAGCTCGCTGACCGCCTCCCCTCGGGCCGCAGCCGCGGGTCAGAGGG |
| | | GCAGAGCTCGGGGACTCTACGGCTGCCGTGCTGCGCGGGAGGCGGCGG |
| | | AGCGGCAACGCCGTCCCGCCCTGCAGTGCTCCGGGCGCTGCCGTCCGG |
| | | CCTGGGGGCGGCGAGCCCCTCCCGGCAGTCGCTCGTGACTCCCGCCCG |
| | | ATCCGGCCGCCGCGGGGCTCTGCTCCTCAGCCCCGCCCCTCCCCGCCGC |
| | | TCCTCAGCGGAGCGCCGCGTGCCGCGCTGCCGGGCGGGGCGTTGCGCG |
| | | GGGGCCGCCGGGAGCGGTGACGCGTCGCCGAGGCCCCGCCCCGTCGCA |
| | | GAGTATATAAGGGGCGGCGCTCACGCCGTCGTTCTTTTCCGCTCCGGGT |
| | | TTGCCGACGCCGTGCGGGTGAGGCGCGTGCGGCGGGGCAGGCCGGGCC |
| | | CGGGGGTGGGGGGTGCGGGGTGCGGGGATGCTCGGGGGGAGCCTCGTT |
| | | GCACCGTGAGCGCGGGGCCGGGGCGCGGCCGCGGAGAGGCGCGTGGG |
| | | AACGGCGGAGTGGGGAGGGGGAAGCGAGATGAGGGACGGGTGGGAGA |
| | | GAGCGGGGCGAGCGCCGGGGTAAAGCCGACGTGTCCCGCCTGAGGACC |
| | | CACGCGGGGCAGCGCGGCGATGAGCACGCGGGGCCCCACCGCCGCCTT |
| | | GGCTTCGCGCGGAGCCGCCCGGCGCTGGGGCGCGGCGTACGGTGCTCG |
| | | GCCTCGGGCGGGTGGCGGCCGGGGACCCCGGGGCCGCGCCGTCGGTGC |
| | | CGCGGTGGGGGAGGGGAGGCGAGGCCGGAGCACGAGAAAAAAAATGG |
| | | CCGCCCGCTCTTCCTGCCGCCGCCAGCTCACAAAATGGAGGACGGCGG |
| | | CCCGGGCGGGGTGAGTCACACACAAAGGAGAGGGGCGGCCCCGCCCTC |
| | | CGCGCCGCCCGTGGCCGCGCCGCCGCCCCGCGCCCCCACCCCAGCCCCT |
| | | GCCCGGCCCGGCCGCCCTGCGGGGAGGGGGCGACGGCGGAGGCGCGCT |
| | | GTAGGGGACCCCGGGCCACCGCCCGCCCCTCCGCTCCCCTCCCCCCTCC |
| | | TCCCCCCCCTGGCGGGGGGGGCCGGGGCTGCACGGGCCCTCTGCCCC |
| | | CTCGGCCGGCGCCCGGCGGGCGGCGCTGCTGCGGGACGGGGGCGGCCG |
| | | CAGTAACCGGGATTCGTTCTGGAACTTTCAGGTGTCGTTTCTCTTTGGCC |
| | | GGAAGAAAGAAGCTAAAGACCATCCGAAACGTAGTCGACTCGAGGCA |
| | | GGAAAATGCCGCTGCCCCGCGCCGCGTTCCTGCTCGGCCTCCTGCTGGC |
| | | CGCTGCCGCCGCCGAGTCGGTGAGAGAAACAGAGACCATGGATGCCCG |
| | | ATGGCTTGACAACGTGGGCTCTGGAGACCTGCCAGATGATGAAGACAT |
| | | TGGTGAATTCACACCTCACTTAACTTCTGACGAGTTTGATATAGATGAC |
| | | ACATCTGGCTCCGGAGACTACTCAGATTATGATGATGCCATATACCTGA |
| | | CCACTGTGGATACTCCTGCAATATCTGACAACTATATCCCTGGAGATAC |
| | | AGAGAGAAAGATGGAAGGTGAGAAGAAAAACACCATGCTGGACAATG |
| | | AAATCATTCCAGACAAAGCTTCACCTGTTGAAGCAAACCTGTCCAACA |
| | | AGATCTCCATGGCAAGCACAGCCAACAGCAGCATCTTTGAAAGAACAG |
| | | AAGTTCTTACAGCTCTCATTGCAGGAGGAGCAGTTGGCCTCCTGTTTGC |
| | | TGTCTTCCTGATCCTCCTCTTAGTCTATCGCATGAAGAAAAAGGACGAG |
| | | GGCAGCTACGACCTTGGGAAGAAACCCATCTACAAGAAAGCCCCTACA |
| | | AATGAGTTCTACGCTTAATAAGATCGTATCGTTAACGCGGCCGCCACGT |
| | | GCTCGAGGAGTTGCCAGCCATCTGTTGTTTGCCCCTCCCCCGTGCCTTCC |
| | | TTGACCCTGGAAGGTGCCACTCCCACTGTCCTTTCCTAATAAAATGAGG |
| | | AAATTGCATCATAACTTCGTATAGCATACATTATACGAACGGTAGGATC |
| | | CCCGGGTACCGAGCTCGAATTCACTGGCCGTCGTTTTACAACGTCGTGA |
| | | CTGGGAAAACCCTGGCGTTACCCAACTTAATCGCCTTGCAGCACATCCC |
| | | CCTTTCGCCAGCTGGCGTAATAGCGAAGAGGCCCGCACCGATCGCCCTT |
| | | CCCAACAGTTGCGCAGCCTGAATGGCGAATGGCGCCTGATGCGGTATTT |
| | | TCTCCTTACGCATCTGTGCGGTATTTCACACCGCATATGGTGCACTCTCA |
| | | GTACAATCTGCTCTGATGCCGCATAGTTAAGCCAGCCCCGACACCCGCC |
| | | AACACCCGCTGACGCGCCCTGACGGGCTTGTCTGCTCCCGGCATCCGCT |
| | | TACAGACAAGCTGTGACCGTCTCCGGGAGCTGCATGTGTCAGAGGTTTT |
| | | CACCGTCATCACCGAAACGCGCGAGACGAAAGGGCCTCGTGATACGCC |
| | | TATTTTTATAGGTTAATGTCATGATAATAATGGTTTCTTAGACGTCAGGT |
| | | GGCACTTTTCGGGGAAATGTGCGCGGAACCCCTATTTGTTTATTTTTCTA |
| | | AATACATTCAAATATGTATCCGCTCATGAGACAATAACCCTGATAAATG |
| | | CTTCAATAATATTGAAAAAGGAAGAGTATGAGTATTCAACATTTCCGTG |
| | | TCGCCCTTATTCCCTTTTTTGCGGCATTTTGCCTTCCTGTTTTTGCTCACC |
| | | CAGAAACGCTGGTGAAAGTAAAAGATGCTGAAGATCAGTTGGGTGCAC |
| | | GAGTGGGTTACATCGAACTGGATCTCAACAGCGGTAAGATCCTTGAGA |
| | | GTTTTCGCCCCGAAGAACGTTTTCCAATGATGAGCACTTTTAAAGTTCT |
| | | GCTATGTGGCGCGGTATTATCCCGTATTGACGCCGGGCAAGAGCAACTC |
| | | GGTCGCCGCATACACTATTCTCAGAATGACTTGGTTGAGTACTCACCAG |
| | | TCACAGAAAAGCATCTTACGGATGGCATGACAGTAAGAGAATTATGCA |
| | | GTGCTGCCATAACCATGAGTGATAACACTGCGGCCAACTTACTTCTGAC |
| | | AACGATCGGAGGACCGAAGGAGCTAACCGCTTTTTTGCACAACATGGG |
| | | GGATCATGTAACTCGCCTTGATCGTTGGGAACCGGAGCTGAATGAAGC |
| | | CATACCAAACGACGAGCGTGACACCACGATGCCTGTAGCAATGGCAAC |
| | | AACGTTGCGCAAACTATTAACTGGCGAACTACTTACTCTAGCTTCCCGG |
| | | CAACAATTAATAGACTGGATGGAGGCGGATAAAGTTGCAGGACCACTT |
| | | CTGCGCTCGGCCCTTCCGGCTGGCTGGTTTATTGCTGATAAATCTGGAG |
| | | CCGGTGAGCGTGGCTCTCGCGGTATCATTGCAGCACTGGGGCCAGATG |
| | | GTAAGCCCTCCCGTATCGTAGTTATCTACACGACGGGGAGTCAGGCAA |
| | | CTATGGATGAACGAAATAGACAGATCGCTGAGATAGGTGCCTCACTGA |
| | | TTAAGCATTGGTAACTGTCAGACCAAGTTTACTCATATATACTTTAGAT |
| | | TGATTTAAAACTTCATTTTTAATTTAAAAGGATCTAGGTGAAGATCCTT |
| | | TTTGATAATCTCATGACCAAAATCCCTTAACGTGAGTTTTCGTTCCACTG |
| | | AGCGTCAGACCCCGTAGAAAAGATCAAAGGATCTTCTTGAGATCCTTTT |
| | | TTTCTGCGCGTAATCTGCTGCTTGCAAACAAAAAAACCACCGCTACCAG |
| | | CGGTGGTTTGTTTGCCGGATCAAGAGCTACCAACTCTTTTTCCGAAGGT |
| | | AACTGGCTTCAGCAGAGCGCAGATACCAAATACTGTTCTTCTAGTGTAG |
| | | CCGTAGTTAGGCCACCACTTCAAGAACTCTGTAGCACCGCCTACATACC |
| | | TCGCTCTGCTAATCCTGTTACCAGTGGCTGCTGCCAGTGGCGATAAGTC |
| | | GTGTCTTACCGGGTTGGACTCAAGACGATAGTTACCGGATAAGGCGCA |
| | | GCGGTCGGGCTGAACGGGGGGTTCGTGCACACAGCCCAGCTTGGAGCG |
| | | AACGACCTACACCGAACTGAGATACCTACAGCGTGAGCTATGAGAAAG |
| | | CGCCACGCTTCCCGAAGGGAGAAAGGCGGACAGGTATCCGGTAAGCGG |
| | | CAGGGTCGGAACAGGAGAGCGCACGAGGGAGCTTCCAGGGGGAAACG |
| | | CCTGGTATCTTTATAGTCCTGTCGGGTTTCGCCACCTCTGACTTGAGCGT |
| | | CGATTTTTGTGATGCTCGTCAGGGGGGCGGAGCCTATGGAAAAACGCC |
| | | AGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCG |
| | | CCTTTGAGTGAGCTGATACCGCTCGCCGCAGCCGAACGACCGAGCGCA |
| | | GCGAGTCAGTGAGCGAGGAAGCGGAAGAGCGCCCAATACGCAAACCG |
| | | CCTCTCCCCGCGCGTTGGCCGATTCATTAATGCAGC |
| 99 | pFS2- | TGGCACGACAGGTTTCCCGACTGGAAAGCGGGCAGTGAGCGCAACGCA |
| | ggGAPDH_1k | ATTAATGTGAGTTAGCTCACTCATTAGGCACCCCAGGCTTTACACTTTA |
| | b-FAP- | TGCTTCCGGCTCGTATGTTGTGTGGAATTGTGAGCGGATAACAATTTCA |
| | mmPGK-Neo | CACAGGAAACAGCTATGACCATGATTACGCCAAGCTTAAGCTTGCATG |
| | (Promoters: | CCTGCAGGTCGACCTGCAGCGATCGAACTTGTTTATTGCAGCTTATAAT |
| | rodentia PGK | GGTTACAAATAAAGCAATAGCATCACAAATTTCACAAATAAAGCATTT |
| | (mPGK) and | TTTTCACTGCATTCTAGTTGTGGTTTGTCCAAACTCATCAATGTATCTTA |
| | gallus gallus | TCATGTCTGGATCCCTTCTGAGGGCAGAGGAAGTCTTAATTCAGAAGAA |
| | GAPDH-1kb | CTCGTCAAGAAGGCGATAGAAGGCGATGCGCTGCGAATCGGGAGCGGC |
| | (ggGAPDH_1k)) | GATACCGTAAAGCACGAGGAAGCGGTCAGCCCATTCGCCGCCAAGCTC |
| | | TTCAGCAATATCACGGGTAGCCAACGCTATGTCCTGATAGCGGTCCGCC |
| | | ACACCCAGCCGGCCACAGTCGATGAATCCAGAAAAGCGGCCATTTTCC |
| | | ACCATGATATTCGGCAAGCAGGCATCGCCATGGGTCACGACGAGATCC |
| | | TCGCCGTCGGGCATGCTCGCCTTGAGCCTGGCGAACAGTTCGGCTGGCG |
| | | CGAGCCCCTGATGCTCTTCGTCCAGATCATCCTGATCGACAAGACCGGC |
| | | TTCCATCCGAGTACGTGCTCGCTCGATGCGATGTTTCGCTTGGTGGTCG |
| | | AATGGGCAGGTAGCCGGATCAAGCGTATGCAGCCGCCGCATTGCATCA |
| | | GCCATGATGGATACTTTCTCGGCAGGAGCAAGGTGAGATGACAGGAGA |
| | | TCCTGCCCCGGCACTTCGCCCAATAGCAGCCAGTCCCTTCCCGCTTCAG |
| | | TGACAACGTCGAGCACAGCTGCGCAAGGAACGCCCGTCGTGGCCAGCC |
| | | ACGATAGCCGCGCTGCCTCGTCTTGCAGTTCATTCAGGGCACCGGACAG |
| | | GTCGGTCTTGACAAAAAGAACCGGGCGCCCCTGCGCTGACAGCCGGAA |
| | | CACGGCGGCATCAGAGCAGCCGATTGTCTGTTGTGCCCAGTCATAGCCG |
| | | AATAGCCTCTCCACCCAAGCGGCCGGAGAACCTGCGTGCAATCCATCTT |
| | | GTTCAATCATGCCCATGGTGGCGGCACGTGAGGTAGGTCGAAAGGCCC |
| | | GGAGATGAGGAAGAGGAGAACAGCGCGGCAGACGTGCGCTTTTGAAG |
| | | CGTGCAGAATGCCGGGCCTCCGGAGGACCTTCGGGCGCCCGCCCCGCC |
| | | CCTGAGCCCGCCCCTGAGCCCGCCCCCGGACCCACCCTTCCCAGCTGCT |
| | | GAGCCCAGAAAGCGAAGGAGCAAAGCTGCTATTGGCCGCTGCCCCAAA |
| | | GGCCTACCCGCTTCCATTGCTCAGCGGTGCTGTCCATCTGCACGAGACT |
| | | AGTGAGACGTGCTACTTCCATTTGTCACGTCCTGCACGACGCGAGCTGC |
| | | GGGGCGGGGGGGAACTTCCTGACTAGGGGAGGAGTAGAAGGTGGCGC |
| | | GAAGGGGCCACCAAAGAACGGAGCCGGTTGGCGCTACCGGTGGATGTG |
| | | GAATGTGTGCGAGGCCAGAGGCCACTTGTGTAGCGCCAAGTGCCAGCG |
| | | GGGCTGCTAAAGCGCATGCTCCAGACTGCCTTGGGAAAAGCGCCTCCC |
| | | CTACCCGGTAGAATTGGTGGCGATGCCGAGCTCGGTACCAAGCTTAAG |
| | | TTTAAACGCTAGCAGTAGAATTCGGATCCTACCGTTCGTATAGCATACA |
| | | TTATACGAAGTTATGTCGACGATGTAGGTCACGGTCTCGAAGCCGCGGT |
| | | GCGGGTGCCAGGGCGTGCCCTTGGGCTCCCCGGGCGCGTACTCCACCTC |
| | | ACCCATCTGGTCCATCATGATGAACGGTCGAGGTGGCGGTAGTTGATCC |
| | | CGGCGAACGCGCGGCGCACCGGGAAGCCCTCGCCCTCGAAACCGCTGG |
| | | GCGCGGTGGTCACGGTGAGCACGGGACGTGCGACGGCGTCGGCGGGTG |
| | | CGGATACGCGGGGCAGCGTCAGCGGGTTCTCGACGGTCACGGCGGGCA |
| | | TGTCGACGGCGCGCCGATGATGCTGCAGTTCCTCATTAGCACGGTCCTC |
| | | ACGCAGCACCGCACGGCCTGCTGGGCCGGGCCCGGCCGGGAGCCTCAG |
| | | AGCGGGGCCGTTGGGCGGCCGTGCACCCCGGTATCGCGGTGCGGGGAC |
| | | GGATATGGGACCCAGCGCTCCCCAGCAGCTCGGGGCTGCGCGGCCAAC |
| | | ACGCGCAAGGGCTGAGGACGGGGAGCGGGCAGGCTGCAGCCTCGCATC |
| | | GCGCTCTTTGTCCCGCCCGCGGCCAGACGCCGTTCTCTGGGTACCCGCA |
| | | GGAGCGCAATCCGCACCCACTGTACCGCATCGCGTGATGCAGCCGCAT |
| | | CGCCCCGGCCTCGTCCGAGGACGCCACGGCCCCACGTGGGGCTTTGTCT |
| | | GCACGCAGCTCCGCGCCCCCCGGCCCCGGGGCACCCCTCGCCGCCCCC |
| | | ACCCCGCACGGGCACCGTGGTACGGCAGCAACCCCCTAAGCCCACCCT |
| | | GAGCCCAACGGCCGCCCTTTGTTGCGAGGGGGGAGGGGGAAGGAGAG |
| | | GAGAGGGGGGGCCGTTTCGCCCGCACCGTGCGCGCACGTAGCCCCGAG |
| | | CCCGCCCCCGCGCTGCCATTCGGCGCTGGCCCGGCGGGGCGGGGCGCG |
| | | GGGCGCTGCCTCCGTGTCTATAAAGGGCGGCGGCGCGGCACTGCGCCA |
| | | CCTTCTCACTGCGCGCTGGGGCCGTTGACGTGCAGCAGGTAACGGGCG |
| | | GGGGGACGTGGGGGCGGAGAAGCGTACCCCCCACCCACCCGCCTGTCT |
| | | GCCTGCGCCCCCCTGTAGGGGTTGGGGCTGTAGGACCGCGAGGAGTGC |
| | | GGCGGGGGAAGGGGGGAGGGGGTCTCCGCGCAGGACCGCGTGGTGCC |
| | | CGGGGCACAAAGGCGGGGGGGGGGGGCGCGAGATGAAGGGGGTGGGG |
| | | GTTGGGGGAGAGAGGGAGAACTGCGGGGGAGAGAGCTCGATGGGGAT |
| | | GAAGGAAGGGATGGCGGGTCGGGGGGCGGTGCCCAGAGCTCCGCAGC |
| | | GCGCGCGGTGGGGGGACGTTACTGGGCCGGCGGTCTGCGTGCGGCACC |
| | | GCACGTCTCACTGCCACGTAGTCGACTCGAGGCAGGAAAATGAAGACC |
| | | CGGCTAAAAGTACTATTTGGCGTGGTTGTATGTGTTTTGCTTTCCTTATT |
| | | ACTTATGTGTATTGTACTGCTTCCGTCCAAAGTTGTCACCACAGTTGAT |
| | | GGTCCTAGAGCTCTTACACTGGATGATTACTTGAATGGAAATTTTCAAT |
| | | ATAAAACATATTTTCCATACTGGGTGTCAGATAGTGAATATCTTCACCA |
| | | GAATCAAGAAGATGATATTATTCTTTTCAATGTTGACATGAACTACCTA |
| | | ACTACCATCATGACCAACAGCACAATGAAACAAGTTAATGCTTCAAAT |
| | | TATGTGATGTCATCAGACAAATACTTCATAGCTCTGGAGAGCAATTATT |
| | | CAAAGCTGTGGAGATATTCTTATACTGCATCATACCACATTTATGATCT |
| | | CATATATGGTGGTTTTGTAACAGAAAATCAGCTTCCACATAAAATTCAG |
| | | TATATATCCTGGTCACCCGTTGGACACAAATTGGCGTATGTTTACCAAA |
| | | ATAATATCTATTTGAAACAAAGTCCAAGAGAGGCACCAATTAAACTAA |
| | | CTAGTGATGGAAAAGAAAATGAAATATTTAATGGGATACCAGATTGGG |
| | | TCTATGAAGAGGAAATGCTAGCAACTAAATATGCATTGTGGTGGTCTCC |
| | | AAGTGGAAAATACTTAGCATATGTACAATTTAATGATTCTGATATACCA |
| | | GTAATTGAATATTCGTATTTTGGCGAGGACCAATACCCAAGAAAAATA |
| | | ATTATCCCATACCCCAAGGCTGGTGCTAAAAATCCTACTGTTAAAGTGT |
| | | TCATTGTAGACACTACTAACATTGAAGCATTTGGTCCTAAAGAGGTGCC |
| | | AGTTCCTGCAGTGATTGCATCAAGTGATCATTATTTTACTTGGCTTACTT |
| | | GGGTAACAGATAGTCGAGTTGGTGTGCAATGGCTAAAGAGAATCCAAA |
| | | ATTTTTCAGTCTTAGCTATTTGTGACTTCAAAGAAAATTCAAATACTTG |
| | | GGACTGTCCAGAGAATCAACAGCACATCGAGGAGAGTCAAACAGGATG |
| | | GGCAGGCGGATTCTTTGTTTCTGCACCATATTTTACCTCAGACGGCAGT |
| | | TCGTACTACAAAATTTTTAGTGACAAAAATGGTTATAAGCATATACATT |
| | | ACATCAATGGTTCTGTGGAGAATGCAATCCAAATTACAAGTGGAGAAT |
| | | GGGAGGCAATATATATTTTTAGAGTAACAAATGATGCAATTTTCTACTC |
| | | AAGCAATGAATTTGAAGGCTATCCAGGAAGAAGAAATATTTACAAAAT |
| | | TAGTATTGGAAGTAAGCCTATCAGAAAACTATGCATTACTTGCAATTTA |
| | | CGGAAAGAGAGATGCCAGTATTATACAGCGAGGTTCAGTGAACGCTCT |
| | | AAGTATTATGCCTTGATCTGTTATGGTCCCGGGATTCCTATTTCTACTCT |
| | | TTTTGAGAATGAGAGTGATAGAGAGCTCAGAATATTAGAGGACAATCA |
| | | AGAATTGCAGTCTGCTTTGCAAGAGATCATACTGCCAAAAGAAGAAAT |
| | | TAATAAACTTGAAGTGGATGGTATAACTTTGTGGTACAAAATGCTTATA |
| | | CCCCCACAATTTGATAGATCCAAGAAGTACCCACTGCTTATTCAAGTGT |
| | | ATGGAGGACCATGCAGTCAGAATGTAAAACACACATTTAGCATTAGCT |
| | | GGATAACGTATCTTGCAAGCAAAGAGGGAATTATTGTTGCTCTAGTAG |
| | | ATGGCAGAGGAACAGCTTATCAAGGTGACAAGATTTTGCATGCAGTTT |
| | | ATCGAAGACTAGGAGTCTATGAAGTTGAGGACCAAATTTCAGCTGTGA |
| | | AGAAATTTATAGAAATGGGTTTTATTGATGAGAAACGAATAGCAATAT |
| | | GGGGCTGGTCCTATGGTGGATATGTAACTTCTCTGGCACTTGGATCTGG |
| | | CAGTGGAGTATTTAAATGTGGAATAGCTGTGGCTCCTGTTTCCAGTTGG |
| | | GAATATTACGCATCTATCTACACAGAACGATTTATGGGTCTTCCTGTAG |
| | | AGTCCGATAATCTTGAGCACTATAAGAATTCAACTGTGATGGCAAGAG |
| | | CAAAGAATTTCCAAAACGTTGAGTATCTTCTCATTCATGGAACAGCAGA |
| | | TGATAACGTACATTTTCAGAACTCAGCACAAATTGCAAAAGCTCTGGTT |
| | | AATGCACAGGTGGATTTCCAGGCAATGTGGTACACTGATCAGAACCAT |
| | | GGAATTCCAGGACTTTCCAGCAAACATCTCTACACACATATGACCCACT |
| | | TCCTCAAACAATGTTTTTCTTTGTCAGAATAGTAAGATCGTATCGTTAA |
| | | CGCGGCCGCCACGTGCTCGAGGAGTTGCCAGCCATCTGTTGTTTGCCCC |
| | | TCCCCCGTGCCTTCCTTGACCCTGGAAGGTGCCACTCCCACTGTCCTTTC |
| | | CTAATAAAATGAGGAAATTGCATCATAACTTCGTATAGCATACATTATA |
| | | CGAACGGTAGGATCCCCGGGTACCGAGCTCGAATTCACTGGCCGTCGTT |
| | | TTACAACGTCGTGACTGGGAAAACCCTGGCGTTACCCAACTTAATCGCC |
| | | TTGCAGCACATCCCCCTTTCGCCAGCTGGCGTAATAGCGAAGAGGCCCG |
| | | CACCGATCGCCCTTCCCAACAGTTGCGCAGCCTGAATGGCGAATGGCG |
| | | CCTGATGCGGTATTTTCTCCTTACGCATCTGTGCGGTATTTCACACCGCA |
| | | TATGGTGCACTCTCAGTACAATCTGCTCTGATGCCGCATAGTTAAGCCA |
| | | GCCCCGACACCCGCCAACACCCGCTGACGCGCCCTGACGGGCTTGTCTG |
| | | CTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGG |
| | | GCCTCGTGATACGCCTATTTTTATAGGTTAATGTCATGATAATAATGGT |
| | | TTCTTAGACGTCAGGTGGCACTTTTCGGGGAAATGTGCGCGGAACCCCT |
| | | ATTTGTTTATTTTTCTAAATACATTCAAATATGTATCCGCTCATGAGACA |
| | | ATAACCCTGATAAATGCTTCAATAATATTGAAAAAGGAAGAGTATGAG |
| | | TATTCAACATTTCCGTGTCGCCCTTATTCCCTTTTTTGCGGCATTTTGCCT |
| | | TCCTGTTTTTGCTCACCCAGAAACGCTGGTGAAAGTAAAAGATGCTGAA |
| | | GATCAGTTGGGTGCACGAGTGGGTTACATCGAACTGGATCTCAACAGC |
| | | GGTAAGATCCTTGAGAGTTTTCGCCCCGAAGAACGTTTTCCAATGATGA |
| | | GCACTTTTAAAGTTCTGCTATGTGGCGCGGTATTATCCCGTATTGACGC |
| | | CGGGCAAGAGCAACTCGGTCGCCGCATACACTATTCTCAGAATGACTT |
| | | GGTTGAGTACTCACCAGTCACAGAAAAGCATCTTACGGATGGCATGAC |
| | | AGTAAGAGAATTATGCAGTGCTGCCATAACCATGAGTGATAACACTGC |
| | | GGCCAACTTACTTCTGACAACGATCGGAGGACCGAAGGAGCTAACCGC |
| | | TTTTTTGCACAACATGGGGGATCATGTAACTCGCCTTGATCGTTGGGAA |
| | | CCGGAGCTGAATGAAGCCATACCAAACGACGAGCGTGACACCACGATG |
| | | CCTGTAGCAATGGCAACAACGTTGCGCAAACTATTAACTGGCGAACTA |
| | | CTTACTCTAGCTTCCCGGCAACAATTAATAGACTGGATGGAGGCGGATA |
| | | AAGTTGCAGGACCACTTCTGCGCTCGGCCCTTCCGGCTGGCTGGTTTAT |
| | | TGCTGATAAATCTGGAGCCGGTGAGCGTGGCTCTCGCGGTATCATTGCA |
| | | GCACTGGGGCCAGATGGTAAGCCCTCCCGTATCGTAGTTATCTACACGA |
| | | CGGGGAGTCAGGCAACTATGGATGAACGAAATAGACAGATCGCTGAGA |
| | | TAGGTGCCTCACTGATTAAGCATTGGTAACTGTCAGACCAAGTTTACTC |
| | | ATATATACTTTAGATTGATTTAAAACTTCATTTTTAATTTAAAAGGATCT |
| | | AGGTGAAGATCCTTTTTGATAATCTCATGACCAAAATCCCTTAACGTGA |
| | | GTTTTCGTTCCACTGAGCGTCAGACCCCGTAGAAAAGATCAAAGGATCT |
| | | TCTTGAGATCCTTTTTTTCTGCGCGTAATCTGCTGCTTGCAAACAAAAA |
| | | AACCACCGCTACCAGCGGTGGTTTGTTTGCCGGATCAAGAGCTACCAAC |
| | | TCTTTTTCCGAAGGTAACTGGCTTCAGCAGAGCGCAGATACCAAATACT |
| | | GTTCTTCTAGTGTAGCCGTAGTTAGGCCACCACTTCAAGAACTCTGTAG |
| | | CACCGCCTACATACCTCGCTCTGCTAATCCTGTTACCAGTGGCTGCTGC |
| | | CAGTGGCGATAAGTCGTGTCTTACCGGGTTGGACTCAAGACGATAGTTA |
| | | CCGGATAAGGCGCAGCGGTCGGGCTGAACGGGGGGTTCGTGCACACAG |
| | | CCCAGCTTGGAGCGAACGACCTACACCGAACTGAGATACCTACAGCGT |
| | | GAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGC |
| | | TTCCAGGGGGAAACGCCTGGTATCTTTATAGTCCTGTCGGGTTTCGCCA |
| | | CCTCTGACTTGAGCGTCGATTTTTGTGATGCTCGTCAGGGGGGCGGAGC |
| | | CTATGGAAAAACGCCAGCAACGCGGCCTTTTTACGGTTCCTGGCCTTTT |
| | | GCTGGCCTTTTGCTCACATGTTCTTTCCTGCGTTATCCCCTGATTCTGTG |
| | | GATAACCGTATTACCGCCTTTGAGTGAGCTGATACCGCTCGCCGCAGCC |
| | | GAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCA |
| | | GC |
| 100 | pFS2- | TGGCACGACAGGTTTCCCGACTGGAAAGCGGGCAGTGAGCGCAACGCA |
| | ggGAPDH_1k | ATTAATGTGAGTTAGCTCACTCATTAGGCACCCCAGGCTTTACACTTTA |
| | b-PTK2- | TGCTTCCGGCTCGTATGTTGTGTGGAATTGTGAGCGGATAACAATTTCA |
| | mmPGK-Neo | CACAGGAAACAGCTATGACCATGATTACGCCAAGCTTAAGCTTGCATG |
| | (Promoters: | CCTGCAGGTCGACCTGCAGCGATCGAACTTGTTTATTGCAGCTTATAAT |
| | rodentia PGK | GGTTACAAATAAAGCAATAGCATCACAAATTTCACAAATAAAGCATTT |
| | (mPGK) and | TTTTCACTGCATTCTAGTTGTGGTTTGTCCAAACTCATCAATGTATCTTA |
| | gallus gallus | TCATGTCTGGATCCCTTCTGAGGGCAGAGGAAGTCTTAATTCAGAAGAA |
| | GAPDH-1kb | CTCGTCAAGAAGGCGATAGAAGGCGATGCGCTGCGAATCGGGAGCGGC |
| | (ggGAPDH_1k)) | GATACCGTAAAGCACGAGGAAGCGGTCAGCCCATTCGCCGCCAAGCTC |
| | | TTCAGCAATATCACGGGTAGCCAACGCTATGTCCTGATAGCGGTCCGCC |
| | | ACACCCAGCCGGCCACAGTCGATGAATCCAGAAAAGCGGCCATTTTCC |
| | | ACCATGATATTCGGCAAGCAGGCATCGCCATGGGTCACGACGAGATCC |
| | | TCGCCGTCGGGCATGCTCGCCTTGAGCCTGGCGAACAGTTCGGCTGGCG |
| | | CGAGCCCCTGATGCTCTTCGTCCAGATCATCCTGATCGACAAGACCGGC |
| | | TTCCATCCGAGTACGTGCTCGCTCGATGCGATGTTTCGCTTGGTGGTCG |
| | | AATGGGCAGGTAGCCGGATCAAGCGTATGCAGCCGCCGCATTGCATCA |
| | | GCCATGATGGATACTTTCTCGGCAGGAGCAAGGTGAGATGACAGGAGA |
| | | TCCTGCCCCGGCACTTCGCCCAATAGCAGCCAGTCCCTTCCCGCTTCAG |
| | | TGACAACGTCGAGCACAGCTGCGCAAGGAACGCCCGTCGTGGCCAGCC |
| | | ACGATAGCCGCGCTGCCTCGTCTTGCAGTTCATTCAGGGCACCGGACAG |
| | | GTCGGTCTTGACAAAAAGAACCGGGCGCCCCTGCGCTGACAGCCGGAA |
| | | CACGGCGGCATCAGAGCAGCCGATTGTCTGTTGTGCCCAGTCATAGCCG |
| | | AATAGCCTCTCCACCCAAGCGGCCGGAGAACCTGCGTGCAATCCATCTT |
| | | GTTCAATCATGCCCATGGTGGCGGCACGTGAGGTAGGTCGAAAGGCCC |
| | | GGAGATGAGGAAGAGGAGAACAGCGCGGCAGACGTGCGCTTTTGAAG |
| | | CGTGCAGAATGCCGGGCCTCCGGAGGACCTTCGGGCGCCCGCCCCGCC |
| | | CCTGAGCCCGCCCCTGAGCCCGCCCCCGGACCCACCCTTCCCAGCTGCT |
| | | GAGCCCAGAAAGCGAAGGAGCAAAGCTGCTATTGGCCGCTGCCCCAAA |
| | | GGCCTACCCGCTTCCATTGCTCAGCGGTGCTGTCCATCTGCACGAGACT |
| | | AGTGAGACGTGCTACTTCCATTTGTCACGTCCTGCACGACGCGAGCTGC |
| | | GGGGCGGGGGGGAACTTCCTGACTAGGGGAGGAGTAGAAGGTGGCGC |
| | | GAAGGGGCCACCAAAGAACGGAGCCGGTTGGCGCTACCGGTGGATGTG |
| | | GAATGTGTGCGAGGCCAGAGGCCACTTGTGTAGCGCCAAGTGCCAGCG |
| | | GGGCTGCTAAAGCGCATGCTCCAGACTGCCTTGGGAAAAGCGCCTCCC |
| | | CTACCCGGTAGAATTGGTGGCGATGCCGAGCTCGGTACCAAGCTTAAG |
| | | TTTAAACGCTAGCAGTAGAATTCGGATCCTACCGTTCGTATAGCATACA |
| | | TTATACGAAGTTATGTCGACGATGTAGGTCACGGTCTCGAAGCCGCGGT |
| | | GCGGGTGCCAGGGCGTGCCCTTGGGCTCCCCGGGCGCGTACTCCACCTC |
| | | ACCCATCTGGTCCATCATGATGAACGGTCGAGGTGGCGGTAGTTGATCC |
| | | CGGCGAACGCGCGGCGCACCGGGAAGCCCTCGCCCTCGAAACCGCTGG |
| | | GCGCGGTGGTCACGGTGAGCACGGGACGTGCGACGGCGTCGGCGGGTG |
| | | CGGATACGCGGGGCAGCGTCAGCGGGTTCTCGACGGTCACGGCGGGCA |
| | | TGTCGACGGCGCGCCGATGATGCTGCAGTTCCTCATTAGCACGGTCCTC |
| | | ACGCAGCACCGCACGGCCTGCTGGGCCGGGCCCGGCCGGGAGCCTCAG |
| | | AGCGGGGCCGTTGGGCGGCCGTGCACCCCGGTATCGCGGTGCGGGGAC |
| | | GGATATGGGACCCAGCGCTCCCCAGCAGCTCGGGGCTGCGCGGCCAAC |
| | | ACGCGCAAGGGCTGAGGACGGGGAGCGGGCAGGCTGCAGCCTCGCATC |
| | | GCGCTCTTTGTCCCGCCCGCGGCCAGACGCCGTTCTCTGGGTACCCGCA |
| | | GGAGCGCAATCCGCACCCACTGTACCGCATCGCGTGATGCAGCCGCAT |
| | | CGCCCCGGCCTCGTCCGAGGACGCCACGGCCCCACGTGGGGCTTTGTCT |
| | | GCACGCAGCTCCGCGCCCCCCGGCCCCGGGGCACCCCTCGCCGCCCCC |
| | | ACCCCGCACGGGCACCGTGGTACGGCAGCAACCCCCTAAGCCCACCCT |
| | | GAGCCCAACGGCCGCCCTTTGTTGCGAGGGGGGAGGGGGAAGGAGAG |
| | | GAGAGGGGGGGCCGTTTCGCCCGCACCGTGCGCGCACGTAGCCCCGAG |
| | | CCCGCCCCCGCGCTGCCATTCGGCGCTGGCCCGGCGGGGCGGGGCGCG |
| | | GGGCGCTGCCTCCGTGTCTATAAAGGGCGGCGGCGCGGCACTGCGCCA |
| | | CCTTCTCACTGCGCGCTGGGGCCGTTGACGTGCAGCAGGTAACGGGCG |
| | | GGGGGACGTGGGGGCGGAGAAGCGTACCCCCCACCCACCCGCCTGTCT |
| | | GCCTGCGCCCCCCTGTAGGGGTTGGGGCTGTAGGACCGCGAGGAGTGC |
| | | GGCGGGGGAAGGGGGGAGGGGGTCTCCGCGCAGGACCGCGTGGTGCC |
| | | CGGGGCACAAAGGCGGGGGGGGGGGGCGCGAGATGAAGGGGGTGGGG |
| | | GTTGGGGGAGAGAGGGAGAACTGCGGGGGAGAGAGCTCGATGGGGAT |
| | | GAAGGAAGGGATGGCGGGTCGGGGGGCGGTGCCCAGAGCTCCGCAGC |
| | | GCGCGCGGTGGGGGGACGTTACTGGGCCGGCGGTCTGCGTGCGGCACC |
| | | GCACGTCTCACTGCCACGTAGTCGACTCGAGGCAGGAAAATGGCAGCA |
| | | GCTTACCTTGATCCAAACTTGAATCATACACCAAGTTCAAGTGCAAAGA |
| | | CGCACCTCGGTACTGGGATGGAGCGTTCCCCGGGGGCCATGGAGCGAG |
| | | TCCTAAAGGTTTTTCACTACTTTGAAAACAGCAGCGAGCCAACGACGTG |
| | | GGCCAGCATTATCCGGCATGGAGATGCTACTGATGTTCGAGGCATAAT |
| | | ACAGAAGATTGTGGACTGTCACAAAGTGAAAAATGTGGCCTGCTATGG |
| | | GTTGCGACTCAGTCATCTGCAGTCTGAGGAGGTTCACTGGCTGCACCTG |
| | | GACATGGGGGTATCCAATGTGAGAGAGAAATTTGAACTAGCACATCCT |
| | | CCAGAAGAATGGAAATATGAACTGAGAATTCGGTACCTGCCCAAAGGA |
| | | TTTCTAAACCAGTTCACTGAGGACAAACCAACTTTAAATTTTTTCTATC |
| | | AGCAGGTGAAAAATGACTATATGTTAGAAATAGCAGATCAAGTGGACC |
| | | AGGAAATTGCTTTGAAACTAGGTTGCCTTGAAATCAGGAGATCCTACG |
| | | GAGAGATGAGAGGCAATGCATTAGAGAAGAAATCCAACTATGAAGTGC |
| | | TAGAAAAAGATGTCGGTTTAAGACGATTTTTTCCGAAGAGTTTGCTAGA |
| | | TTCAGTGAAGGCCAAAACACTACGAAAATTAATCCAACAGACATTTCG |
| | | ACAATTTGCCAACCTCAACAGAGAAGAAAGTATTTTGAAATTCTTTGAG |
| | | ATCCTCTCTCCAGTGTACAGATTTGACAAGGAATGCTTCAAGTGTGCCC |
| | | TTGGTTCAAGCTGGATTATTTCAGTGGAGCTGGCAATTGGCCCAGAGGA |
| | | AGGAATCAGCTACCTTACAGACAAGGGTGCAAATCCAACTCACCTGGC |
| | | AGATTTTAATCAAGTACAAACTATTCAGTATTCAAACAGTGAAGACAA |
| | | GGACAGAAAAGGGATGTTGCAACTGAAGATAGCTGGTGCACCTGAGCC |
| | | TCTGACAGTGACAGCACCATCCTTAACCATTGCAGAGAATATGGCTGAC |
| | | TTGATAGACGGATACTGCCGACTGGTGAATGGAGCCACGCAATCTTTTA |
| | | TTATCAGGCCACAGAAAGAAGGTGAAAGAGCTTTACCATCAATACCAA |
| | | AGCTGGCCAACAATGAGAAGCAAGGAGTAAGGTCGCACACAGTCTCTG |
| | | TATCAGAAACAGATGACTATGCAGAGATAATAGATGAAGAAGATACTT |
| | | ATACAATGCCATCAACCAGAGATTATGAAATTCAAAGGGAGAGAATTG |
| | | AACTGGGGCGCTGCATTGGTGAAGGACAGTTTGGAGATGTGCACCAAG |
| | | GAATTTACATGAGTCCGGAAAATCCAGCTATGGCTGTAGCAATCAAAA |
| | | CATGTAAAAACTGCACCTCAGACAGCGTTAGAGAAAAGTTCCTACAAG |
| | | AAGCCTTAACAATGCGTCAGTTTGATCATCCTCACATTGTGAAGCTCAT |
| | | TGGAGTTATTACAGAAAACCCAGTGTGGATAATCATGGAGCTCTGTAC |
| | | ACTTGGAGAGTTGAGATCGTTTCTGCAAGTAAGAAAATTCAGCTTGGAC |
| | | CTGGCCTCCCTCATCCTCTACGCTTACCAGCTTAGCACAGCACTTGCTTA |
| | | CCTAGAGAGCAAAAGATTTGTACATAGAGATATTGCTGCTAGGAACGT |
| | | GCTGGTATCTGCCACTGACTGTGTGAAATTGGGTGACTTTGGCTTATCC |
| | | CGATACATGGAAGACAGTACTTACTATAAAGCTTCCAAAGGAAAGTTA |
| | | CCTATCAAATGGATGGCTCCAGAGTCAATCAACTTCCGACGGTTTACCT |
| | | CAGCAAGCGATGTGTGGATGTTTGGTGTGTGTATGTGGGAGATCCTGAT |
| | | GCATGGGGTAAAGCCCTTCCAGGGAGTGAAAAATAATGATGTTATTGG |
| | | TCGGATTGAGAACGGTGAGCGGCTCCCCATGCCTCCGAACTGCCCTCCC |
| | | ACCCTCTACAGCCTTATGACCAAGTGCTGGGCATACGACCCTAGTAGAC |
| | | GACCCAGGTTTACTGAACTTAAAGCACAACTCAGTACAATACTGGAGG |
| | | AGGAGAAGCTGCAGCAAGAGGAACGAATGAGAATGGAATCCAGGCGA |
| | | CAAGTCACAGTATCCTGGGACTCAGGAGGATCAGATGAAGCTCCTCCC |
| | | AAGCCCAGCAGGCCTGGTTACCCCAGCCCAAGGTCCAGTGAAGGGTTT |
| | | TATCCGAGTCCTCAGCATATGGTACAGCCAAATCACTACCAGGTATCTG |
| | | GCTACTCTGGTTCTCATGGGATACCAGCCATGGCAGGCAGCATTTATCC |
| | | TGGGCAAGCTTCTCTCTTGGATCAAACAGATTCCTGGAACCATCGACCT |
| | | CAGGAAGTATCAGCATGGCAGCCAAACATGGAGGATTCGGGCACTTTG |
| | | GATGTACGAGGAATGGGGCAGGTTCTGCCCACACATCTCATGGAGGAG |
| | | AGGTTAATAAGACAACAGCAAGAGATGGAAGAAGATCAACGCTGGCTT |
| | | GAGAAAGAGGAACGATTCCTGGTAATGAAACCTGATGTGCGGCTCTCC |
| | | AGAGGCAGCATTGAACGGGAGGACGGAGGTCTCCAGGGCCCAGCTGGT |
| | | AACCAGCACATATATCAGCCTGTGGGTAAACCAGATCATGCCGCTCCA |
| | | CCAAAGAAGCCCCCTCGCCCTGGAGCCCCTCACTTGGGCAGCCTCGCG |
| | | AGCCTGAACAGCCCCGTGGACAGCTACAACGAAGGCGTGAAGATCAAG |
| | | CCACAGGAAATCAGCCCTCCTCCTACGGCCAACCTGGACCGCTCCAATG |
| | | ACAAAGTCTATGAGAATGTAACCGGGCTGGTGAAAGCTGTCATAGAGA |
| | | TGTCCAGTAAAATACAGCCAGCTCCGCCAGAGGAGTACGTGCCCATGG |
| | | TAAAGGAGGTTGGCTTGGCGCTGAGAACCTTGCTAGCAACAGTGGATG |
| | | AGTCGCTGCCAGTGCTTCCTGCAAGCACCCACAGAGAGATTGAGATGG |
| | | CCCAGAAACTGCTGAACTCTGACCTGGCTGAGCTCATTAACAAGATGA |
| | | AGCTGGCCCAGCAGTACGTCATGACCAGCCTGCAGCAGGAGTACAAGA |
| | | AGCAAATGCTGACGGCTGCTCATGCTCTGGCTGTGGATGCCAAGAACTT |
| | | GCTGGATGTCATCGATCAAGCCAGACTGAAAATGATCAGCCAGTCCAG |
| | | GCCCCACTAATAAGATCGTATCGTTAACGCGGCCGCCACGTGCTCGAG |
| | | GAGTTGCCAGCCATCTGTTGTTTGCCCCTCCCCCGTGCCTTCCTTGACCC |
| | | TGGAAGGTGCCACTCCCACTGTCCTTTCCTAATAAAATGAGGAAATTGC |
| | | ATCATAACTTCGTATAGCATACATTATACGAACGGTAGGATCCCCGGGT |
| | | ACCGAGCTCGAATTCACTGGCCGTCGTTTTACAACGTCGTGACTGGGAA |
| | | AACCCTGGCGTTACCCAACTTAATCGCCTTGCAGCACATCCCCCTTTCG |
| | | CCAGCTGGCGTAATAGCGAAGAGGCCCGCACCGATCGCCCTTCCCAAC |
| | | AGTTGCGCAGCCTGAATGGCGAATGGCGCCTGATGCGGTATTTTCTCCT |
| | | TACGCATCTGTGCGGTATTTCACACCGCATATGGTGCACTCTCAGTACA |
| | | ATCTGCTCTGATGCCGCATAGTTAAGCCAGCCCCGACACCCGCCAACAC |
| | | CCGCTGACGCGCCCTGACGGGCTTGTCTGCTCCCGGCATCCGCTTACAG |
| | | ACAAGCTGTGACCGTCTCCGGGAGCTGCATGTGTCAGAGGTTTTCACCG |
| | | TCATCACCGAAACGCGCGAGACGAAAGGGCCTCGTGATACGCCTATTT |
| | | TTATAGGTTAATGTCATGATAATAATGGTTTCTTAGACGTCAGGTGGCA |
| | | CTTTTCGGGGAAATGTGCGCGGAACCCCTATTTGTTTATTTTTCTAAATA |
| | | CATTCAAATATGTATCCGCTCATGAGACAATAACCCTGATAAATGCTTC |
| | | AATAATATTGAAAAAGGAAGAGTATGAGTATTCAACATTTCCGTGTCG |
| | | CCCTTATTCCCTTTTTTGCGGCATTTTGCCTTCCTGTTTTTGCTCACCCAG |
| | | AAACGCTGGTGAAAGTAAAAGATGCTGAAGATCAGTTGGGTGCACGAG |
| | | TGGGTTACATCGAACTGGATCTCAACAGCGGTAAGATCCTTGAGAGTTT |
| | | TCGCCCCGAAGAACGTTTTCCAATGATGAGCACTTTTAAAGTTCTGCTA |
| | | TGTGGCGCGGTATTATCCCGTATTGACGCCGGGCAAGAGCAACTCGGTC |
| | | GCCGCATACACTATTCTCAGAATGACTTGGTTGAGTACTCACCAGTCAC |
| | | AGAAAAGCATCTTACGGATGGCATGACAGTAAGAGAATTATGCAGTGC |
| | | TGCCATAACCATGAGTGATAACACTGCGGCCAACTTACTTCTGACAACG |
| | | ATCGGAGGACCGAAGGAGCTAACCGCTTTTTTGCACAACATGGGGGAT |
| | | CATGTAACTCGCCTTGATCGTTGGGAACCGGAGCTGAATGAAGCCATA |
| | | CCAAACGACGAGCGTGACACCACGATGCCTGTAGCAATGGCAACAACG |
| | | TTGCGCAAACTATTAACTGGCGAACTACTTACTCTAGCTTCCCGGCAAC |
| | | AATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGG |
| | | TGAGCGTGGCTCTCGCGGTATCATTGCAGCACTGGGGCCAGATGGTAA |
| | | GCCCTCCCGTATCGTAGTTATCTACACGACGGGGAGTCAGGCAACTATG |
| | | GATGAACGAAATAGACAGATCGCTGAGATAGGTGCCTCACTGATTAAG |
| | | CATTGGTAACTGTCAGACCAAGTTTACTCATATATACTTTAGATTGATTT |
| | | AAAACTTCATTTTTAATTTAAAAGGATCTAGGTGAAGATCCTTTTTGAT |
| | | AATCTCATGACCAAAATCCCTTAACGTGAGTTTTCGTTCCACTGAGCGT |
| | | CAGACCCCGTAGAAAAGATCAAAGGATCTTCTTGAGATCCTTTTTTTCT |
| | | GCGCGTAATCTGCTGCTTGCAAACAAAAAAACCACCGCTACCAGCGGT |
| | | GGTTTGTTTGCCGGATCAAGAGCTACCAACTCTTTTTCCGAAGGTAACT |
| | | GGCTTCAGCAGAGCGCAGATACCAAATACTGTTCTTCTAGTGTAGCCGT |
| | | AGTTAGGCCACCACTTCAAGAACTCTGTAGCACCGCCTACATACCTCGC |
| | | TCTGCTAATCCTGTTACCAGTGGCTGCTGCCAGTGGCGATAAGTCGTGT |
| | | CTTACCGGGTTGGACTCAAGACGATAGTTACCGGATAAGGCGCAGCGG |
| | | TCGGGCTGAACGGGGGGTTCGTGCACACAGCCCAGCTTGGAGCGAACG |
| | | ACCTACACCGAACTGAGATACCTACAGCGTGAGCTATGAGAAAGCGCC |
| | | ACGCTTCCCGAAGGGAGAAAGGCGGACAGGTATCCGGTAAGCGGCAG |
| | | GGTCGGAACAGGAGAGCGCACGAGGGAGCTTCCAGGGGGAAACGCCT |
| | | GGTATCTTTATAGTCCTGTCGGGTTTCGCCACCTCTGACTTGAGCGTCG |
| | | ATTTTTGTGATGCTCGTCAGGGGGGCGGAGCCTATGGAAAAACGCCAG |
| | | CAACGCGGCCTTTTTACGGTTCCTGGCCTTTTGCTGGCCTTTTGCTCACA |
| | | TGTTCTTTCCTGCGTTATCCCCTGATTCTGTGGATAACCGTATTACCGCC |
| | | TTTGAGTGAGCTGATACCGCTCGCCGCAGCCGAACGACCGAGCGCAGC |
| | | GAGTCAGTGAGCGAGGAAGCGGAAGAGCGCCCAATACGCAAACCGCC |
| | | TCTCCCCGCGCGTTGGCCGATTCATTAATGCAGC |
| 101 | pFS2- | TGGCACGACAGGTTTCCCGACTGGAAAGCGGGCAGTGAGCGCAACGCA |
| | ggGAPDH_1k | ATTAATGTGAGTTAGCTCACTCATTAGGCACCCCAGGCTTTACACTTTA |
| | b-SPHK1- | TGCTTCCGGCTCGTATGTTGTGTGGAATTGTGAGCGGATAACAATTTCA |
| | mmPGK-Neo | CACAGGAAACAGCTATGACCATGATTACGCCAAGCTTAAGCTTGCATG |
| | (Promoters: | CCTGCAGGTCGACCTGCAGCGATCGAACTTGTTTATTGCAGCTTATAAT |
| | rodentia PGK | GGTTACAAATAAAGCAATAGCATCACAAATTTCACAAATAAAGCATTT |
| | (mPGK) and | TTTTCACTGCATTCTAGTTGTGGTTTGTCCAAACTCATCAATGTATCTTA |
| | gallus gallus | TCATGTCTGGATCCCTTCTGAGGGCAGAGGAAGTCTTAATTCAGAAGAA |
| | GAPDH-1kb | CTCGTCAAGAAGGCGATAGAAGGCGATGCGCTGCGAATCGGGAGCGGC |
| | (ggGAPDH_1k)) | GATACCGTAAAGCACGAGGAAGCGGTCAGCCCATTCGCCGCCAAGCTC |
| | | TTCAGCAATATCACGGGTAGCCAACGCTATGTCCTGATAGCGGTCCGCC |
| | | ACACCCAGCCGGCCACAGTCGATGAATCCAGAAAAGCGGCCATTTTCC |
| | | ACCATGATATTCGGCAAGCAGGCATCGCCATGGGTCACGACGAGATCC |
| | | TCGCCGTCGGGCATGCTCGCCTTGAGCCTGGCGAACAGTTCGGCTGGCG |
| | | CGAGCCCCTGATGCTCTTCGTCCAGATCATCCTGATCGACAAGACCGGC |
| | | TTCCATCCGAGTACGTGCTCGCTCGATGCGATGTTTCGCTTGGTGGTCG |
| | | AATGGGCAGGTAGCCGGATCAAGCGTATGCAGCCGCCGCATTGCATCA |
| | | GCCATGATGGATACTTTCTCGGCAGGAGCAAGGTGAGATGACAGGAGA |
| | | TCCTGCCCCGGCACTTCGCCCAATAGCAGCCAGTCCCTTCCCGCTTCAG |
| | | TGACAACGTCGAGCACAGCTGCGCAAGGAACGCCCGTCGTGGCCAGCC |
| | | ACGATAGCCGCGCTGCCTCGTCTTGCAGTTCATTCAGGGCACCGGACAG |
| | | GTCGGTCTTGACAAAAAGAACCGGGCGCCCCTGCGCTGACAGCCGGAA |
| | | CACGGCGGCATCAGAGCAGCCGATTGTCTGTTGTGCCCAGTCATAGCCG |
| | | AATAGCCTCTCCACCCAAGCGGCCGGAGAACCTGCGTGCAATCCATCTT |
| | | GTTCAATCATGCCCATGGTGGCGGCACGTGAGGTAGGTCGAAAGGCCC |
| | | GGAGATGAGGAAGAGGAGAACAGCGCGGCAGACGTGCGCTTTTGAAG |
| | | CGTGCAGAATGCCGGGCCTCCGGAGGACCTTCGGGCGCCCGCCCCGCC |
| | | CCTGAGCCCGCCCCTGAGCCCGCCCCCGGACCCACCCTTCCCAGCTGCT |
| | | GAGCCCAGAAAGCGAAGGAGCAAAGCTGCTATTGGCCGCTGCCCCAAA |
| | | GGCCTACCCGCTTCCATTGCTCAGCGGTGCTGTCCATCTGCACGAGACT |
| | | AGTGAGACGTGCTACTTCCATTTGTCACGTCCTGCACGACGCGAGCTGC |
| | | GGGGCGGGGGGGAACTTCCTGACTAGGGGAGGAGTAGAAGGTGGCGC |
| | | GAAGGGGCCACCAAAGAACGGAGCCGGTTGGCGCTACCGGTGGATGTG |
| | | GAATGTGTGCGAGGCCAGAGGCCACTTGTGTAGCGCCAAGTGCCAGCG |
| | | GGGCTGCTAAAGCGCATGCTCCAGACTGCCTTGGGAAAAGCGCCTCCC |
| | | CTACCCGGTAGAATTGGTGGCGATGCCGAGCTCGGTACCAAGCTTAAG |
| | | TTTAAACGCTAGCAGTAGAATTCGGATCCTACCGTTCGTATAGCATACA |
| | | TTATACGAAGTTATGTCGACGATGTAGGTCACGGTCTCGAAGCCGCGGT |
| | | GCGGGTGCCAGGGCGTGCCCTTGGGCTCCCCGGGCGCGTACTCCACCTC |
| | | ACCCATCTGGTCCATCATGATGAACGGTCGAGGTGGCGGTAGTTGATCC |
| | | CGGCGAACGCGCGGCGCACCGGGAAGCCCTCGCCCTCGAAACCGCTGG |
| | | GCGCGGTGGTCACGGTGAGCACGGGACGTGCGACGGCGTCGGCGGGTG |
| | | CGGATACGCGGGGCAGCGTCAGCGGGTTCTCGACGGTCACGGCGGGCA |
| | | TGTCGACGGCGCGCCGATGATGCTGCAGTTCCTCATTAGCACGGTCCTC |
| | | ACGCAGCACCGCACGGCCTGCTGGGCCGGGCCCGGCCGGGAGCCTCAG |
| | | AGCGGGGCCGTTGGGCGGCCGTGCACCCCGGTATCGCGGTGCGGGGAC |
| | | GGATATGGGACCCAGCGCTCCCCAGCAGCTCGGGGCTGCGCGGCCAAC |
| | | ACGCGCAAGGGCTGAGGACGGGGAGCGGGCAGGCTGCAGCCTCGCATC |
| | | GCGCTCTTTGTCCCGCCCGCGGCCAGACGCCGTTCTCTGGGTACCCGCA |
| | | GGAGCGCAATCCGCACCCACTGTACCGCATCGCGTGATGCAGCCGCAT |
| | | CGCCCCGGCCTCGTCCGAGGACGCCACGGCCCCACGTGGGGCTTTGTCT |
| | | GCACGCAGCTCCGCGCCCCCCGGCCCCGGGGCACCCCTCGCCGCCCCC |
| | | ACCCCGCACGGGCACCGTGGTACGGCAGCAACCCCCTAAGCCCACCCT |
| | | GAGCCCAACGGCCGCCCTTTGTTGCGAGGGGGGAGGGGGAAGGAGAG |
| | | GAGAGGGGGGGCCGTTTCGCCCGCACCGTGCGCGCACGTAGCCCCGAG |
| | | CCCGCCCCCGCGCTGCCATTCGGCGCTGGCCCGGCGGGGCGGGGCGCG |
| | | GGGCGCTGCCTCCGTGTCTATAAAGGGCGGCGGCGCGGCACTGCGCCA |
| | | CCTTCTCACTGCGCGCTGGGGCCGTTGACGTGCAGCAGGTAACGGGCG |
| | | GGGGGACGTGGGGGCGGAGAAGCGTACCCCCCACCCACCCGCCTGTCT |
| | | GCCTGCGCCCCCCTGTAGGGGTTGGGGCTGTAGGACCGCGAGGAGTGC |
| | | GGCGGGGGAAGGGGGGAGGGGGTCTCCGCGCAGGACCGCGTGGTGCC |
| | | CGGGGCACAAAGGCGGGGGCGGGCGGCGCGAGATGAAGGGGGTGGGG |
| | | GTTGGGGGAGAGAGGGAGAACTGCGGGGGAGAGAGCTCGATGGGGAT |
| | | GAAGGAAGGGATGGCGGGTCGGGGGGCGGTGCCCAGAGCTCCGCAGC |
| | | GCGCGCGGTGGGGGGACGTTACTGGGCCGGCGGTCTGCGTGCGGCACC |
| | | GCACGTCTCACTGCCACGTAGTCGACTCGAGGCAGGAAAATGGCCGCC |
| | | GGCCGCCGGGTCCCCCCGCACGGCGACGGCGGTGCGGTGCTGCTGCGG |
| | | GGCCGCTTCTCCCCGGGCCCGTCTCCCGGTCCGGCCGCGTGGTCCGTCG |
| | | CTCTGACCGAACGGGAGCTGCAGGTGCTGCGTCCCGGCGGTCCCGACG |
| | | CGGCGCTCCGTTTGGCCGATTGCGTGGGCAGCGCCGCGTTCCCGTCCGG |
| | | CCCCGCCGCCGCCTGCTTCTCCCTCGTGGGTTACCCGTTCCGTAAGGCC |
| | | CGATGGGGTTCCCCCGCCCGGCAACGCCTGCAGAGGACTTTCCGCGTTT |
| | | CCCAACACCCCAACGCCGAGGAGAATCTCCGCATCGCCCAGGCCTGGA |
| | | GCCAACGCATCCGAGAGCTCTCGCTGCCCGCAGCGCCCACGCGGGACG |
| | | GTGTCAGCTATGGGCTGCTGCCCCGGCCCTGCCGAGCTCTGGTGCTGCT |
| | | GAACCCGCAGAGCGGCGCTGGCCGCGCCCTTGAGGACTTCCAGGCGGT |
| | | GGTGCAGCCCATGCTGGCTGATGCAGACATCGCAGCCACCGTCTTTGTC |
| | | ACCGAGAGACCCCACCACGCTCAGGAGAAGGTGCGGGATGAAGACCTG |
| | | TCGCAGTGGGATGTGCTGGTGGTGATGTCTGGGGATGGTCTGCTCCACG |
| | | AGGTGCTGAATGGGCTGATGGAGCGGCCGGACTGGGAGGAGGCCCTGC |
| | | AGACACCGCTGTGCATCCTGCCCGGGGGCTCCGGGAATGCCCTGGCTG |
| | | CCTCCATCAACTACTATGCGGGCAACGACCACGTGGCCAAGAAGAAAC |
| | | TGCTGACCAACTGCACCTTCATCCTGTGCAAAGGGCTGCACGCCCCGAT |
| | | GGACCTGGTCTCGTTGAGCACAGCCTCGGGCAAACGCTTCTTCTCCTTC |
| | | CTGGGCTTCGGATGGGGTTTCATCGCCGACGTGGACATCGACAGCGAG |
| | | AAGTACCGGCGGCTGGGCAACGCGCGCTTCACGCTGGGCACGCTGCAG |
| | | TGCTTGGTGCGCCTGCGCGTCTACCAGGGCCGCCTCTCCTACCTGCCTG |
| | | CGGCCACCGAGCCGGGCACCCCCAGGGACCGCAAGGCGCCCGCCCGGC |
| | | GGGCCGCACCAACAGCGGGGACGGAGGCGCCGAGCGGGCACGGGGCG |
| | | CTGCCCGCTGACACGCTGCTGGTGCCGCTGGGGCAGCCGGTGCCGCCG |
| | | CATTGGGTGGTGGTGCCCGAGGAGGAGTTCGTCTGCGTCTACGCCATCT |
| | | ACCAGTCCCACCTGGGCACCAACCTGCTGATGGCCCCGGCGGCGCGGC |
| | | TGCACGACGGCTGCATCCACCTCTTCTACGTGGGGGCGGGCATCAGCCG |
| | | CGTGGCGCTGCTGAAGATCTTCCTGGCCATGGGCAGGGGCACGCACCT |
| | | GGATCTGAACTGCCCCCACCTGCACTGCGTCCCGGTGCGCGCCTTCCGC |
| | | CTGGAGCCGCGGGGCAGCGCCGGCATCATGACAGTGGACGGAGAGGC |
| | | GTTGGCGTGCGAGCCCGTGCAGGGGCAGATCCACGGCCACATGTGCCG |
| | | CGTGGTCACCGCTTCGTAGTAAGATCGTATCGTTAACGCGGCCGCCACG |
| | | TGCTCGAGGAGTTGCCAGCCATCTGTTGTTTGCCCCTCCCCCGTGCCTTC |
| | | CTTGACCCTGGAAGGTGCCACTCCCACTGTCCTTTCCTAATAAAATGAG |
| | | GAAATTGCATCATAACTTCGTATAGCATACATTATACGAACGGTAGGAT |
| | | CCCCGGGTACCGAGCTCGAATTCACTGGCCGTCGTTTTACAACGTCGTG |
| | | ACTGGGAAAACCCTGGCGTTACCCAACTTAATCGCCTTGCAGCACATCC |
| | | CCCTTTCGCCAGCTGGCGTAATAGCGAAGAGGCCCGCACCGATCGCCCT |
| | | TCCCAACAGTTGCGCAGCCTGAATGGCGAATGGCGCCTGATGCGGTATT |
| | | TTCTCCTTACGCATCTGTGCGGTATTTCACACCGCATATGGTGCACTCTC |
| | | AGTACAATCTGCTCTGATGCCGCATAGTTAAGCCAGCCCCGACACCCGC |
| | | CAACACCCGCTGACGCGCCCTGACGGGCTTGTCTGCTCCCGGCATCCGC |
| | | TTACAGACAAGCTGTGACCGTCTCCGGGAGCTGCATGTGTCAGAGGTTT |
| | | TCACCGTCATCACCGAAACGCGCGAGACGAAAGGGCCTCGTGATACGC |
| | | CTATTTTTATAGGTTAATGTCATGATAATAATGGTTTCTTAGACGTCAG |
| | | GTGGCACTTTTCGGGGAAATGTGCGCGGAACCCCTATTTGTTTATTTTTC |
| | | TAAATACATTCAAATATGTATCCGCTCATGAGACAATAACCCTGATAAA |
| | | TGCTTCAATAATATTGAAAAAGGAAGAGTATGAGTATTCAACATTTCCG |
| | | TGTCGCCCTTATTCCCTTTTTTGCGGCATTTTGCCTTCCTGTTTTTGCTCA |
| | | CCCAGAAACGCTGGTGAAAGTAAAAGATGCTGAAGATCAGTTGGGTGC |
| | | ACGAGTGGGTTACATCGAACTGGATCTCAACAGCGGTAAGATCCTTGA |
| | | GAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAAC |
| | | TCGGTCGCCGCATACACTATTCTCAGAATGACTTGGTTGAGTACTCACC |
| | | AGTCACAGAAAAGCATCTTACGGATGGCATGACAGTAAGAGAATTATG |
| | | CAGTGCTGCCATAACCATGAGTGATAACACTGCGGCCAACTTACTTCTG |
| | | ACAACGATCGGAGGACCGAAGGAGCTAACCGCTTTTTTGCACAACATG |
| | | GGGGATCATGTAACTCGCCTTGATCGTTGGGAACCGGAGCTGAATGAA |
| | | GCCATACCAAACGACGAGCGTGACACCACGATGCCTGTAGCAATGGCA |
| | | ACAACGTTGCGCAAACTATTAACTGGCGAACTACTTACTCTAGCTTCCC |
| | | GGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGG |
| | | AGCCGGTGAGCGTGGCTCTCGCGGTATCATTGCAGCACTGGGGCCAGA |
| | | TGGTAAGCCCTCCCGTATCGTAGTTATCTACACGACGGGGAGTCAGGCA |
| | | ACTATGGATGAACGAAATAGACAGATCGCTGAGATAGGTGCCTCACTG |
| | | ATTAAGCATTGGTAACTGTCAGACCAAGTTTACTCATATATACTTTAGA |
| | | TTGATTTAAAACTTCATTTTTAATTTAAAAGGATCTAGGTGAAGATCCT |
| | | TTTTGATAATCTCATGACCAAAATCCCTTAACGTGAGTTTTCGTTCCACT |
| | | GAGCGTCAGACCCCGTAGAAAAGATCAAAGGATCTTCTTGAGATCCTTT |
| | | TTTTCTGCGCGTAATCTGCTGCTTGCAAACAAAAAAACCACCGCTACCA |
| | | GCGGTGGTTTGTTTGCCGGATCAAGAGCTACCAACTCTTTTTCCGAAGG |
| | | TAACTGGCTTCAGCAGAGCGCAGATACCAAATACTGTTCTTCTAGTGTA |
| | | GCCGTAGTTAGGCCACCACTTCAAGAACTCTGTAGCACCGCCTACATAC |
| | | CTCGCTCTGCTAATCCTGTTACCAGTGGCTGCTGCCAGTGGCGATAAGT |
| | | CGTGTCTTACCGGGTTGGACTCAAGACGATAGTTACCGGATAAGGCGC |
| | | AGCGGTCGGGCTGAACGGGGGGTTCGTGCACACAGCCCAGCTTGGAGC |
| | | GAACGACCTACACCGAACTGAGATACCTACAGCGTGAGCTATGAGAAA |
| | | GCGCCACGCTTCCCGAAGGGAGAAAGGCGGACAGGTATCCGGTAAGCG |
| | | GCAGGGTCGGAACAGGAGAGCGCACGAGGGAGCTTCCAGGGGGAAAC |
| | | GCCTGGTATCTTTATAGTCCTGTCGGGTTTCGCCACCTCTGACTTGAGCG |
| | | TCGATTTTTGTGATGCTCGTCAGGGGGGCGGAGCCTATGGAAAAACGC |
| | | CAGCAACGCGGCCTTTTTACGGTTCCTGGCCTTTTGCTGGCCTTTTGCTC |
| | | ACATGTTCTTTCCTGCGTTATCCCCTGATTCTGTGGATAACCGTATTACC |
| | | GCCTTTGAGTGAGCTGATACCGCTCGCCGCAGCCGAACGACCGAGCGC |
| | | AGCGAGTCAGTGAGCGAGGAAGCGGAAGAGCGCCCAATACGCAAACC |
| | | GCCTCTCCCCGCGCGTTGGCCGATTCATTAATGCAGC |

1. A method for increasing cell density of a culture comprising a myogenic cell line, comprising:
(a) co-culturing a myogenic cell with a support cell, wherein the support cell line comprises a polynucleotide comprising a coding sequence of a gene of interest; and
(b) culturing the myogenic cell and the support cell in a cultivation infrastructure under conditions sufficient to induce proliferation of the myogenic cell, thereby increasing cell density of the culture.

2. The method of claim 1, wherein the myogenic cell and the support cell are co-cultured at a ratio of 10:1 to 1:10 number of myogenic cells to number of support cells.

3. The method of claim 1, wherein the myogenic cell is selected from: a myoblast, a myocyte, a satellite cell, a side population cell, a myogenic pericyte, a mesangioblast, a multinucleated myotube, a skeletal muscle fiber, or a combination thereof.

4. The method of claim 1, wherein the myogenic cells are natively myogenic or are non-natively myogenic.

5. The method of claim 1, wherein the support cell is selected from: a fibroblast, a myofibroblast, a mesenchymal cell, an epithelial cell, and a stromal cell.

6. The method of claim 1, wherein the gene of interest is selected from: FAP, IGF2, SDC4, SPHK1, and FAK, or a combination thereof.

7. The method of claim 1, wherein the myogenic cell comprises a polynucleotide comprising a coding sequence of a gene of interest.

8. The method of claim 7, wherein the gene of interest is IGF2 or genetic variant thereof.

9. The method of claim 1, wherein a myogenic cell co-cultured with the support cell comprising a polynucleotide comprising a coding sequence of a gene of interest comprises a higher proliferation rate as compared to a myogenic cell not cultured with a support cell comprising a polynucleotide comprising a coding sequence of a gene of interest.

10. The method of claim 1, wherein the myogenic cells, the support cells, or both, are immortalized.

11. The method of claim 1, further comprising an immortalizing step, wherein the myogenic cells, the support cells, or both are immortalized.

12. The method of claim 10, wherein the immortalization is selected from a method comprising: transducing with a polynucleotide encoding TERT, transducing with a polynucleotide encoding CDK4/6, transducing with a polynucleotide Cyclin D1, inactivating a gene encoding an inhibitor of cyclin-dependent kinase 4/6 (CDK4/6), inactivating a gene encoding an inhibitor of Cyclin D1, or a combination thereof.

13. The method of claim 1, wherein the co-culturing, culturing steps, or both, comprises contacting the myogenic cell, support cell, or both with a growth medium.

14. The method of claim 13, wherein the growth media comprises one or more of: DMEM/F12, fetal bovine serum, chicken serum, fibroblast growth factor 2, a TGF-beta inhibitor, an activin A inhibitor, and a WNT activator.

15. The method of claim 1, wherein the co-culturing and/or culturing steps comprises contacting the myogenic cell, support cell, or both with a differentiation medium comprising bovine serum, chicken serum, horse serum, or a combination thereof.

16. The method of claim 1, wherein the myogenic cells are from a chicken, a duck, turkey, porcine, or bovine.

17. The method of claim 1, wherein the support cells are from a chicken, a duck, or turkey, porcine, or bovine.

18. The method of claim 1, further comprising:
inducing myogenic specific differentiation, wherein the differentiated cells form myocytes and multinucleated myotubes, wherein the myocytes and multinucleated myotubes form a skeletal muscle fiber, and
isolating the skeletal muscle fiber and producing a cell based meat product suitable for consumption.

19. A cell-based meat product suitable for consumption produced using the method of claim 1.

20. The cell-based meat product of claim 19, wherein the cell-based meat product suitable for consumption is a raw, uncooked food product or a cooked food product.
